(19) United States
(12) Patent Application Publication
(10) Pub. No.: US 2019/0388425 A1
(43) Pub. Date: Dec. 26, 2019

(54) SUBSTITUTED IMIDAZOPYRIDINPYRIMIDINES

(71) Applicants: Bayer Pharma Aktiengesellschaft, Berlin (DE); Bayer Aktiengesellschaft, Leverkusen (DE)

(72) Inventors: Ulrich KLAR, Berlin (DE); Heike SCHÄCKE, Berlin (DE); Detlev SÜLZLE, Berlin (DE); Stephan MENZ, Berlin (DE)

(73) Assignees: Bayer Pharma Aktiengesellschaft, Berlin (DE); Bayer Aktiengesellschaft, Leverkusen (DE)

(21) Appl. No.: 16/479,400

(22) PCT Filed: Jan. 19, 2018
(86) PCT No.: PCT/EP2018/051266

§ 371 Date: Jul. 19, 2019

(30) Foreign Application Priority Data
Jan. 20, 2017 (EP) 17152508.2
Jun. 30, 2017 (EP) 17179211.2
Aug. 29, 2017 (EP) 17188391.1

(51) Int. Cl. A61K 31/519; A61K 31/5386; C07D 519/00; A61P 35/00; A61P 15/02; A61P 29/00

(52) U.S. Cl. CPC A61K 31/519; A61K 31/5386; A61P 29/00; A61P 35/00; A61P 15/02; C07D 519/00

(57) ABSTRACT

The present invention relates to substituted imidazopyridinpyrimidine compounds of general formula (I) which inhibit MKNK1 and MKNK2 kinase (also known as MAP Kinase interacting Kinase, Mnk1 and Mnk2), to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyperproliferative, angiogenesis disorders, inflammatory diseases or diseases associated with inflammatory pain, as a sole agent or in combination with other active ingredients.

The present invention relates to substituted imidazopyridinpyrimidine compounds of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyperproliferative, angiogenesis disorders, inflammatory diseases or diseases associated with inflammatory pain, as a sole agent or in combination with other active ingredients.

### BACKGROUND OF THE INVENTION

The present invention relates to chemical compounds that inhibit MKNK1 kinase (also known as MAP Kinase interacting Kinase, Mnk1) and/or MKNK2 kinase (also known as MAP Kinase interacting Kinase, Mnk2). Human MKNKs comprise a group of four proteins encoded by two genes (Gene symbols: MKNK1 and MKNK2) by alternative splicing. The b-forms lack a MAP kinase-binding domain situated at the C-terminus. The catalytic domains of the MKNK1 and MKNK2 are very similar and contain a unique DFD (Asp-Phe-Asp) motif in subdomain VII, which usually is DFG (Asp-Phe-Gly) in other protein kinases and suggested to alter ATP binding [Jauch et al., Structure 13, 1559-1568, 2005 and Jauch et al., EMBO J25, 4020-4032, 2006]. MKNK1a binds to and is activated by ERK and p38 MAP Kinases, but not by JNK1. MKNK2a binds to and is activated only by ERK. MKNK1 b has low activity under all conditions and MKNK2b has a basal activity independent of ERK or p38 MAP Kinase. [Buxade M et al., Frontiers in Bioscience 5359-5374, May 1, 2008]

MKNKs have been shown to phosphorylate eukaryotic initiation factor 4E (eIF4E), heterogeneous nuclear RNA-binding protein A1 (hnRNP A1), polypyrimidine-tract binding protein-associated splicing factor (PSF), cytoplasmic phospholipase A2 (cPLA2) and Sprouty 2 (hSPRY2) [Buxade M et al., Frontiers in Bioscience 5359-5374, May 1, 2008].

eIF4E is an oncogene that is amplified in many cancers and is phosphorylated exclusively by MKNKs proteins as shown by KO-mouse studies [Konicek et al., Cell Cycle 7:16, 2466-2471, 2008; Ueda et al., Mol Cell Biol 24, 6539-6549, 2004]. eIF4E has a pivotal role in enabling the translation of cellular mRNAs. eIF4E binds the 7-methylguanosine cap at the 5′ end of cellular mRNAs and delivers them to the ribosome as part of the eIF4F complex, also containing eIF4G and eIF4A. Though all capped mRNAs require eIF4E for translation, a pool of mRNAs is exceptionally dependent on elevated eIF4E activity for translation. These so-called “weak mRNAs” are usually less efficiently translated due to their long and complex 5′UTR region and they encode proteins that play significant roles in all aspects of malignancy including VEGF, FGF-2, c-Myc, cyclin D1, survivin, BCL-2, MCL-1, MMP-9, heparanase, etc. Expression and function of eIF4E is elevated in multiple human cancers and directly related to disease progression [Konicek et al., Cell Cycle 7:16, 2466-2471, 2008].

MKNK1 and MKNK2 are the only kinases known to phosphorylate eIF4E at Ser209. Overall translation rates are not affected by eIF4E phosphorylation, but it has been suggested that eIF4E phosphorylation contributes to polysome formation (i.e. multiple ribosome on a single mRNA) that ultimately enables more efficient translation of “weak mRNAs” [Buxade M et al., Frontiers in Bioscience 5359-5374, May 1, 2008]. Alternatively, phosphorylation of eIF4E by MKNK proteins might facilitate eIF4E release from the 5′ cap so that the 48S complex can move along the “weak mRNA” in order to locate the start codon [Blagden S P and Willis A E, Nat Rev Clin Oncol. 8(5):280-91, 2011]. Accordingly, increased eIF4E phosphorylation predicts poor prognosis in non-small cell lung cancer patients [Yoshizawa et al., Clin Cancer Res. 16(1):240-8, 2010]. Further data point to a functional role of MKNK1 in carcinogenesis, as overexpression of constitutively active MKNK1, but not of kinase-dead MKNK1, in mouse embryo fibroblasts accelerates tumor formation [Chrestensen C. A. et al., Genes Cells 12, 1133-1140, 2007]. Moreover, increased phosphorylation and activity of MKNK proteins correlate with overexpression of HER2 in breast cancer [Chrestensen, C. A. et al., J. Biol. Chem. 282, 4243-4252, 2007]. Constitutively active, but not kinase-dead, MKNK1 also accelerated tumor growth in a model using Eμ-Myc transgenic hematopoietic stem cells to produce tumors in mice. Comparable results were achieved when an eIF4E carrying a S209D mutation was analyzed. The S209D mutation mimicks a phosphorylation at the MKNK1 phosphorylation site. In contrast, a non-phosphorylatable form of eIF4E attenuated tumor growth [Wendel H G, et al., Genes Dev. 21(24):3232-7, 2007]. A selective MKNK inhibitor that blocks eIF4E phosphorylation induces apoptosis and suppresses proliferation and soft agar growth of cancer cells in vitro. This inhibitor also suppresses outgrowth of experimental B16 melanoma pulmonary metastases and growth of subcutaneous HCT116 colon carcinoma xenograft tumors without affecting body weight [Konicek et al., Cancer Res. 71(5):1849-57, 2011]. In summary, eIF4E phosphorylation through MKNK protein activity can promote cellular proliferation and survival and is critical for malignant transformation. Inhibition of MKNK activity may provide a tractable cancer therapeutic approach.

Furthermore it has been found that MKNK1 is an acinar cell-specific kinase required for exocrine pancreatic secretion [Cendrowski J, Sánchez-Arévalo Lobo V J, Sendler M, et al. Gut Published Online First: Jul. 18, 2014; doi:10.1136/gutjnl-2013-306068].

The kinases MKNK1 and MKNK2 are important downstream targets of the Erk and p38 mitogen-activated protein kinase (MAPK) pathways and their activity can also be modulated by MAPK independent signals. The MKNKs are directly involved in regulating mRNA translation and, therefore, are key mediators of oncogenic progression and cytokine signaling. In particular, MAPK pathways such as Erk and p38 have been shown to play important roles in modulating immune responses by mediating the production of cytokines that control the initiation of innate immunity; the activation of adaptive immunity; and by regulating cellular responses to cytokines involved in immune responses. In addition, Erk and p38 contribute to pain sensitivity and p38 kinase inhibitors have shown pre-clinical and clinical efficacy regarding pain [Brown, Heitmeyer, et al., J Inflamm (Lond), 2008; Hill, Dabbagh, et al., J Pharmacol Exp TherJi, 2008; Gereau, et al., Brain Res Rev, 2009; Cheng, Dauch, et al., Mol Pain, 2010; Anand, Shenoy, et al., European Journal of Pain, 2011; Daves, Aitchison, et al., American College of Rheumatology Annual Meeting, 2012; Lin, Wang, et al., Curr Med Chem, 2014]. As MKNK kinases are effectors of MAPK pathways, these observations suggest that they may play important roles in mediating cytokine production and inflammatory pain. Recent studies support the involvement of MKNK kinases in different inflammatory processes [Rowlett, Chrestensen, et al., Am J Physiol Gastrointest Liver Physiol, 2008; Kjellerup, Kragballe, et al., Experimental Dermatology, 2008; Melemedjian, Asiedu, et al., J Neurosci, 2010; Fortin, Mayer, et al., Journal of Leukocyte Biolog, 2013]. Due to the induction of MKNK kinases by different inflammatory stimuli (sterile inflammation and pathogens) and their ability to regulate the expression of different cytokines which mediate the pathogenesis of multiple disorders such as auto-immune diseases, allergies, neurological disorders, sepsis, cardiovascular diseases, metabolic diseases, obesity and cancer. MKNKs represent a central node in regulating inflammation. [Joshi et al.; World J Biol Chem 2014 Aug. 26; 5(3): 321-333; Joschi et al., Biomol Concepts. 2012 April; 3(2): 127-139]

Imbalance in cytokines from Interleukin-1 family and their role in the pathogenesis of Endometriosis has been reported in the literature [American Journal of Reproductive Immunology 68 (2012) 138-145] as well as the possible pathophysiological roles of Mitogen-Activated Protein Kinases (MAPKs) in Endometriosis [Yoshino et al.; AJRI 2004; 52: 306-311]. More recently, the role of pro-inflammatory cytokines for evaluation of inflammatory status and their pathogenetic mechanisms in endometriosis has been illustrated [Tosti et al.; Reproductive Sciences 2015, 1-7; Malutan et al., Centr Eur J Immunol 2015; 40 (1): 96-102; Soo Hyun Ahn et al., BioMed Research International, Vol. 2015, Article ID 795976, 12 pages]. Women with endometriosis have elevated levels of key pro-inflammatory cytokines, i.e. IL-1β, IL-6, and TNF-α. At the same time, IL-11 and IL-6 could be used as predictors for endometriosis.

Substituted imidazopyridinpyrimidine compounds of general formula (I) have not been disclosed in prior art for the treatment or prophylaxis of different diseases.

So, the state of the art described above does not describe the specific substituted imidazopyridinpyrimidine compounds of general formula (I) of the present invention as defined herein or a stereoisomer, a tautomer, an N-oxide, a hydrate, a solvate, or a salt thereof, or a mixture of same, as described and defined herein, and as hereinafter referred to as “compounds of the present invention”, or their pharmacological activity.

It has now been found, and this constitutes the basis of the present invention, that said compounds of the present invention have surprising and advantageous properties.

In particular, said compounds of the present invention have been found to effectively inhibit MKNK1 kinase.

Furthermore, the compounds according to the present invention have been found to effectively inhibit MKNK2 kinase.

In contrast to other MKNK1 and/or MKNK2 kinase inhibitors, the imidazopyridinpyrimidines according to the invention are mainly active on sterile and pathogenic inflammatory responses and do not interfere directly with cell viability.

The imidazopyridinpyrimidines according to the present invention may be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1 and/or MKNK2 kinase, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The imidazopyridinpyrimidines according to the present invention may be used for the treatment or prophylaxis of inflammatory and/or immunological diseases as described in the summary of the invention.

Furthermore, the compounds according to the invention may be used for the treatment or prophylaxis of a gynecological disease, preferably dysmenorrhea, dyspareunia or endometriosis, adenomyosis, endometriosis-associated pain, or other endometriosis-associated symptoms, wherein said symptoms are in particular endometriosis-associated proliferation, dysmenorrhea, dyspareunia, dysuria, or dyschezia.

### SUMMARY OF THE INVENTION

The present invention covers compounds of general formula (I)

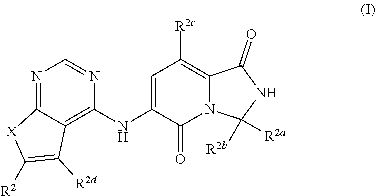

in which:
X represents NH, O or S;
R², R^{2d} together represent an C₃-C₅-alkylene group said group being optionally substituted, identically or differently, with 1, 2, or 3 R⁵-groups which represent hydrogen, halogen, cyano, hydroxy, a group —C₁-C₃-alkyl, —C₁-C₃-alkyl-OH,
   —C₁-C₆-alkoxy, —(C═O)—C₁-C₆-alkyl, —(C═O)—C₃-C₆-cycloalkyl, —C(C═O)—OH, —C(C═O)—O—C₁-C₆-alkyl, —NH₂, —N(H)—C₁-C₃-alkyl, —N(—C₁-C₃-alkyl)-C₁-C₃-alkyl, —(C═O)—N(H)—C₁-C₆-alkyl, —(C═O)—N(—C₁-C₆-alkyl)-C₁-C₆-alkyl, —NH(C═O)—O—C₁-C₆-alkyl; or
   said C₃-C₅-alkylene group is substituted one time with R¹ which represents a hydrogen atom or a group —CO₂—C₁-C₆-alkyl, —CO₂H, —C(═O)N(R³)R⁴;
R^{2a}, R^{2b} represent independently from each other a group selected from hydrogen, C₁-C₆-alkyl-;
or
C(R^{2a})R^{2b} together
   represent a group C(═O) or a 3- to 8-membered cycloalkyl-, 4- to 8-membered heterocycloalkyl- group, wherein said 3- to 8-membered cycloalkyl-, 4- to 8-membered heterocycloalkyl- group is optionally substituted one, two or three times, identically or differently, with a halogen atom or a group selected from: —OR⁸, —N(R⁸)R⁹, —CN, —C(═O)N(R⁸)R⁹, C₁-C₃-alkyl-, C₁-C₃-haloalkyl-, C₁-C₆-alkoxy-, C₁-C₆-haloalkoxy-, aryl, —(CH₂)_{q}-heteroaryl, —(CH₂)_{q}—N(R⁸)R⁹;
R^{2c} represents a hydrogen or a halogen atom or a C₁-C₄-alkyl- group, wherein said C₁-C₄-alkyl- group is optionally substituted one, two or three times, identically or differently, with a halogen atom or a group selected from: —OR⁸, —N(R⁷)R⁸;
R³ represents a hydrogen atom or a C₁-C₆-alkyl-, group, wherein said C₁-C₆-alkyl- group is optionally substituted one, two or three times, identically or differently, with a group selected from halo-, C₁-C₃-alkoxy-, HO—, —N(R⁸)R⁹;
R⁴ represents a hydrogen atom or a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterospirocycloalkyl group;
   wherein said 3- to 10-membered heterocycloalkyl- group is optionally substituted one, two or three times, identically or differently, with a halogen atom or a group selected from: C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, —(C₁-C₃-alkyl)-N(R⁸)R⁹;
R⁷ represents a C₁-C₄-alkyl- or a —C(═O)—O—C₁-C₆-alkyl group;
   wherein said C₁-C₄-alkyl- group is optionally substituted once with —OH or —N(R⁸)R⁹;
R⁸ represents a hydrogen atom or a C₁-C₄-alkyl- group;
R⁹ represents a hydrogen atom or a C₁-C₆-alkyl- group;
q represents an integer of 0, 1, 2 or 3;
or a tautomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings: The term “halogen atom”, “halo-” or “Hal-” is to be understood as meaning a fluorine, chlorine, bromine or iodine atom, preferably a fluorine, chlorine or bromine atom.

The term “C₁-C₁₀-alkyl” is to be understood as preferably meaning a linear or branched, saturated, monovalent hydrocarbon group having 1, 2, 3, 4, 5, 6, 7, 8, 9 or 10 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3, 4, 5 or 6 carbon atoms (“C₁-C₆-alkyl”), more particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group; even more particularly 1, 2 or 3 carbon atoms (“C₁-C₃-alkyl”), e.g. a methyl, ethyl, n-propyl- or iso-propyl group.

The term “C₁-C₁₀-alkylene” is to be understood as preferably meaning a linear or branched, saturated, bivalent hydrocarbon group having 1, 2, 3, 4, 5, 6, 7, 8, 9 or 10 carbon atoms, e.g. a methylene, ethylene, n-propylene, n-butylene, n-pentylene, 2-methylbutylene, n-hexylene, 3-methylpentylene group, or an isomer thereof. Particularly, said group is linear and has 2, 3, 4 or 5 carbon atoms (“C₂-C₅-alkylene”), e.g. an ethylene, n-propylene, n-butylene, n-pentylene group, more particularly 3 or 4 carbon atoms (“C₃-C₄-alkylene”), e.g. an n-propylene or n-butylene group.

The term “halo-C₁-C₆-alkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said halo-C₁-C₆-alkyl group is, for example, —CF₃, —CHF₂, —CH₂F, —CF₂CF₃ or —CH₂CF₃.

The term “C₁-C₆-alkoxy” is to be understood as preferably meaning a linear or branched, saturated, monovalent, hydrocarbon group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃ or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃ or —CH₂CH₂OCH₂CF₃.

The term “C₂-C₁₀-alkenyl” is to be understood as preferably meaning a linear or branched, monovalent hydrocarbon group, which contains one or more double bonds, and which has 2, 3, 4, 5, 6, 7, 8, 9 or 10 carbon atoms, particularly 2, 3, 4, 5 or 6 carbon atoms (“C₂-C₆-alkenyl”), more particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-propylprop-1-enyl, (E)-2-isopropyl prop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₁₀-alkynyl” is to be understood as preferably meaning a linear or branched, monovalent hydrocarbon group which contains one or more triple bonds, and which contains 2, 3, 4, 5, 6, 7, 8, 9 or 10 carbon atoms, particularly 2, 3, 4, 5 or 6 carbon atoms (“C₂-C₆-alkynyl”), more particularly 2 or 3 carbon atoms (“C₂-C₃-alkynyl”). Said C₂-C₁₀-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C₃-C₆-cycloalkyl”).

The term “C₃-C₆-cycloalkyloxy” refers to a (C₃-C₆-cycloalkyl)-O— group in which “C₃-C₆-cycloalkyl” is as defined herein. Examples include, but are not limited to, cyclopropanoxy and cyclobutanoxy.

The term “C₄-C₁₀-cycloalkenyl” is to be understood as preferably meaning a non-aromatic, monovalent, mono- or bicyclic hydrocarbon ring which contains 4, 5, 6, 7, 8, 9 or 10 carbon atoms and one, two, three or four double bonds, in conjugation or not, as the size of said cycloalkenyl ring allows. Said C₄-C₁₀-cycloalkenyl group is for example, a monocyclic hydrocarbon ring, e.g. a cyclobutenyl, cyclopentenyl, or cyclohexenyl or a bicyclic hydrocarbon, e.g.:

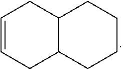

The term “C₅-C₈-cycloalkenyloxy” refers to a (C₅-C₈-cycloalkenyl)-O— group in which “C₅-C₈-cycloalkenyl” is as defined herein.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from —C(═O)—, —O—, —S—, —S(═O)—, —S(═O)₂—, —N(R^{a})—, in which R^{a} represents a hydrogen atom or a C₁-C₆-alkyl group; it being possible for said heterocycloalkyl group to be attached to the rest of the molecule via any one of the carbon atoms or, if present, the nitrogen atom. Heterospirocycloalkyl, heterobicycloalkyl and bridged heterocycloalkyl, as defined infra, are also included within the scope of this definition.

The term “heterospirocycloalkyl” is to be understood as meaning a saturated, monovalent bicyclic hydrocarbon radical in which the two rings share one common ring carbon atom, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl- group; it being possible for said heterospirocycloalkyl- group to be attached to the rest of the molecule via any one of the carbon atoms or, if present, the nitrogen atom. Said heterospirocycloalkyl-group is, for example, azaspiro[2.3]hexyl-, azaspiro[3.3]heptyl-, oxaazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, or azaspiro[5.5]decyl-.

The term “heterobicycloalkyl” is to be understood as meaning a saturated, monovalent bicyclic hydrocarbon radical in which the two rings share two immediately adjacent ring atoms, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl-group; it being possible for said heterobicycloalkyl- group to be attached to the rest of the molecule via any one of the carbon atoms or, if present, the nitrogen atom. Said heterobicycoalkyl- group is, for example, azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl-, or azabicyclo[4.4.0]decyl-.

The term “bridged heterocycloalkyl” is to be understood as meaning a saturated, monovalent bicyclic hydrocarbon radical in which the two rings share two common ring atoms which are not immediately adjacent, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom, or a C₁-C₆-alkyl- or C₃-C₇-cycloalkyl- group; it being possible for said bridged heterocycloalkyl-group to be attached to the rest of the molecule via any one of the carbon atoms or, if present, the nitrogen atom. Said bridged heterocycloalkyl- group is, for example, azabicyclo[2.2.1]heptyl-, oxazabicyclo[2.2.1]heptyl-, thiazabicyclo[2.2.1]heptyl-, diazabicyclo[2.2.1]heptyl-, azabicyclo[2.2.2]octyl-, diazabicyclo[2.2.2]octyl-, oxazabicyclo[2.2.2]octyl-, thiazabicyclo[2.2.2]octyl-, azabicyclo[3.2.1]octyl-, diazabicyclo[3.2.1]octyl-, oxazabicyclo[3.2.1]octyl-, thiazabicyclo[3.2.1]octyl-, azabicyclo[3.3.1]nonyl-, diazabicyclo[3.3.1]nonyl-, oxazabicyclo[3.3.1]nonyl-, thiazabicyclo[3.3.1]nonyl-, azabicyclo[4.2.1]nonyl-, diazabicyclo[4.2.1]nonyl-, oxazabicyclo[4.2.1]nonyl, thiazabicyclo[4.2.1]nonyl-, azabicyclo[3.3.2]decyl-, diazabicyclo[3.3.2]decyl-, oxazabicyclo[3.3.2]decyl-, thiazabicyclo[3.3.2]decyl-, or azabicyclo[4.2.2]decyl-.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said 3- to 10-membered heterocycloalkyl can contain 4 or 5 carbon atoms, and one or more of the above-mentioned heteroatom-containing groups (a “5- to 6-membered heterocycloalkyl”).

Particularly, without being limited thereto, said 3- to 10-membered heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

Said 3- to 10-membered heterocycloalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

The term “4- to 10-membered heterocycloalkenyl”, is to be understood as meaning an non-aromatic, unsaturated, monovalent, mono- or bicyclic hydrocarbon ring which contains 3, 4, 5, 6, 7, 8 or 9 carbon atoms, and one or more heteroatom-containing groups selected from —C(═O)—, —O—, —S—, —S(═O)—, —S(═O)₂—, —N(R^{a})—, in which R^{a} represents a hydrogen atom or a C₁-C₆-alkyl group; it being possible for said heterocycloalkenyl group to be attached to the rest of the molecule via any one of the carbon atoms or, if present, the nitrogen atom. Examples of said heterocycloalkenyl are e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic or partially aromatic, mono-, bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a biphenyl group, or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a ring having 13 carbon atoms, (a “C₁₃-aryl” group), e.g. a fluorenyl group, or a ring having 14 carbon atoms, (a “C₁₄-aryl” group), e.g. an anthranyl group. Preferably, the aryl group is a phenyl group.

The term “heteroaryl” is understood as preferably meaning a monovalent, monocyclic, bicyclic or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

As used herein, the term “protective group” is a protective group attached to a nitrogen in intermediates used for the preparation of compounds of the general formula (I). Such groups are introduced e.g. by chemical modification of the respective amino group in order to obtain chemoselectivity in a subsequent chemical reaction. Protective groups for amino groups are descibed for example in T. W. Greene and P. G. M. Wuts in Protective Groups in Organic Synthesis, 3^{rd} edition, Wiley 1999; more specifically, said groups can be selected from substituted sulfonyl groups, such as mesyl-, tosyl- or phenylsulfonyl-, acyl groups such as benzoyl, acetyl or tetrahydropyranoyl-, or carbamate based groups, such as tert.-butoxycarbonyl (Boc), or can include silicon, as in e.g. 2-(trimethylsilyl)ethoxymethyl (SEM).

The invention includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), 3H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention may contain sulphur atoms which are asymmetric, such as an asymmetric sulphoxide or sulphoximine group, of structure:

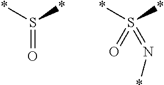

for example, in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

Pure stereoisomers can be obtained by resolution of racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R) or (S) isomers, or (E) or (Z) isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

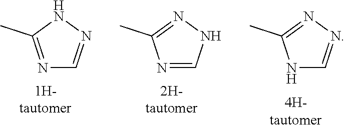

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol, or with a quarternary ammonium salt, such as tetramethylammonium, tetraethylammonium, tetra(n-propyl)ammonium, tetra (n-butyl)ammonium, or N-benzyl-N,N,N-trimethylammonium.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

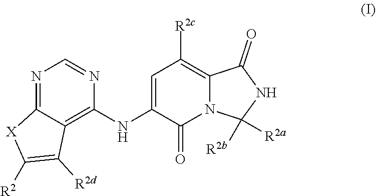

in which:
X represents NH, O or S;
R², R^{2d} together represent an C₃-C₅-alkylene group said group being optionally substituted, identically or differently, with 1, 2, or 3 R⁵-groups which represent hydrogen, halogen, cyano, hydroxy, a group —C₁-C₃-alkyl, —C₁-C₃-alkyl-OH,
   —C₁-C₆-alkoxy, —(C═O)—C₁-C₆-alkyl, —(C═O)—C₃-C₆-cycloalkyl, —C(C═O)—OH, —C(C═O)—O—C₁-C₆-alkyl, —NH₂, —N(H)—C₁-C₃-alkyl, —N(—C₁-C₃-alkyl)-C₁-C₃-alkyl, —(C═O)—N(H)—C₁-C₆-alkyl, —(C═O)—N(—C₁-C₆-alkyl)-C₁-C₆-alkyl, —NH(C═O)—O—C₁-C₆-alkyl; or
   said C₃-C₅-alkylene group is substituted one time with R¹ which represents a hydrogen atom or a group —CO₂—C₁-C₆-alkyl, —CO₂H, —C(═O)N(R³)R⁴;
R^{2a}, R^{2b} represent independently from each other a group selected from hydrogen, C₁-C₆-alkyl-;
or
C(R^{2a})R^{2b} together
   represent a group C(═O) or a 3- to 8-membered cycloalkyl-, 4- to 8-membered heterocycloalkyl- group, wherein said 3- to 8-membered cycloalkyl-, 4- to 8-membered heterocycloalkyl- group is optionally substituted one, two or three times, identically or differently, with a halogen atom or a group selected from: —OR⁸, —N(R⁸)R⁹, —CN, —C(═O)N(R⁸)R⁹, C₁-C₃-alkyl-, C₁-C₃-haloalkyl-, C₁-C₆-alkoxy-, C₁-C₆-haloalkoxy-, aryl, —(CH₂)_{q}-heteroaryl, —(CH₂)_{q}—N(R⁸)R⁹;
R^{2c} represents a hydrogen or a halogen atom or a C₁-C₄-alkyl- group, wherein said C₁-C₄-alkyl- group is optionally substituted one, two or three times, identically or differently, with a halogen atom or a group selected from: —OR⁸, —N(R⁷)R⁸;
R³ represents a hydrogen atom or a C₁-C₆-alkyl-, group, wherein said C₁-C₆-alkyl- group is optionally substituted one, two or three times, identically or differently, with a group selected from halo-, C₁-C₃-alkoxy-, HO—, —N(R⁸)R⁹;
R⁴ represents a hydrogen atom or a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterospirocycloalkyl group;
   wherein said 3- to 10-membered heterocycloalkyl- group is optionally substituted one, two or three times, identically or differently, with a halogen atom or a group selected from: C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, —(C₁-C₃-alkyl)-N(R⁸)R⁹;
R⁷ represents a C₁-C₄-alkyl- or a —C(═O)—O—C₁-C₆-alkyl group;
wherein said C₁-C₄-alkyl- group is optionally substituted once with —OH or —N(R⁸)R⁹;
R⁸ represents a hydrogen atom or a C₁-C₄-alkyl- group;
R⁹ represents a hydrogen atom or a C₁-C₆-alkyl- group;
q represents an integer of 0, 1, 2 or 3;
or a tautomer, an N-oxide, a hydrate, a solvate, or a salt thereof, or a mixture of same.

A further aspect of the present invention refers to the compounds of general formula (Ia)

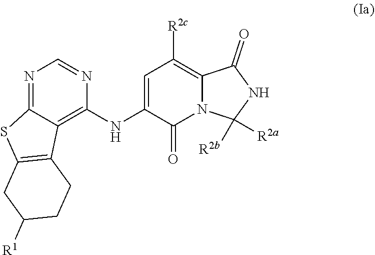

in which:
R¹ represents a hydrogen atom or a group —CO₂—C₁-C₆-alkyl, —CO₂H, —C(═O)N(R³)R⁴;
R^{2a}, R^{2b} represent independently of each other a group selected from hydrogen, C₁-C₆-alkyl-; or
C(R^{2a})R^{2b} together
   represent a group C(═O) or a 3- to 8-membered cycloalkyl-, 4- to 8-membered heterocycloalkyl- group,
   wherein said 3- to 8-membered cycloalkyl-, 4- to 8-membered heterocycloalkyl- group is optionally substituted one, two or three times, identically or differently, with a halogen atom or a group selected from: —OR⁸, —N(R⁸)R⁹, —CN, —C(═O)N(R⁸)R⁹, C₁-C₃-alkyl-, C₁-C₃-haloalkyl-, C₁-C₆-alkoxy-, C₁-C₆-haloalkoxy-, aryl, —(CH₂)_{q}-heteroaryl, —(CH₂)_{q}—N(R⁸)R⁹;
R^{2c} represents a hydrogen or a halogen atom or a C₁-C₄-alkyl- group wherein said a C₁-C₄-alkyl- group is optionally substituted one, two or three times, identically or differently, with a halogen atom or a group selected from: —OR⁸, —N(R⁷)R⁸;
R³ represents a hydrogen atom or a C₁-C₆-alkyl-, group, wherein said C₁-C₆-alkyl- group is optionally substituted one, two or three times, identically or differently, with a group selected from: halo-, C₁-C₃-alkoxy-, HO—, —N(R⁸)R⁹;
R⁴ represents a hydrogen atom or a C₁-C₄-alkyl- group,
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group,
   wherein said 3- to 10-membered heterocycloalkyl- group is optionally substituted one, two or three times, identically or differently, with a halogen atom or a group selected from: C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, —(C₁-C₃-alkyl)-N(R⁸)R⁹;
R⁷ represents a C₁-C₄-alkyl-, or a —C(═O)—O—C₁-C₆-alkyl group; wherein said C₁-C₄-alkyl- group is optionally substituted once with —OH or —N(R⁸)R⁹;
R⁸ represents a hydrogen atom or a C₁-C₄-alkyl- group;
R⁹ represents a hydrogen atom or a C₁-C₆-alkyl- group;
q represents an integer of 0, 1, 2 or 3;
or a tautomer, an N-oxide, a hydrate, a solvate, or a salt thereof, or a mixture of same.

A further aspect of the present invention refers to the compounds of general formula (Ib):

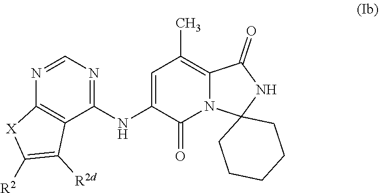

in which
X represents NH, O or S;
R², R^{2d} together represent an C₃-C₅-alkylene group;
   said group being optionally substituted, identically or differently, with 1, 2, or 3 R⁸-groups;
R⁸ represents hydrogen, halogen, cyano, hydroxy, a group —C₁-C₃-alkyl, —C₁-C₃-alkyl-OH, —C₁-C₆-alkoxy, —(C═O)—C₁-C₆-alkyl, —(C═O)—C₃-C₆-cycloalkyl, —C(C═O)—OH, —C(C═O)—O—C₁-C₆-alkyl, —NH₂, —N(H)—C₁-C₃-alkyl, —N(—C₁-C₃-alkyl)-C₁-C₃-alkyl, —(C═O)—N(H)—C₁-C₆-alkyl, —(C═O)—N(—C₁-C₆-alkyl)-C₁-C₆-alkyl, —NH(C═O)—O—C₁-C₆-alkyl;
or a tautomer, an N oxide, a hydrate, a solvate, a salt thereof, or a mixture of same.

In another preferred embodiment, the invention relates to compounds of formula (Ia):

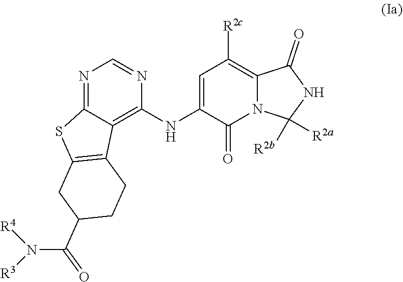

in which R^{2a}, R^{2b}, R^{2c}, R³ and R⁴ are as defined for compounds of formula (I).

In another preferred embodiment, the invention relates to compounds of formula (Ia) with defined sterochemistry:

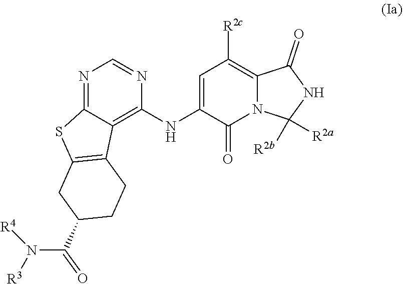

in which R^{2a}, R^{2b}, R^{2c}, R³ and R⁴ are as defined for compounds of formula (I).

In another preferred embodiment, the invention relates to compounds of formula I, Ia, Ib supra, X represents S.

In another preferred embodiment, the invention relates to compounds of formula (I), (Ia), (Ib) supra, R^{2a}, R^{2b} represent independently from each other a group selected from hydrogen, fluor, methy, or CF₃; more in particular hydrogen or methyl.

In another preferred embodiment, the invention relates to compounds of formula I, Ia, Ib supra, C(R^{2a})R^{2b} together represent —(CH₂—CH_{2—})—, —(CH₂—CR₆(R₆ₐ)—CH_{2—})—, —(CH₂—Z—CH_{2—})—, —(CH₂—CH₂—CR₁₁(R₁₁ₐ)—CH_{2—})—, —(CH₂—CH₂—CR₁₁(R₁₁ₐ)—CH₂—CH_{2—})—; and Z represents NR⁷, O, S, S(═O) or SO₂; more particularly C(R^{2a})R^{2b} together represent represent —(CH₂—CH_{2—})—, —(CH₂—CH₂—CH_{2—})—, —(CH₂—S—CH_{2—})—, —(CH₂—S(O)₂—CH_{2—})—, —(CH₂—CH₂—CH₂—CH_{2—})—, —(CH₂—CH₂—CH₂—CH₂—CH_{2—})—, —(CH₂—CH₂—CF₂—CH₂—CH_{2—})—, —(CH₂—CH₂—C(CF₃)OH—CH₂—CH_{2—})—, —(CH₂—CH₂—C(CF₂CF₃)OH—CH₂—CH_{2—})—.

In another preferred embodiment, the invention relates to compounds of formula (I), (Ia), (Ib) supra, R^{2c} represents a chlor or methyl, in particular methyl.

In another preferred embodiment, the invention relates to compounds of formula (I), or (Ib) supra in which R², R^{2d} together represent a group —(CH₂—CH(C(O)OH)—CH₂—CH₂)—, —(CH₂—CH(C(O)NH₂)—CH₂—CH₂)—.

In another preferred embodiment, the invention relates to compounds of formula (I) or (Ia), supra, wherein N(R³)R⁴ represent a group N(CH₃)CH₃:

In another preferred embodiment, the invention relates to compounds of formula (I) or (Ia), supra, wherein N(R³)R⁴ together represent a group selected from:

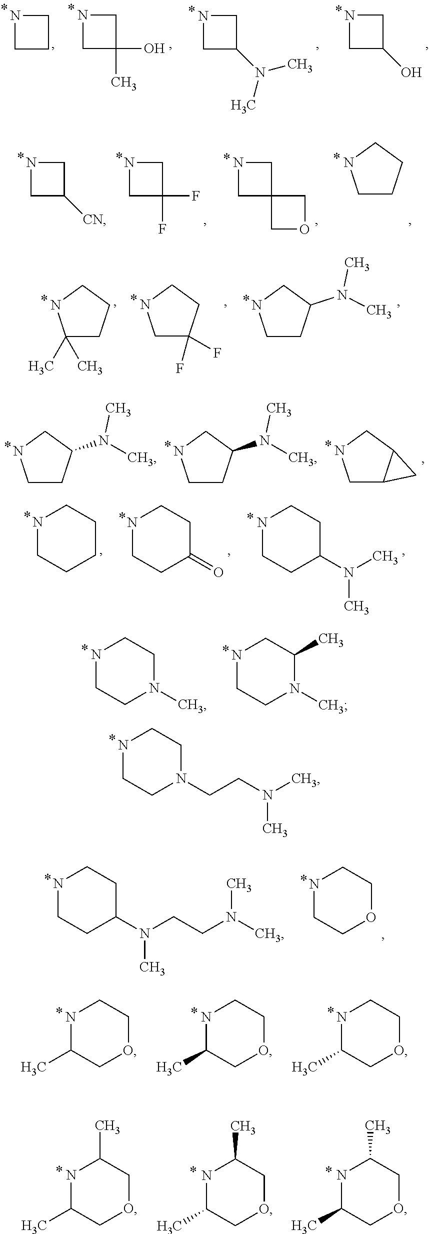

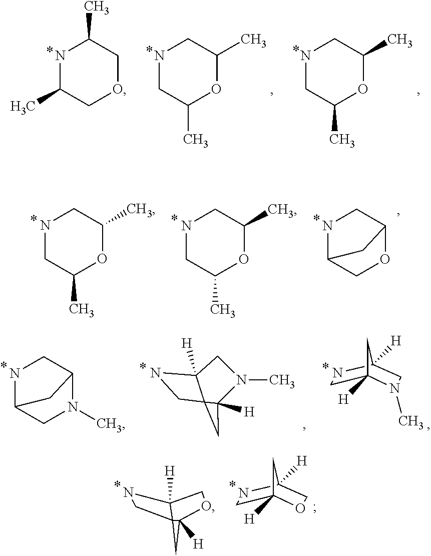

wherein * indicates the point of attachment of said groups with the rest of the molecule.

In another preferred embodiment, the invention relates to compounds of formula (I) or (Ia), supra, wherein N(R³)R⁴ together represent a group selected from:

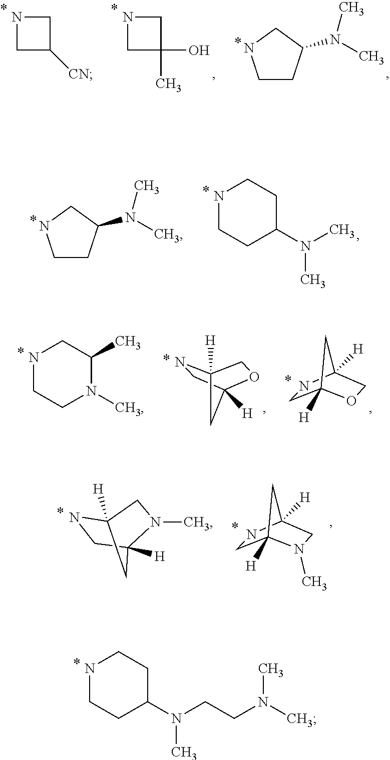

wherein * indicates the point of attachment of said groups with the rest of the molecule.

It is to be understood that the present invention relates also to any combination of the preferred embodiments described above. Some examples of combinations are given hereinafter. However, the invention is not limited to these combinations.

In another preferred embodiment, the invention relates to compounds of formula (Ia) with defined sterochemistry:

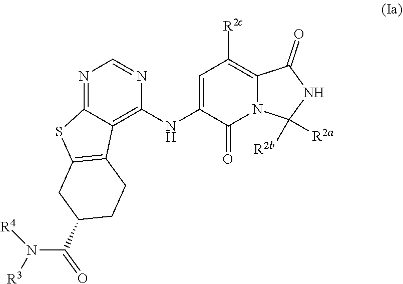

in which:
R^{2a}, R^{2b} represent independently of each other a group selected from hydrogen, methyl, or fluor; or
C(R^{2a})R^{2b} together represent —(CH₂—CH_{2—})—, —(CH₂—CR₆(R₆ₐ)—CH_{2—})—, —(CH₂—Z—CH_{2—})—, —(CH₂—CH₂—CR₁₁(R₁₁ₐ)—CH_{2—})—, —(CH₂—CH₂—CR₁₁(R₁₁ₐ)—CH₂—CH_{2—})—; and
   Z represents NR⁷, O, S, S(═O) or SO₂;
R^{2c} represents a hydrogen, fluor, chlor or methyl;
R³ represents a hydrogen atom or a C₁-C₆-alkyl-, group, wherein said C₁-C₆-alkyl- group is optionally substituted one, two or three times, identically or differently, with a group selected from: halo-, C₁-C₃-alkoxy-, HO—, —N(R⁸)R⁹;
R⁴ represents a hydrogen atom or a C₁-C₄-alkyl- group, wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group,
   wherein said 3- to 10-membered heterocycloalkyl- group is optionally substituted one, two or three times, identically or differently, with a halogen atom or a group selected from: C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, —(C₁-C₃-alkyl)-N(R⁸)R⁹;
R⁶, R^{6a} represent independently from one another a hydrogen atom, fluor or a C₁-C₄-alkyl- group;
R⁷ represents a C₁-C₄-alkyl-, or a —C(═O)—O—C₁-C₆-alkyl group; wherein said C₁-C₄-alkyl- group is optionally substituted once with —OH or —N(R⁸)R⁹;
R⁸ represents a hydrogen atom or a C₁-C₄-alkyl- group;
R⁹ represents a hydrogen atom or a C₁-C₆-alkyl- group;
q represents an integer of 0, 1, 2 or 3;
or a tautomer, an N-oxide, a hydrate, a solvate, or a salt thereof, or a mixture of same.

In another preferred embodiment, the invention relates to compounds of formula (Ia) with defined sterochemistry

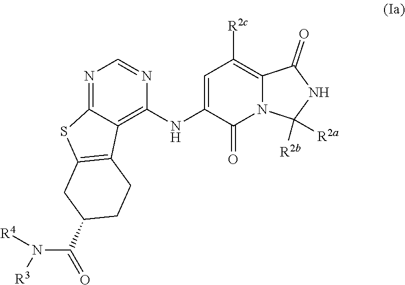

in which:
R^{2a}, R^{2b} represent independently of each other a group selected from hydrogen, methyl, or fluor;
or
C(R^{2a})R^{2b} together represent —(CH₂—CH_{2—})—, —(CH₂—CR₆(R₆ₐ)—CH_{2—})—, —(CH₂—Z—CH_{2—})—, —(CH₂—CH₂—CR₁₁(R₁₁ₐ)—CH_{2—})—, —(CH₂—CH₂—CR₁₁(R₁₁ₐ)—CH₂—CH_{2—})—; and
   Z represents NR⁷, O, S, S(═O) or SO₂;
R^{2c} represents a methyl;
N(R³)R⁴ represent a group N(CH₃)CH₃ or
N(R³)R⁴ together represent a group selected from:

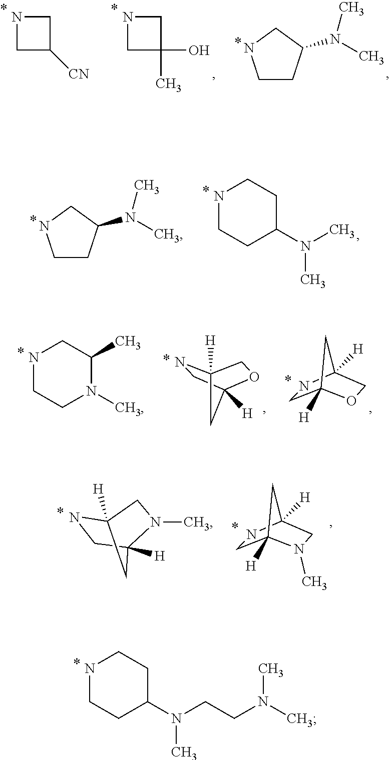

wherein * indicates the point of attachment of said groups with the rest of the molecule;
R¹¹ and R^{11a} represent independently of each other hydrogen, fluor, hydroxy, methyl or CF₃;
or a tautomer, an N-oxide, a hydrate, a solvate, or a salt thereof, or a mixture of same.

In another preferred embodiment, the invention relates to compounds of formula (Ia) with defined sterochemistry

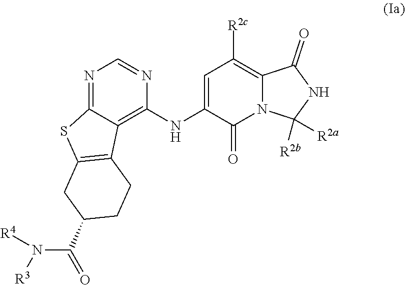

in which:
C(R^{2a})R^{2b} together represent —(CH₂—S—CH_{2—})—, —(CH₂—S(O)₂—CH_{2—})—, —(CH₂—CH₂—CH₂—CH_{2—})—, —(CH₂—CH₂—CH₂—CH₂—CH_{2—})—, —(CH₂—CH₂—CF₂—CH₂—CH_{2—})—, —(CH₂—CH₂—C(CF₃) OH—CH₂—CH_{2—})—, —(CH₂—CH₂—C(CF₂CF₃)OH—CH₂—CH_{2—})—,
R^{2c} represents a methyl;
N(R³)R⁴ represent a group N(CH₃)CH₃, or
N(R³)R⁴ together represent a group selected from:

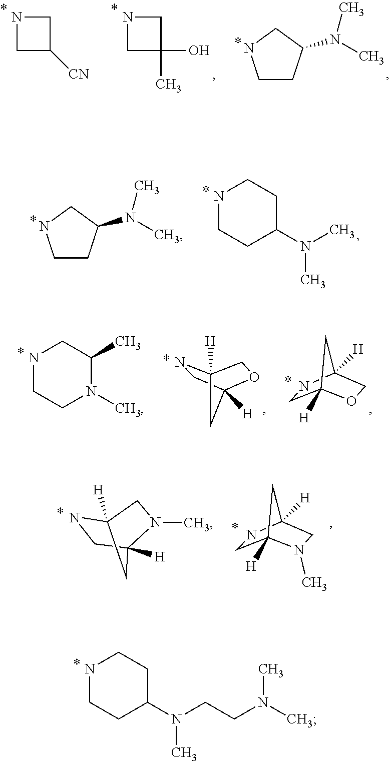

wherein * indicates the point of attachment of said groups with the rest of the molecule; or a tautomer, an N-oxide, a hydrate, a solvate, or a salt thereof, or a mixture of same.

It is to be understood that the present invention relates to any sub-combination within any embodiment or aspect of the present invention of compounds of general formula (I), supra.

More particularly still, the present invention covers compounds of general formula (I) which are disclosed in the Examples section of this text, infra.

In accordance with another aspect, the present invention covers methods of preparing compounds of the present invention, said methods comprising the steps as described in the Experimental Section herein.

In a preferred embodiment, the present invention relates to a method of preparing compounds of general formula (I), supra, in which method an intermediate compound of general formula (III):

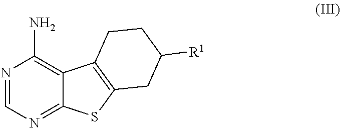

in which R¹ is as defined for general formula (I), supra,+is allowed to react with an intermediate compound of general formula (V):

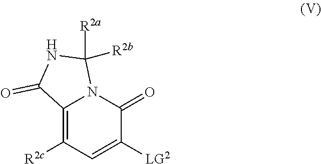

in which R^{2a}, R^{2b} and R^{2c} are as defined for general formula (I), supra, and LG² represents a leaving group LG.

In accordance with a further aspect, the present invention covers intermediate compounds which are useful in the preparation of compounds of the present invention of general formula (I), particularly in the method described herein. In particular, the present invention covers compounds of general formula (II):

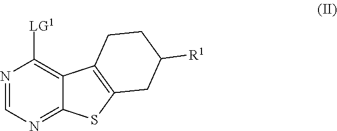

in which R¹ is as defined for the compounds of general formula (I), supra, and LG¹ represents a leaving group LG, preferably a chlorine atom.

Synthesis of Compounds of General Formula (I) of the Present Invention

Compounds of general formula (I) can be synthesized according to the general procedure depicted in Scheme 1, wherein LG¹ and LG² stand for a leaving group LG.

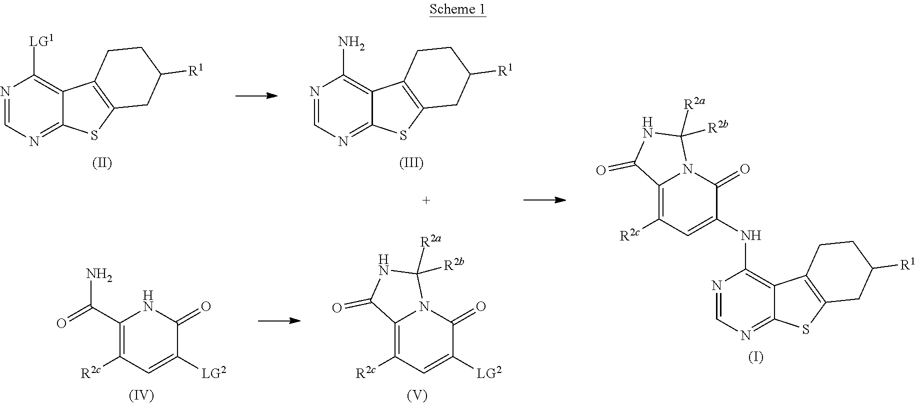

Scheme 1 exemplifies the main route that allows variations in R¹, R^{2a}, R^{2b} and R^{2c}. The coupling of compounds of formula (III) with compounds of formula (V) can be accomplished by reacting the two reactants in a suitable solvent, such as ethanol or a related lower aliphatic alcohol of the formula C₁-C₄-alkyl-OH or a cyclic ether, such as tetrahydrofuran or 1,4-dioxane, or N,N-dimethylacetamide or N,N-dimethylformamide. The compounds of formula (III) can be prepared from compounds auf formula (II) by replacing the group LG¹ with ammonia, preferentially by using an aqueous ammonia solution under elevated temperature and pressure. The compounds of formula (III) can be used either as free base or as corresponding salt with organic or inorganic acids. Preferentially, palladium catalysed amination reactions can be employed to form compounds of general formula (I) from compounds of formulae (II) with (V); for a contemporary review on such aminations see e.g. David S. Surry and Stephen L Buchwald, Chem. Sci. 2011, 2, 27, or J. Y. Yoon et al., Synthesis 2009, (5), 815, and the literature cited therein.

Compounds of general formula (I) can also be built by Ullmann-type coupling reactions in the presence of suitable catalysts, such as, for example, copper based catalysts like copper(II)diacetate or copper(I)chloride in the presence of a suitable base, like for example, caesium carbonate starting from compounds of general formulae (III) and (V). Optionally, suitable ligands like N,N-dimethylglycine or phenyl hydrogen pyrrolidin-2-ylphosphonate can be added. The reaction can be performed at temperatures ranging from −40° C. to the boiling point of the solvent, for example.

Modification of any of the substituents, R¹, R^{2a}, R^{2b} and R^{2c} can be achieved before and/or after the exemplified transformation. However, also other routes may be used to synthesize the target compounds, in accordance with common general knowledge of a person skilled in the art of organic synthesis.

Said modifications can be such as the introduction of protecting groups, cleavage of protecting groups, reduction or oxidation of functional groups, formation or cleavage of esters or carboxamides, halogenation, metallation, substitution or other reactions known to a person skilled in the art. These transformations include those which introduce a functionality which allows for further interconversion of substituents. Appropriate protecting groups and their introduction and cleavage are well-known to a person skilled in the art (see for example T. W. Greene and P. G. M. Wuts in Protective Groups in Organic Synthesis, 3^{rd} edition, Wiley 1999).

Further, it is possible that two or more successive steps may be performed without work-up being performed between said steps, e.g. a “one-pot” reaction, as it is well-known to a person skilled in the art.

Compounds of the general formula (II), wherein R¹ has the meaning as given for general formula (I), can be readily prepared as shown in Scheme 2 by a so-called Gewald thiophene synthesis (for a seminal publication see e.g. K. Gewald et al., Chem. Ber. 1966, 94, 99), starting from ketones of the general formula (VI), to give the intermediate thiophene derivatives (VII). Said intermediates are then cyclised to the thienopyrimidones (VIII) employing a suitable C₁ synthon such as formamide. The resulting pyrimidones (VIII) are then transferred into compounds of the general formula (II) by suitable procedures known to the person skilled in the art, such as treatment with a chlorinating agent. An instructive exemplary protocol for the sequence outlined in Scheme 2 can be found in WO 2005/010008, example 14, steps 1 to 3.

If R¹ in compounds of the formula (II) comprises a carboxylic ester, e.g. an ethyl ester, it is well possible to convert said ester into a carboxamide in the presence of LG¹ e.g. representing a chloride, by mild ester hydrolysis using e.g. lithium hydroxide, followed by carboxamide coupling by procedures well known to the person skilled in the art.

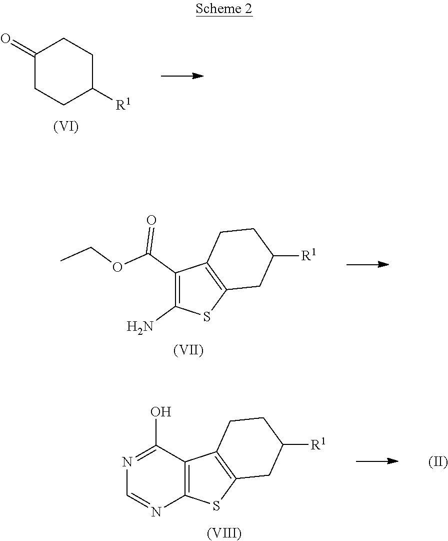

Multiple methods of isolating pure enantiomers from isomeric mixtures, e.g. racemic mixtures of chiral compounds are known to the person skilled in the art. Said methods encompass preparative HPLC on chiral stationary phase, kinetic resolution of racemic mixtures (for some examples see e.g. I. Shiina et al., Catal. Sci Technol. 2012, 2, 2200-2205; I. Shiina et al., Eur. J. Org. Chem. 2008, 5887-5890; D. G. Walker et al., Organic Process Research & Development 2001, 5, 23-27; B. N. Roy et al., Organic Process Research & Development 2009, 13, 450; T. Storz and P. Dittmar, Organic Process Research & Development 2003, 7, 559), enantioselective protonation (for some examples see e.g. C. Fehr and G. Galindo, Helv. Chim. Acta 1995, 78, 539-552, S. Hünig et al., Chem. Ber. 1994, 127, 1981-1988; S. Hünig et al., Chem. Ber. 1994, 127, 1969), enzymatic resolution (for some examples see e.g. T. Miyazawa, Amino Acids 1999, 16, 191-213), or temporary derivatisation with an enantiopure chiral synthon, separation of the resulting diastereomers, and removal of said chiral synthon, resulting in the isolation of the pure enantiomers of the parent compound (for some examples see e.g. Asymmetric Synthesis—The Essentials. Edited by Mathias Christmann and Stefan Brase WILEY-VCH Verlag GmbH & Co. KGaA, Weinheim). Instructive exemplary protocols for preparation of enantiomerically pure compounds of the formula (II) can be found in WO 2015/074986, example 1, steps 1c to 1f.

Instructive exemplary protocols for the preparation of compounds of formulae (IV) and (V) can be found in WO2015/200481.

For example, further elaboration of compounds of formulae (I), (II), (Ill), (VI), (VII) or (VIII) in which R¹ stands for a carboxylic group e.g. into compounds of the formulae (I), (11), (Ill), (VI), (VII) or (VIII) in which R¹ stands for a —C(═O)N(R³)R⁴ group, can be accomplished by coupling with amines of formula HN(R³)R⁴, in which R³ and R⁴ have the meaning as given for general formula (I) and which are widely commercially available, with a suitable coupling agent, such as HATU, TBTU, PyBOP, PyBrOP or 2,4,6-tripropyl-1,3,5,2,4,6-trioxatriphosphinane 2,4,6-trioxide (also known as T3P).

However, also other routes may be used to synthesize the target compounds (I), in accordance with common general knowledge of a person skilled in the art of organic synthesis. The order of transformations is therefore not intended to be limiting. In addition, interconversion of any of the substituents as defined herein for LG¹, LG², R¹, R^{2a}, R^{2b} and R^{2c} can be achieved before and/or after the exemplified transformations as described supra.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

The IUPAC names of the examples and intermediates were generated using the program ACD/Name batch version 12.01′ from ACD LABS, and were adapted if needed.

The various aspects of the invention described in this application are illustrated by the following examples which are not meant to limit the invention in any way.

### Example 1

### (7S)-4-[(8′-Methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid

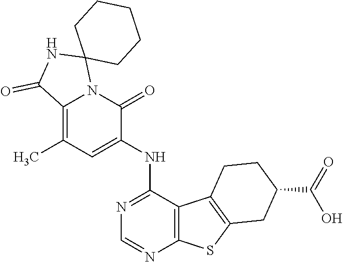 [SMILES: Cc1cc(Nc2ncnc3sc4c(c23)CC[C@H](C(=O)O)C4)c(=O)n2c1C(=O)NC21CCCCC1]

A mixture of 614 mg (1.21 mmol) ethyl (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno [2,3-d]pyrimidine-7-carboxylate (prepared according to example 1a) in 18 mL tetrahydrofuran, 8.5 mL ethanol and 7.3 mL lithium hydroxide solution (1M in water) was stirred at RT overnight. Water was added and the mixture was acidified with hydrochloric acid. After concentration the precipitate was washed with water and dried to give 573 mg (99%) of the title compound.

LC-MS: m/z=480.3 [M+H]⁺.

¹H-NMR (400 MHz, DMSO-d6), δ [ppm]=1.29 (1H), 1.48 (2H), 1.58-1.81 (5H), 2.01 (1H), 2.25 (1H), 2.50 (3H*), 2.89 (1H), 2.93-3.13 (4H), 3.20 (2H), 8.63 (1H), 8.73 (1H), 8.99 (1H), 10.19 (1H), 12.56 (1H);

*: at least partially hidden by solvent peak.

### Example 1a

### Ethyl (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylate

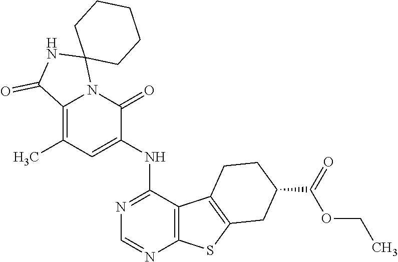 [SMILES: CCOC(=O)[C@H]1CCc2c(sc3ncnc(Nc4cc(C)c5n(c4=O)C4(CCCCC4)NC5=O)c23)C1]

To a solution of 100 mg (361 μmol) ethyl (7S)-4-amino-5,6,7,8-tetrahydro[1]benzothieno[2,3-d]pyrimidine-7-carboxylate (prepared according to example 1b) and 123 mg (397 μmol) 6′-bromo-8′-methyl-2′H-spiro[cyclohexane-1,3′-imidazo[1,5-a]pyridine]-1′,5′-dione (CAS-No: 1849592-70-6; PCT Int. Appl. (2015), WO 2015200481) in 14 mL 1,4-dioxane were added 352 mg cesium carbonate and the mixture was degassed and purged with argon several times. 22.3 mg 4,5-bis(diphenylphosphino)-9,9-dimethylxanthene, 18.4 mg 2-(dicyclohexyl-phosphino)-2′,4′,6′-triisopropylbiphenyl, 8.7 mg palladium(II)acetate and 35.3 mg tris(dibenzylideneacetone)dipalladium(0) were added and the mixture was stirred at 100° C. for 2 hours. Dichloromethane and ethanol were added, the precipitate was filtered off, the filtrate was concentrated and the residue purified by flash chromatography (Biotage SNAP cartridge silica 50 g, ethanol:dichloromethane) to give 130 mg (25%) of the title compound.

LC-MS: m/z=508.3 [M+H]⁺.

### Example 1b

### Ethyl (7S)-4-amino-5,6,7,8-tetrahydro[1]benzothieno[2,3-d]pyrimidine-7-carboxylate

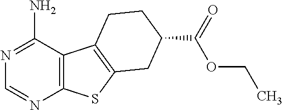 [SMILES: CCOC(=O)[C@H]1CCc2c(sc3ncnc(N)c23)C1]

To a solution of 6.00 g (20.2 mmol) ethyl (7S)-4-chloro-5,6,7,8-tetrahydro[1]benzothieno[2,3-d]pyrimidine-7-carboxylate (prepared according to the procedure described in WO 2015074986) in 156 mL 1,4-dioxane were added 85 mL ammonia solution (33% in water) and the mixture was heated at 120° C. for 1 hour. After concentration the residue was digested with ethanol and dried to give 5.10 g (91%) of the title compound.

LC-MS: m/z=278.2 [M+H]⁺.

### Example 2

### (7S)—N, N-Dimethyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide

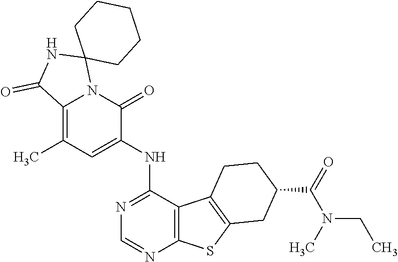 [SMILES: CCN(C)C(=O)[C@H]1CCc2c(sc3ncnc(Nc4cc(C)c5n(c4=O)C4(CCCCC4)NC5=O)c23)C1]

A mixture of 43 mg (90 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.0 mL N,N-dimethylacetamide, 94 μL N-ethyl-N-isopropylpropan-2-amine, 224 μL N-methylmethanamine (2M in tetrahydrofuran) and 160 μL 2,4,6-tripropyl-1,3,5,2,4,6-trioxatriphosphinane 2,4,6-trioxide solution (50% in N,N-dimethylformamide) was stirred at RT overnight. The mixture was concentrated water added, the precipitate was washed with water, ethanol and diethyl ether and dried to give 33.5 mg (70%) of the title compound.

LC-MS: m/z=507.3 [M+H]⁺.

¹H-NMR (400 MHz, DMSO-d6), δ [ppm]=1.27 (1H), 1.48 (2H), 1.58-1.87 (6H), 2.16 (1H), 2.52 (3H*), 2.88 (3H), 2.92-3.05 (4H), 3.11 (3H), 3.16-3.29 (3H), 8.64 (1H), 8.75 (1H), 9.01 (1H), 10.19 (1H);

*: at least partially hidden by solvent peak.

### Example 3

### 6′-{[(7S)-7-(Azetidin-1-ylcarbonyl)-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione

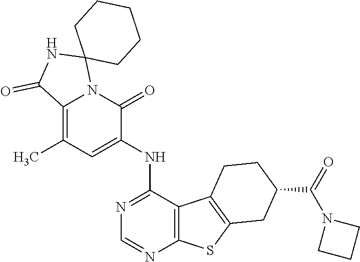 [SMILES: Cc1cc(Nc2ncnc3sc4c(c23)CC[C@H](C(=O)N2CCC2)C4)c(=O)n2c1C(=O)NC21CCCCC1]

A mixture of 43 mg (90 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.0 mL N,N-dimethylacetamide, 125 μL N-ethyl-N-isopropylpropan-2-amine, 94 μL azetidine hydrochloride and 160 μL 2,4,6-tripropyl-1,3,5,2,4,6-trioxatriphosphinane 2,4,6-trioxide solution (50% in N,N-dimethylformamide) was stirred at RT overnight. The mixture was concentrated, water added, the precipitate was washed with water, ethanol and diethyl ether and dried to give 30.6 mg (63%) of the title compound.

LC-MS: m/z=519.3 [M+H]⁺.

¹H-NMR (400 MHz, DMSO-d6), δ [ppm]=1.26 (1H), 1.49 (2H), 1.58-1.88 (6H), 2.14 (1H), 2.22 (2H), 2.52 (3H*), 2.72-2.83 (1H), 2.92 (2H), 2.98 (2H), 3.13-3.27 (2H), 3.89 (2H), 4.26 (2H), 8.64 (1H), 8.75 (1H), 9.01 (1H), 10.19 (1H);

*: at least partially hidden by solvent peak.

### Example 4

### (7S)—N-Methyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide

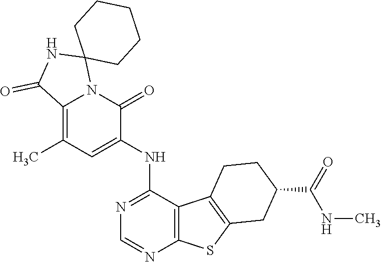 [SMILES: CNC(=O)[C@H]1CCc2c(sc3ncnc(Nc4cc(C)c5n(c4=O)C4(CCCCC4)NC5=O)c23)C1]

A mixture of 55 mg (115 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.8 mL N,N-dimethylacetamide, 120 μL N-ethyl-N-isopropylpropan-2-amine, 287 μL methanamine (2M in tetrahydrofurane) and 205 μL 2,4,6-tripropyl-1,3,5,2,4,6-trioxatriphosphinane 2,4,6-trioxide solution (50% in N,N-dimethylformamide) was stirred at 50° C. overnight. The mixture was concentrated, water added the precipitate was washed with water, ethanol and diethyl ether and dried to give 50 mg (85%) of the title compound.

LC-MS: m/z=493.3 [M+H]⁺.

¹H-NMR (400 MHz, DMSO-d6), δ [ppm]=1.28 (1H), 1.48 (2H), 1.59-1.82 (5H), 1.91 (1H), 2.17 (1H), 2.52 (3H*), 2.63 (3H), 2.69 (1H), 2.91-3.05 (4H), 3.13 (1H), 3.25 (1H), 7.95 (1H), 8.63 (1H), 8.73 (1H), 9.00 (1H), 10.19 (1H);

*: at least partially hidden by solvent peak.

### Example 5

### 1-({(7S)-4-[(8′-Methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile

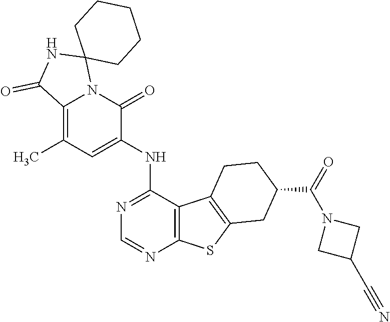 [SMILES: Cc1cc(Nc2ncnc3sc4c(c23)CC[C@H](C(=O)N2CC(C#N)C2)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.5 mL N,N-dimethylacetamide, 163 μL N-ethyl-N-isopropylpropan-2-amine, 62 mg azetidine-3-carbonitrile and 186 μL 2,4,6-tripropyl-1,3,5,2,4,6-trioxatriphosphinane 2,4,6-trioxide solution (50% in N,N-dimethylformamide) was stirred at 50° C. overnight. The mixture was concentrated the precipitate was washed with water, ethanol and diethyl ether and dried to give 53 mg (83%) of the title compound.

LC-MS: m/z=544.3 [M+H]⁺.

¹H-NMR (400 MHz, DMSO-d6), δ [ppm]=1.25 (1H), 1.49 (2H), 1.59-1.88 (5H), 2.18 (1H), 2.52 (3H*), 2.78 (1H), 2.86-3.06 (5H), 3.24 (2H), 3.82 (1H), 4.06 (1H), 4.18 (1H), 4.53 (2H), 8.64 (1H), 8.75 (1H), 9.01 (1H), 10.20 (1H);

*: at least partially hidden by solvent peak.

### Example 6

### 6′-({(7S)-7-[(3-Hydroxy-3-methylazetidin-1-yl)carbonyl]-5,6,7,8-tetrahydro[1]benzothieno[2,3-d]pyrimidin-4-yl}amino)-8′-methyl-2′H-spiro[cyclohexane-1,3′-imidazo[1,5-a]pyridine]-1′,5′-dione

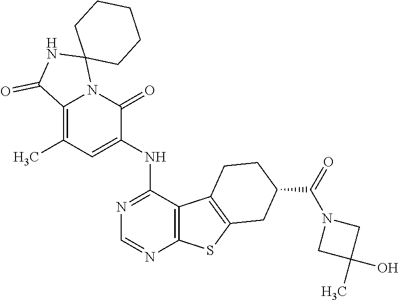 [SMILES: Cc1cc(Nc2ncnc3sc4c(c23)CC[C@H](C(=O)N2CC(C)(O)C2)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.3 mL N,N-dimethylacetamide, 163 μL N-ethyl-N-isopropylpropan-2-amine, 64 mg 3-methylazetidin-3-ol hydrochloride and 186 μL 2,4,6-tripropyl-1,3,5,2,4,6-trioxatriphosphinane 2,4,6-trioxide solution (50% in N,N-dimethylformamide) was stirred at 50° C. overnight. The mixture was concentrated, water added, the precipitate was washed with water, ethanol and diethyl ether and dried to give 50 mg (83%) of the title compound.

LC-MS: m/z=549.3 [M+H]⁺.

¹H-NMR (400 MHz, DMSO-d6), δ [ppm]=1.27 (1H), 1.40 (3H), 1.49 (2H), 1.57-1.90 (6H), 2.15 (1H), 2.52 (3H*), 2.79 (1H), 2.86-3.05 (4H), 3.22 (2H), 3.66-3.81 (2H), 4.09 (2H), 5.66 (1H), 8.63 (1H), 8.74 (1H), 8.99 (1H), 10.19 (1H);

*: at least partially hidden by solvent peak.

### Example 7

### tert-Butyl [1-({(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidin-3-yl]carbamate

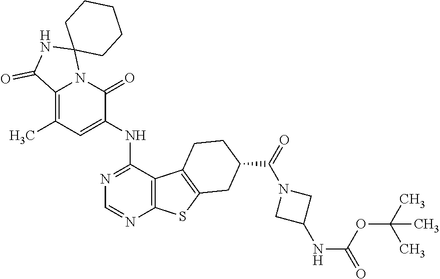 [SMILES: Cc1cc(Nc2ncnc3sc4c(c23)CC[C@H](C(=O)N2CC(NC(=O)OC(C)(C)C)C2)C4)c(=O)n2c1C(=O)NC21CCCCC1]

A mixture of 80 mg (167 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 4.0 mL N,N-dimethylacetamide, 262 μL N-ethyl-N-isopropylpropan-2-amine, 174 mg tert-butyl azetidin-3-ylcarbamate hydrochloride and 298 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 98 mg (88%) of the title compound.

LC-MS: m/z=634.5 [M+H]⁺.

¹H-NMR (400 MHz, DMSO-d6), δ [ppm]=1.27 (2H), 1.39 (9H), 1.48 (2H), 1.57-1.88 (5H), 2.15 (1H), 2.52 (3H*), 2.77 (1H), 2.86-3.08 (4H), 3.14-3.29 (2H), 3.73 (1H), 4.00-4.14 (2H), 4.30 (1H), 4.47 (1H), 7.60 (1H), 8.63 (1H), 8.74 (1H), 9.00 (1H), 10.19 (1H)

*: at least partially hidden by solvent peak.

### Example 8

### 8′-Methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione

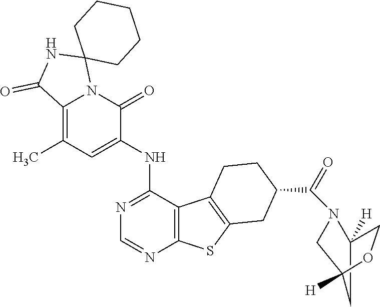 [SMILES: Cc1cc(Nc2ncnc3sc4c(c23)CC[C@H](C(=O)N2C[C@H]3C[C@@H]2CO3)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.5 mL N,N-dimethylacetamide, 163 μL N-ethyl-N-isopropylpropan-2-amine, 71 mg (1R,4R)-2-oxa-5-azabicyclo[2.2.1]heptane hydrochloride and 186 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 53 mg (87%) of the title compound.

LC-MS: m/z=561.4 [M+H]⁺.

¹H-NMR (400 MHz, DMSO-d6), δ [ppm]=1.27 (1H), 1.49 (2H), 1.58-1.96 (8H), 2.19 (1H), 2.52 (3H*), 2.75-3.13 (5H), 3.18-3.30 (3H), 3.50-3.78 (3H), 4.62+4.67 (1H), 4.78+4.90 (1H), 8.63 (1H), 8.73+8.74 (1H), 8.98-9.06 (1H), 10.19 (1H);

*: at least partially hidden by solvent peak.

### Example 9

### 6′-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione

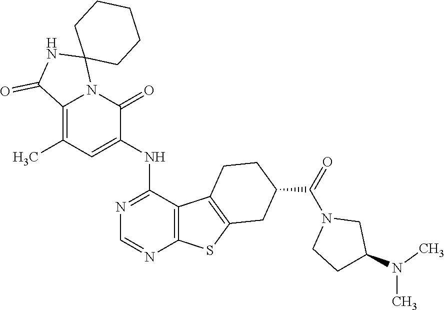 [SMILES: Cc1cc(Nc2ncnc3sc4c(c23)CC[C@H](C(=O)N2CC[C@H](N(C)C)C2)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.5 mL N,N-dimethylacetamide, 109 μL N-ethyl-N-isopropylpropan-2-amine, 60 mg (3S)—N,N-dimethylpyrrolidin-3-amine and 186 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 50 mg (79%) of the title compound.

LC-MS: m/z=576.4 [M+H]⁺.

¹H-NMR (400 MHz, DMSO-d6), δ [ppm]=1.26 (1H), 1.48 (2H), 1.58-1.88 (7H), 1.99-2.24 (2H), 2.17 (6H), 2.52 (3H*), 2.57-2.76 (1H), 2.88-3.07 (5H), 3.16-3.28 (3H), 3.47-3.68 (2H), 3.78+3.87 (1H), 8.63 (1H), 8.73+8.74 (1H), 9.00 (1H), 10.19 (1H);

*: at least partially hidden by solvent peak.

### Example 10

### 8′-Methyl-6′-({(7S)-7-[(4-oxopiperidin-1-yl)carbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione

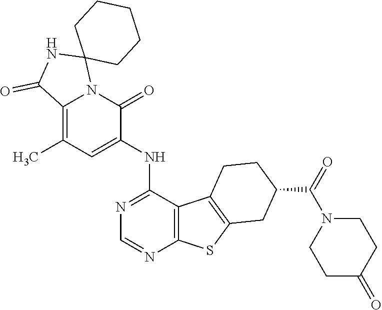 [SMILES: Cc1cc(Nc2ncnc3sc4c(c23)CC[C@H](C(=O)N2CCC(=O)CC2)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.5 mL N,N-dimethylacetamide, 163 μL N-ethyl-N-isopropylpropan-2-amine, 71 mg piperidin-4-one hydrochloride and 186 μL 2,4,6-tripropyl-1,3,5,2,4,6-trioxatriphosphinane 2,4,6-trioxide solution (50% in N,N-dimethylformamide) was stirred at 50° C. for 2 days. Water was added, the precipitate was washed with water, ethanol and diethyl ether and dried to give 39 mg (64%) of the title compound.

LC-MS: m/z=561.3 [M+H]⁺.

¹H-NMR (400 MHz, DMSO-d6), δ [ppm]=1.27 (1H), 1.49 (2H), 1.58-1.81 (5H), 1.87 (1H), 2.22 (1H), 2.39 (2H), 2.44-2.56 (5H*), 2.92-3.12 (4H), 3.22-3.32 (3H*), 3.71 (1H), 3.83-3.98 (3H), 8.64 (1H), 8.75 (1H), 9.01 (1H), 10.19 (1H);

*: at least partially hidden by solvent peak.

### Example 11

### 6′-({(7S)-7-[(3-Aminoazetidin-1-yl)carbonyl]-5,6,7,8-tetrahydro[1]benzothieno[2,3-d]pyrimidin-4-yl}amino)-8′-methyl-2′H-spiro[cyclohexane-1,3′-imidazo[1,5-a]pyridine]-1′,5′-dione

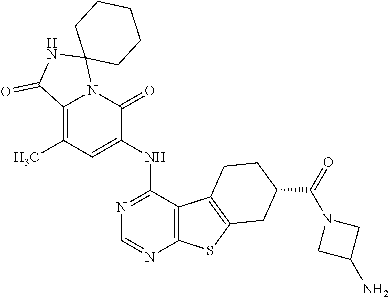 [SMILES: Cc1cc(Nc2ncnc3sc4c(c23)CC[C@H](C(=O)N2CC(N)C2)C4)c(=O)n2c1C(=O)NC21CCCCC1]

A mixture of 91.9 mg (145 μmol) tert-butyl [1-({(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidin-3-yl]carbamate (prepared according to example 7) and 168 μL trifluoroacetic acid in 5.6 mL dichloromethane was stirred at 50° C. overnight. The mixture was poured into aqueous ammonia (25%) and the precipitate washed with water, ethanol and dichloromethane to give 48 mg (59%) of the title compound.

LC-MS: m/z=534.3 [M+H]⁺.

¹H-NMR (400 MHz, DMSO-d6), δ [ppm]=1.25 (1H), 1.49 (2H), 1.59-1.88 (7H), 2.15 (1H), 2.52 (3H*), 2.80 (1H), 2.88-3.51 (5H*), 3.62 (1H), 3.81 (1H), 3.94 (1H), 4.06 (1H), 4.44 (1H), 8.64 (1H), 8.75 (1H), 9.00 (1H), 10.20 (1H);

*: at least partially hidden by solvent peak.

### Example 12

### (7S)—N-[2-(Dimethylamino)ethyl]-N-methyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide

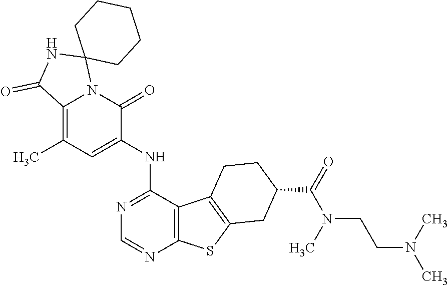 [SMILES: Cc1cc(Nc2ncnc3sc4c(c23)CC[C@H](C(=O)N(C)CCN(C)C)C4)c(=O)n2c1C(=O)NC21CCCCC1]

A mixture of 48 mg (100 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno [2,3-d]pyrimidine-7-carboxylic acid (prepared according to example 1), 3.0 mL N,N-dimethylacetamide, 105 μL N-ethyl-N-isopropylpropan-2-amine, 65 μL N,N,N′-trimethylethane-1,2-diamine and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 51 mg (86%) of the title compound.

LC-MS: m/z=564.4 [M+H]⁺.

¹H-NMR (400 MHz, DMSO-d6), δ [ppm]=1.27 (1H), 1.48 (2H), 1.58-1.92 (6H), 2.14 (1H), 2.16 (3H), 2.18 (3H), 2.35+2.42 (2H), 2.52 (3H*), 2.88+3.11 (3H), 2.91-3.03 (4H), 3.14-3.28 (3H), 3.36-3.62 (2H), 8.63 (1H), 8.74+8.75 (1H), 9.01 (1H), 10.19 (1H);

*: at least partially hidden by solvent peak.

### Example 13

### 8′-Methyl-6′-({(7S)-7-[(3-oxo-8-azabicyclo[3.2.1]oct-8-yl)carbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione

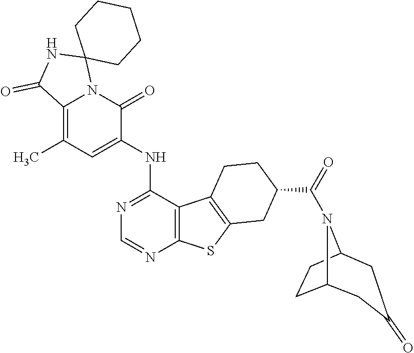 [SMILES: Cc1cc(Nc2ncnc3sc4c(c23)CC[C@H](C(=O)N2C3CCC2CC(=O)C3)C4)c(=O)n2c1C(=O)NC21CCCCC1]

A mixture of 48 mg (100 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 3.0 mL N,N-dimethylacetamide, 157 μL N-ethyl-N-isopropylpropan-2-amine, 81 mg 8-azabicyclo[3.2.1]octan-3-one hydrochloride and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 54 mg (82%) of the title compound.

LC-MS: m/z=587.4 [M+H]⁺.

¹H-NMR (400 MHz, DMSO-d6), δ [ppm]=1.26 (1H), 1.49 (2H), 1.55-1.80 (7H), 1.93 (2H), 2.06-2.36 (4H), 2.52 (3H*), 2.61 (1H), 2.78 (1H), 2.91-3.30 (7H), 4.77 (2H), 8.64 (1H), 8.74+8.75 (1H), 9.02 (1H), 10.20 (1H);

*: at least partially hidden by solvent peak.

### Example 14

### 6′-{[(7S)-7-{[(3S)-3,4-Dimethylpiperazin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione

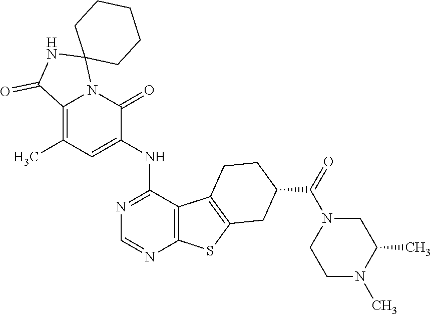 [SMILES: Cc1cc(Nc2ncnc3sc4c(c23)CC[C@H](C(=O)N2CCN(C)[C@@H](C)C2)C4)c(=O)n2c1C(=O)NC21CCCCC1]

A mixture of 45 mg (94 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno [2,3-d]pyrimidine-7-carboxylic acid (prepared according to example 1), 3.0 mL N,N-dimethylacetamide, 245 μL N-ethyl-N-isopropylpropan-2-amine, 88 mg (2S)-1,2-dimethylpiperazine dihydrochloride and 186 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethyl ether and dried to give 47 mg (83%) of the title compound.

LC-MS: m/z=576.4 [M+H]⁺.

¹H-NMR (400 MHz, DMSO-d6), δ [ppm]=0.97-1.06 (3H), 1.29 (1H), 1.48 (2H), 1.58-2.00 (8H), 2.09-2.23 (4H), 2.52 (3H*), 2.71-3.08 (6H), 3.12-3.29 (4H), 3.93 (1H), 4.15+4.26 (1H), 8.64 (1H), 8.75+8.76 (1H), 9.01 (1H), 10.20 (1H);

*: at least partially hidden by solvent peak.

### Example 15

### (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid

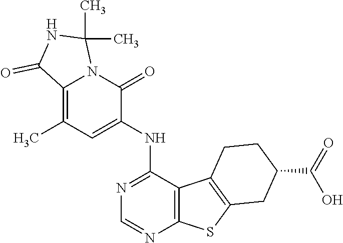 [SMILES: Cc1cc(Nc2ncnc3sc4c(c23)CC[C@H](C(=O)O)C4)c(=O)n2c1C(=O)NC2(C)C]

A mixture of 518 mg (1.11 mmol) ethyl (7S)-4-[(3,3,8-trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylate (prepared according to example 15a) in 16.5 mL tetrahydrofuran, 7.8 mL ethanol and 6.6 mL lithium hydroxide solution (1M in water) was stirred at RT overnight. Water was added and the mixture was acidified with hydrochloric acid. After concentration the precipitate was washed with water and dried to give 468 mg (96%) of the title compound.

LC-MS: m/z=440.3 [M+H]⁺.

¹H-NMR (400 MHz, DMSO-d6), δ [ppm]=1.80 (6H), 2.01 (1H), 2.26 (1H), 2.50 (3H*), 2.89 (1H), 3.01 (1H), 3.10 (1H), 3.322 (2H), 8.64 (1H), 8.73 (1H), 8.98 (1H), 9.63 (1H), 12.51 (1H);

*: at least partially hidden by solvent peak.

### Example 15a

### Ethyl (7S)-4-[(3,3,8-trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylate

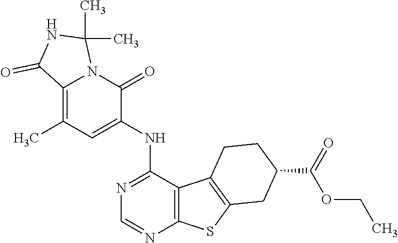 [SMILES: CCOC(=O)[C@H]1CCc2c(sc3ncnc(Nc4cc(C)c5n(c4=O)C(C)(C)NC5=O)c23)C1]

To a solution of 600 mg (2.16 mmol) ethyl (7S)-4-amino-5,6,7,8-tetrahydro[1]benzothieno[2,3-d]pyrimidine-7-carboxylate (prepared according to example 1 b) and 645 mg (2.38 mmol) 6-bromo-3,3,8-trimethyl-2,3-dihydroimidazo[1,5-a]pyridine-1,5-dione (PCT Int. Appl. (2015), WO 2015200481) in 60 mL 1,4-dioxane were added 2.12 g cesium carbonate and the mixture was degassed and purged with argon several times. 134 mg 4,5-bis(diphenylphosphino)-9,9-dimethylxanthene, 110 mg 2-(dicyclohexyl-phosphino)-2′,4′,6′-triisopropylbiphenyl, 52 mg palladium(II)acetate and 212 mg tris(dibenzylideneacetone)-dipalladium(0) were added and the mixture was stirred at 100° C. for 2 hours.

Dichloromethane and ethanol were added, the precipitate was filtered off, the filtrate was concentrated and the residue purified by flash chromatography (Biotage SNAP cartridge silica 100 g, ethanol:dichloromethane) to give 518 mg (51%) of the title compound.

LC-MS: m/z=468.3 [M+H]⁺.

### Example 16

### (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid

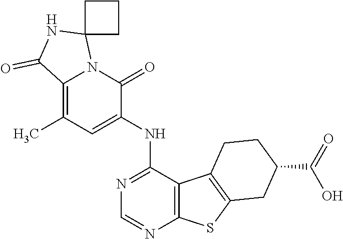 [SMILES: Cc1cc(Nc2ncnc3sc4c(c23)CC[C@H](C(=O)O)C4)c(=O)n2c1C(=O)NC21CCC1]

A mixture of 852 mg (1.78 mmol) ethyl (7S)-4-[(8′-methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylate (prepared according to example 16a) in 26.4 mL tetrahydrofuran, 12.5 mL ethanol and 10.7 mL lithium hydroxide solution (1M in water) was stirred at RT overnight. Water was added and the mixture was acidified with hydrochloric acid. After concentration the precipitate was washed with water and dried to give 800 mg (95%) of the title compound.

LC-MS: m/z=452.3 [M+H]⁺.

¹H-NMR (400 MHz, DMSO-d6), δ [ppm]=1.89-2.08 (2H), 2.13 (1H), 2.23-2.36 (3H), 2.46 (3H), 2.90 (1H), 3.01 (1H), 3.10 (1H), 3.19-3.28 (2H), 3.47 (2H), 8.63 (1H), 8.71 (1H), 8.98 (1H), 10.17 (1H), 12.52 (1H).

### Example 16a

### Ethyl (7S)-4-[(8′-methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylate

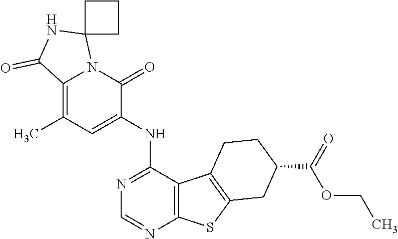 [SMILES: CCOC(=O)[C@H]1CCc2c(sc3ncnc(Nc4cc(C)c5n(c4=O)C4(CCC4)NC5=O)c23)C1]

To a solution of 600 mg (2.16 mmol) ethyl (7S)-4-amino-5,6,7,8-tetrahydro[1]benzothieno[2,3-d]pyrimidine-7-carboxylate (prepared according to example 1 b) and 674 mg (2.38 mmol) 6′-Bromo-8′-methyl-2′H-spiro[cyclobutane-1,3′-imidazo[1,5-a]pyridine]-1′,5′-dione (PCT Int. Appl. (2015), WO 2015200481) in 60 mL 1,4-dioxane were added 2.12 g cesium carbonate and the mixture was degassed and purged with argon several times. 134 mg 4,5-bis(diphenylphosphino)-9,9-dimethylxanthene, 110 mg 2-(dicyclohexyl-phosphino)-2′,4′,6′-triisopropylbiphenyl, 52 mg palladium(II)acetate and 212 mg tris(dibenzylideneacetone)dipalladium(0) were added and the mixture was stirred at 100° C. for 2 hours. Dichloromethane and ethanol were added, the precipitate was filtered off, the filtrate was concentrated and the residue purified by flash chromatography (Biotage SNAP cartridge silica 100 g, ethanol:dichloromethane) to give 852 mg (82%) of the title compound.

LC-MS: m/z=480.3 [M+H]⁺.

### Example 17

### (7S)-4-[(8′-Methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide

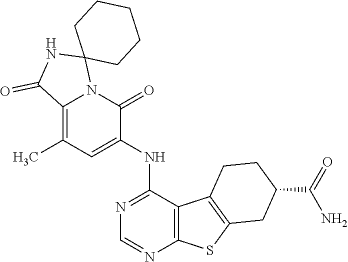 [SMILES: Cc1cc(Nc2ncnc3sc4c(c23)CC[C@H](C(N)=O)C4)c(=O)n2c1C(=O)NC21CCCCC1]

A mixture of 80 mg (167 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 5.0 mL N,N-dimethylacetamide, 262 μL N-ethyl-N-isopropylpropan-2-amine, 5.0 mL ammonia (0.4M in tetrahydrofurane) and 298 μL 2,4,6-tripropyl-1,3,5,2,4,6-trioxatriphosphinane 2,4,6-trioxide solution (50% in N,N-dimethylformamide) was stirred at 60° C. overnight. Water was added, the precipitate was washed with water and ethanol and dried to give 48 mg (57%) of the title compound.

LC-MS: m/z=479.3 [M+H]⁺.

¹H-NMR (400 MHz, DMSO-d6), δ [ppm]=1.28 (1H), 1.48 (2H), 1.58-1.82 (5H), 1.90 (1H), 2.20 (1H), 2.52 (3H*), 2.68 (1H), 2.90-3.18 (5H), 3.25 (1H), 6.99 (1H), 7.47 (1H), 8.63 (1H), 8.72 (1H), 9.00 (1H), 10.19 (1H);

*: at least partially hidden by solvent peak.

### Example 18

### 8′-methyl-6′-{[(7S)-7-{[(3R)-3-methylmorpholin-4-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione

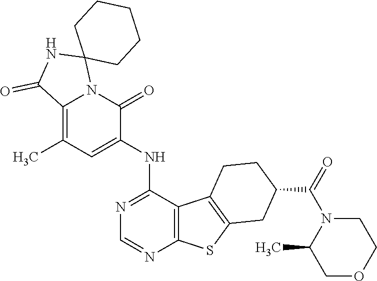 [SMILES: Cc1cc(Nc2ncnc3sc4c(c23)CC[C@H](C(=O)N2CCOC[C@H]2C)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 3.0 mL N,N-dimethylacetamide, 109 μL N-ethyl-N-isopropylpropan-2-amine, 52.7 mg (3R)-3-methylmorpholine and 186 μL 2,4,6-tripropyl-1,3,5,2,4,6-trioxatriphosphinane 2,4,6-trioxide solution (50% in N,N-dimethylformamide) was stirred at RT overnight. Water was added, the precipitate was washed with water and ethanol and dried to give 47 mg (76%) of the title compound.

LC-MS: m/z=563.4 [M+H]⁺.

¹H-NMR (400 MHz, DMSO-d6), δ [ppm]=1.11-1.37 (4H), 1.48 (2H), 1.57-1.81 (5H), 1.88 (1H), 2.12 (1H), 2.52 (3H*), 2.84-3.57 (10H), 3.63 (1H), 3.78+4.12 (1H), 3.85 (1H), 4.18+4.42 (1H), 8.63 (1H), 8.75 (1H), 9.00 (1H), 10.20 (1H);

*: at least partially hidden by solvent peak.

### Example 19

### 6′-{[(7S)-7-{[3-(Dimethylamino)azetidin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione

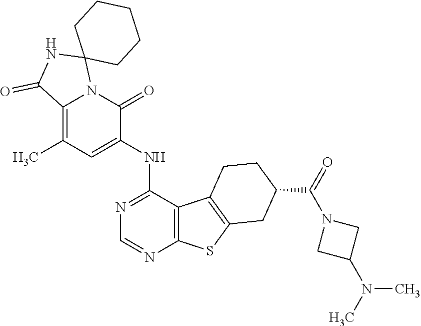 [SMILES: Cc1cc(Nc2ncnc3sc4c(c23)CC[C@H](C(=O)N2CC(N(C)C)C2)C4)c(=O)n2c1C(=O)NC21CCCCC1]

A mixture of 50 mg (104 μmol) (7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 1), 2.5 mL N,N-dimethylacetamide, 272 μL N-ethyl-N-isopropylpropan-2-amine, 90 mg N,N-dimethylazetidin-3-amine dihydrochloride and 186 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water and ethanol and dried to give 37 mg (59%) of the title compound.

LC-MS: m/z=562.4 [M+H]⁺.

¹H-NMR (400 MHz, DMSO-d6), δ [ppm]=1.25 (1H), 1.48 (2H), 1.58-1.88 (5H), 2.09 (3H), 2.10 (3H), 2.13 (1H), 2.52 (3H*), 2.80 (1H), 2.87-3.28 (8H), 3.67 (1H), 3.89 (1H), 4.06 (1H), 4.26 (1H), 8.63 (1H), 8.74 (1H), 9.00 (1H), 10.19 (1H);

*: at least partially hidden by solvent peak.

### Example 20

### (7S)—N,N-Dimethyl-4-[(8′-methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxamide

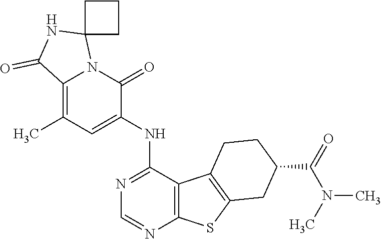 [SMILES: Cc1cc(Nc2ncnc3sc4c(c23)CC[C@H](C(=O)N(C)C)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 116 μL N-ethyl-N-isopropylpropan-2-amine, 224 μL N-methylmethanamine (2M in tetrahydrofuran) and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated, the precipitate was washed with water, ethanol and diethylether and dried to give 31 mg (56%) of the title compound.

LC-MS: m/z=479.3 [M+H]⁺.

¹H-NMR (400 MHz, DMSO-d6), δ [ppm]=1.83 (1H), 1.96 (1H), 2.06-2.22 (2H), 2.26-2.35 (2H), 2.47 (3H), 2.88 (3H), 2.90-3.06 (2H), 3.12 (3H), 3.16-3.30 (3H), 3.46 (2H), 8.64 (1H), 8.75 (1H), 9.00 (1H), 10.17 (1H).

### Example 21

### 6′-{[(7S)-7-(Azetidin-1-ylcarbonyl)-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione

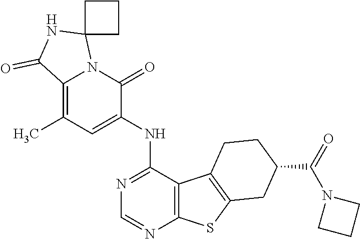 [SMILES: Cc1cc(Nc2ncnc3sc4c(c23)CC[C@H](C(=O)N2CCC2)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 174 μL N-ethyl-N-isopropylpropan-2-amine, 51.8 mg azetidine hydrochloride and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated, the precipitate was washed with water, ethanol and diethylether and dried to give 48 mg (88%) of the title compound.

LC-MS: m/z=491.3 [M+H]⁺.

¹H-NMR (400 MHz, DMSO-d6), δ [ppm]=1.83 (1H), 1.95 (1H), 2.08-2.35 (6H), 2.48 (3H), 2.78 (1H), 2.92 (2H), 3.14-3.54 (4H), 3.89 (2H), 4.27 (2H), 8.63 (1H), 8.73 (1H), 8.98 (1H), 10.17 (1H).

### Example 22

### 1-({(7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile

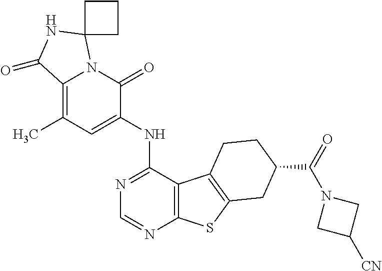 [SMILES: Cc1cc(Nc2ncnc3sc4c(c23)CC[C@H](C(=O)N2CC(C#N)C2)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 174 μL N-ethyl-N-isopropylpropan-2-amine, 65.7 mg azetidine-3-carbonitrile hydrochloride and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated and the residue purified by flash chromatography (Biotage SNAP cartridge 11 g NH+silica 10 g, methanol:dichloromethane) and crystallization from ethanol to give 45 mg (75%) of the title compound.

LC-MS: m/z=516.3 [M+H]⁺.

¹H-NMR (400 MHz, DMSO-d6), δ [ppm]=1.83 (1H), 1.95 (1H), 2.06-2.35 (4H), 2.46 (3H), 2.79 (1H), 2.87-3.03 (2H), 3.17-3.30 (2H), 3.41-3.53 (2H), 3.82 (1H), 4.06 (1H), 4.19 (1H), 4.46-4.61 (2H), 8.64 (1H), 8.73 (1H), 8.99 (1H), 10.17 (1H).

### Example 23

### 6′-{[(7S)-7-{[3-(Dimethylamino)azetidin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione

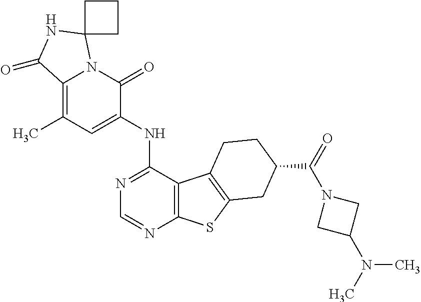 [SMILES: Cc1cc(Nc2ncnc3sc4c(c23)CC[C@H](C(=O)N2CC(N(C)C)C2)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 289 μL N-ethyl-N-isopropylpropan-2-amine, 95.8 mg N,N-dimethylazetidin-3-amine dihydrochloride and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated and the residue purified by flash chromatography (Biotage SNAP cartridge 11 g NH+silica 10 g, methanol:dichloromethane) and crystallization from ethanol to give 22 mg (35%) of the title compound.

LC-MS: m/z=534.4 [M+H]⁺.

¹H-NMR (400 MHz, DMSO-d6), δ [ppm]=1.83 (1H), 1.95 (1H), 2.05-2.21 (2H), 2.10 (3H), 2.12 (3H), 2.25-2.34 (2H), 2.46 (3H), 2.81 (1H), 2.93 (2H), 3.07 (1H), 3.16-3.29 (2H), 3.46 (2H), 3.68 (1H), 3.90 (1H), 4.08 (1H), 4.27 (1H), 8.64 (1H), 8.74 (1H), 8.99 (1H), 10.17 (1H).

### Example 24

### 6′-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione

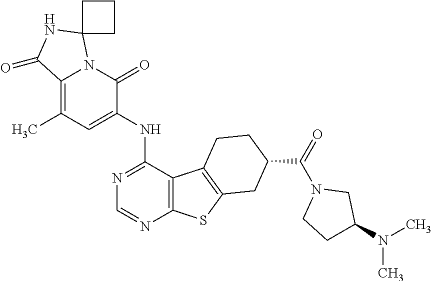 [SMILES: Cc1cc(Nc2ncnc3sc4c(c23)CC[C@H](C(=O)N2CC[C@H](N(C)C)C2)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 116 μL N-ethyl-N-isopropylpropan-2-amine, 70.3 μL (S)-3-(dimethylamino)pyrrolidine and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated and the residue purified by flash chromatography (Biotage SNAP cartridge 11 g NH+silica 10 g, methanol:dichloromethane) and crystallization from ethanol to give 38 mg (60%) of the title compound.

LC-MS: m/z=548.4 [M+H]⁺.

¹H-NMR (400 MHz, DMSO-d6), δ [ppm]=1.58-2.34 (8H), 2.19 (6H), 2.47 (3H), 2.70-3.08 (4H), 3.17-3.68 (7H), 3.80+3.89 (1H), 8.64 (1H), 8.75 (1H), 9.01 (1H), 10.17 (1H).

### Example 25

### 6′-{[(7S)-7-{[(3R)-3,4-Dimethylpiperazin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione

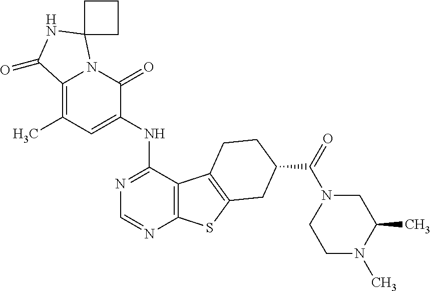 [SMILES: Cc1cc(Nc2ncnc3sc4c(c23)CC[C@H](C(=O)N2CCN(C)[C@H](C)C2)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 289 μL N-ethyl-N-isopropylpropan-2-amine, 104 mg (2R)-1,2-dimethylpiperazine dihydrochloride and 198 μL 2,4,6-tripropyl-1,3,5,2,4,6-trioxatriphosphinane 2,4,6-trioxide solution (50% in N,N-dimethylformamide) was stirred at RT overnight. The mixture was concentrated and the residue purified by flash chromatography (Biotage SNAP cartridge 11 g NH+silica 10 g, methanol:dichloromethane) and crystallization from ethanol to give 42 mg (66%) of the title compound.

LC-MS: m/z=548.4 [M+H]⁺.

¹H-NMR (400 MHz, DMSO-d6), δ [ppm]=1.04 (3H), 1.75-2.36 (10H), 2.47 (3H), 2.69-3.09 (4H), 3.19-3.39 (5H*), 3.46 (2H), 3.94 (1H), 4.17 (1H), 8.64 (1H), 8.75 (1H), 9.00 (1H), 10.18 (1H);

*: at least partially hidden by solvent peak.

### Example 26

### 8′-Methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione

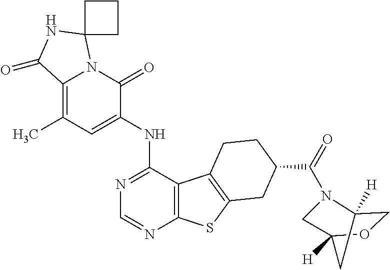 [SMILES: Cc1cc(Nc2ncnc3sc4c(c23)CC[C@H](C(=O)N2C[C@H]3C[C@@H]2CO3)C4)c(=O)n2c1C(=O)NC21CCC1]

A mixture of 50 mg (111 μmol) (7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 16), 3.0 mL N,N-dimethylacetamide, 174 μL N-ethyl-N-isopropylpropan-2-amine, 75.1 mg (1R,4R)-2-oxa-5-azabicyclo[2.2.1]heptane hydrochloride and 198 μL 2,4,6-tripropyl-1,3,5,2,4,6-trioxatriphosphinane 2,4,6-trioxide solution (50% in N,N-dimethylformamide) was stirred at RT overnight. Water was added, the precipitate was washed with water, ethanol and diethylether and dried to give 43 mg (69%) of the title compound.

LC-MS: m/z=533.3 [M+H]⁺.

¹H-NMR (400 MHz, DMSO-d6), δ [ppm]=1.76-2.03 (4H), 2.06-2.36 (4H), 2.47 (3H), 2.81-3.15 (3H), 3.19-3.32 (3H*), 3.46 (2H), 3.57 (1H), 3.63-3.78 (2H), 4.62+4.67 (1H), 4.78+4.92 (1H), 8.64 (1H), 8.73+8.74 (1H), 9.01+9.02 (1H), 10.17 (1H);

*: at least partially hidden by solvent peak.

### Example 27

### (7S)—N,N-Dimethyl-4-[(3,3,8-trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxamide

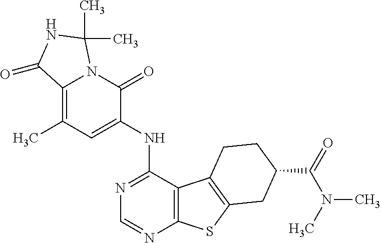 [SMILES: Cc1cc(Nc2ncnc3sc4c(c23)CC[C@H](C(=O)N(C)C)C4)c(=O)n2c1C(=O)NC2(C)C]

A mixture 50 mg (114 mol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 119 μL N-ethyl-N-isopropylpropan-2-amine, 284 μL N-methylmethanamine (2M in tetrahydrofuran) and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 24 mg (43%) of the title compound.

LC-MS: m/z=467.5 [M+H]⁺.

¹H-NMR (400 MHz, DMSO-d6), δ [ppm]=1.80 (7H), 2.16 (1H), 2.52 (3H*), 2.88 (3H), 2.89-3.03 (2H), 3.11 (3H), 3.16-3.29 (3H), 8.64 (1H), 8.74 (1H), 8.99 (1H), 9.63 (1H);

*: at least partially hidden by solvent peak.

### Example 28

### 6-{[(7S)-7-(Azetidin-1-ylcarbonyl)-5,6,7,8-tetrahydro[1]benzothieno[2,3-d]pyrimidin-4-yl]amino}-3,3,8-trimethyl-2,3-dihydroimidazo[1,5-a]pyridine-1,5-dione

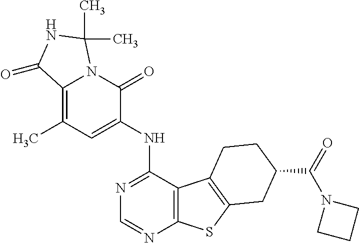 [SMILES: Cc1cc(Nc2ncnc3sc4c(c23)CC[C@H](C(=O)N2CCC2)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 178 μL N-ethyl-N-isopropylpropan-2-amine, 53.2 mg azetidine hydrochloride and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 28 mg (49%) of the title compound.

LC-MS: m/z=479.5 [M+H]⁺.

¹H-NMR (400 MHz, DMSO-d6), δ [ppm]=1.80 (7H), 2.14 (1H), 2.22 (2H), 2.52 (3H*), 2.77 (1H), 2.92 (2H), 3.11-3.29 (2H), 3.89 (2H), 4.26 (2H), 8.64 (1H), 8.74 (1H), 8.98 (1H), 9.63 (1H);

*: at least partially hidden by solvent peak.

### Example 29

### 1-({(7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidin-7-yl}carbonyl)azetidine-3-carbonitrile

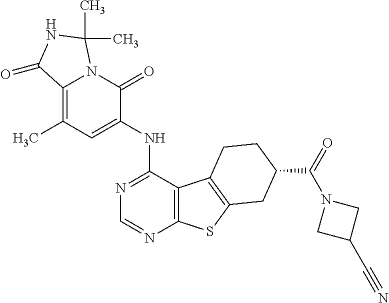 [SMILES: Cc1cc(Nc2ncnc3sc4c(c23)CC[C@H](C(=O)N2CC(C#N)C2)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 178 μL N-ethyl-N-isopropylpropan-2-amine, 67.4 mg azetidine-3-carbonitrile hydrochloride and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 47 mg (74%) of the title compound.

LC-MS: m/z=504.5 [M+H]⁺.

¹H-NMR (400 MHz, DMSO-d6), δ [ppm]=1.80 (7H), 2.18 (1H), 2.52 (3H*), 2.78 (1H), 2.86-3.03 (2H), 3.13-3.28 (2H), 3.82 (1H), 4.05 (1H), 4.19 (1H), 4.54 (2H), 8.64 (1H), 8.74 (1H), 8.99 (1H), 9.63 (1H);

*: at least partially hidden by solvent peak.

### Example 30

### 6-{[(7S)-7-{[3-(Dimethylamino)azetidin-1-yl]carbonyl}-5,6,7,8-tetrahydro[1]benzothieno[2,3-d]pyrimidin-4-yl]amino}-3,3,8-trimethyl-2,3-dihydroimidazo[1,5-a]pyridine-1,5-dione

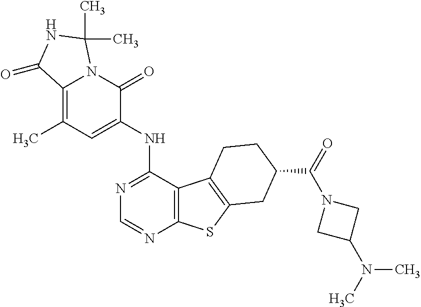 [SMILES: Cc1cc(Nc2ncnc3sc4c(c23)CC[C@H](C(=O)N2CC(N(C)C)C2)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 297 μL N-ethyl-N-isopropylpropan-2-amine, 98.5 mg N,N-dimethylazetidin-3-amine dihydrochloride and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 33 mg (53%) of the title compound.

LC-MS: m/z=522.5 [M+H]⁺.

¹H-NMR (400 MHz, DMSO-d6), δ [ppm]=1.80 (7H), 2.11 (3H), 2.12 (3H), 2.52 (3H*), 2.80 (1H), 2.92 (2H), 3.03-3.33 (4H), 3.68 (1H), 3.89 (1H), 4.07 (1H), 4.27 (1H), 8.63 (1H), 8.73 (1H), 8.98 (1H), 9.63 (1H);

*: at least partially hidden by solvent peak.

### Example 31

### 6-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-3,3,8-trimethyl-2,3-dihydroimidazo[1,5-a]pyridine-1,5-dione

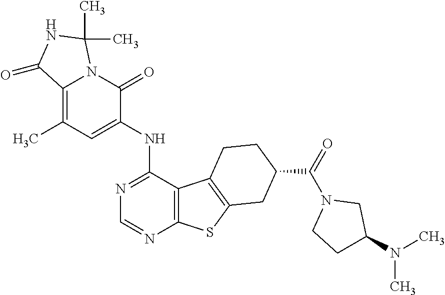 [SMILES: Cc1cc(Nc2ncnc3sc4c(c23)CC[C@H](C(=O)N2CC[C@H](N(C)C)C2)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 119 μL N-ethyl-N-isopropylpropan-2-amine, 72.3 μL (S)-3-(dimethylamino)pyrrolidine and 203 μL 2,4,6-tripropyl-1,3,5,2,4,6-trioxatriphosphinane 2,4,6-trioxide solution (50% in N,N-dimethylformamide) was stirred at RT overnight and at 70° C. for 3 hours. The mixture was concentrated and the residue purified by flash chromatography (Biotage SNAP cartridge silica 10 g, methanol:dichloromethane) and crystallization from ethanol to give 32 mg (50%) of the title compound.

LC-MS: m/z=536.6 [M+H]⁺.

¹H-NMR (400 MHz, DMSO-d6), δ [ppm]=1.57-1.88 (8H), 1.99-2.22 (2H), 2.17 (6H), 2.52 (3H*), 2.61+2.73 (1H), 2.91-3.66 (8H), 3.78+3.86 (1H), 8.64 (1H), 8.74+8.75 (1H), 9.00 (1H), 9.63 (1H);

*: at least partially hidden by solvent peak.

### Example 32

### 6-{[(7S)-7-{[(3R)-3,4-Dimethylpiperazin-1-yl]carbonyl}-5,6,7,8-tetrahydro[1]benzothieno[2,3-d]pyrimidin-4-yl]amino}-3,3,8-trimethyl-2,3-dihydroimidazo[1,5-a]pyridine-1,5-dione

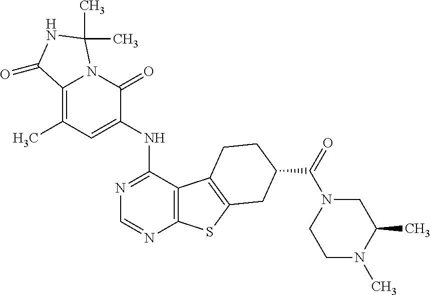 [SMILES: Cc1cc(Nc2ncnc3sc4c(c23)CC[C@H](C(=O)N2CCN(C)[C@H](C)C2)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 297 μL N-ethyl-N-isopropylpropan-2-amine, 106 mg (2R)-1,2-dimethylpiperazine dihydrochloride and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 42 mg (65%) of the title compound.

LC-MS: m/z=536.5 [M+H]⁺.

¹H-NMR (400 MHz, DMSO-d6), δ [ppm]=1.03 (3H), 1.80 (7H), 1.97-2.29 (4H), 2.52 (3H*), 2.69-3.16 (5H), 3.18-3.30 (5H*), 3.93 (1H), 4.17 (1H), 8.64 (1H), 8.74+8.75 (1H), 8.99 (1H), 9.63 (1H);

*: at least partially hidden by solvent peak.

### Example 33

### 3,3,8-Trimethyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2,3-dihydroimidazo[1,5-a]pyridine-1,5-dione

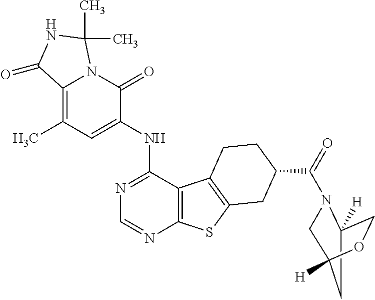 [SMILES: Cc1cc(Nc2ncnc3sc4c(c23)CC[C@H](C(=O)N2C[C@H]3C[C@@H]2CO3)C4)c(=O)n2c1C(=O)NC2(C)C]

A mixture of 50 mg (114 μmol) (7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 15), 3.0 mL N,N-dimethylacetamide, 178 μL N-ethyl-N-isopropylpropan-2-amine, 77.1 mg (1R,4R)-2-oxa-5-azabicyclo[2.2.1]heptane hydrochloride and 203 μL 2,4,6-tripropyl-1,3,5,2,4,6-trioxatriphosphinane 2,4,6-trioxide solution (50% in N,N-dimethylformamide) was stirred at RT for 2.5 days. Water was added, the precipitate was washed with water, ethanol and diethylether and dried to give 45 mg (68%) of the title compound.

LC-MS: m/z=521.5 [M+H]⁺.

¹H-NMR (400 MHz, DMSO-d6), δ [ppm]=1.74-1.96 (3H), 1.80+1.81 (6H), 2.15+2.22 (1H), 2.52 (3H*), 2.77-3.41 (6H), 3.56 (1H), 3.62-3.78 (2H), 4.61+4.66 (1H), 4.78+4.90 (1H), 8.64 (1H), 8.73+8.74 (1H), 9.00+9.01 (1H), 9.63 (1H);

*: at least partially hidden by solvent peak.

### Example 34

### (7S)-4-[(8′-Methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid

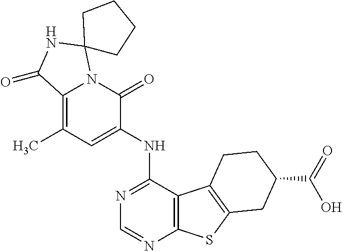 [SMILES: Cc1cc(Nc2ncnc3sc4c(c23)CC[C@H](C(=O)O)C4)c(=O)n2c1C(=O)NC21CCCC1]

A mixture of 841 mg (1.70 mmol) ethyl (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylate (prepared according to example 34a) in 30 mL tetrahydrofuran, 13 mL ethanol and 10 mL lithium hydroxide solution (1M in water) was stirred at RT overnight. Water was added and the mixture was acidified with hydrochloric acid. After concentration the precipitate was washed with water and dried to give 760 mg (91%) of the title compound.

LC-MS: m/z=466.5 [M+H]⁺.

¹H-NMR (400 MHz, DMSO-d6), δ [ppm]=1.69 (2H), 1.78-2.07 (5H), 2.25 (1H), 2.52 (3H*), 2.80-2.93 (3H), 2.97-3.13 (2H), 3.21 (2H), 8.63 (1H), 8.74 (1H), 8.96 (1H), 9.99 (1H), 12.51 (1H);

*: at least partially hidden by solvent peak.

### Example 34a

### Ethyl (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylate

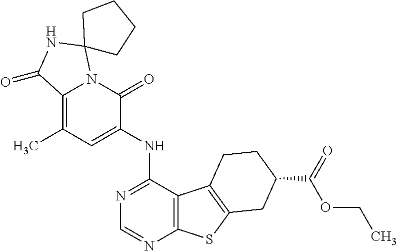 [SMILES: CCOC(=O)[C@H]1CCc2c(sc3ncnc(Nc4cc(C)c5n(c4=O)C4(CCCC4)NC5=O)c23)C1]

To a solution of 600 mg (2.16 mmol) ethyl (7S)-4-amino-5,6,7,8-tetrahydro[1]benzothieno[2,3-d]pyrimidine-7-carboxylate (prepared according to example 1 b) and 707 mg (2.38 mmol) 6′-bromo-8′-methyl-2′H-spiro[cyclopentane-1,3′-imidazo[1,5-a]pyridine]-1′,5′-dione (CAS-No: 1849592-55-7; PCT Int. Appl. (2015), WO 2015200481) in 85 mL 1,4-dioxane were added 2.12 g cesium carbonate and the mixture was degassed and purged with argon several times. 134 mg 4,5-bis(diphenylphosphino)-9,9-dimethylxanthene, 110 mg 2-(dicyclohexyl-phosphino)-2′,4′,6′-triisopropylbiphenyl, 52 mg palladium(II)acetate and 212 mg tris(dibenzylideneacetone)dipalladium(0) were added and the mixture was stirred at 100° C. for 2 hours. Dichloromethane and ethanol were added, the precipitate was filtered off, the filtrate was concentrated and the residue purified by crystallization from methanol to give 843 mg (79%) of the title compound.

LC-MS: m/z=494.5 [M+H]⁺.

### Example 35

### 6′-{[(7S)-7-{[(3R)-3,4-Dimethylpiperazin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione

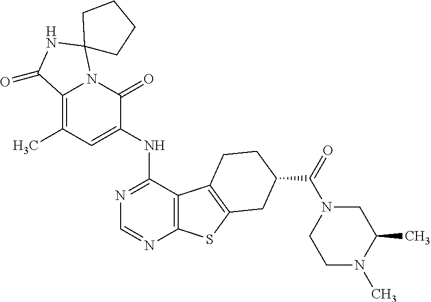 [SMILES: Cc1cc(Nc2ncnc3sc4c(c23)CC[C@H](C(=O)N2CCN(C)[C@H](C)C2)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 281 μL N-ethyl-N-isopropylpropan-2-amine, 100 mg (2R)-1,2-dimethylpiperazine dihydrochloride and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 38 mg (60%) of the title compound.

LC-MS: m/z=562.6 [M+H]⁺.

¹H-NMR (400 MHz, DMSO-d6), δ [ppm]=1.01 (3H), 1.70 (2H), 1.78-2.16 (8H), 2.19 (3H), 2.52 (3H*), 2.69-2.95 (5H), 3.00 (1H), 3.16-3.30 (4H), 3.91 (1H), 4.16 (1H), 8.63 (1H), 8.75 (1H), 8.96 (1H), 10.00 (1H);

*: at least partially hidden by solvent peak.

### Example 36

### 8′-Methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione

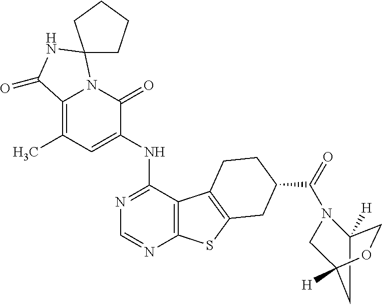 [SMILES: Cc1cc(Nc2ncnc3sc4c(c23)CC[C@H](C(=O)N2C[C@H]3C[C@@H]2CO3)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 168 μL N-ethyl-N-isopropylpropan-2-amine, 72.8 mg (1R,4R)-2-oxa-5-azabicyclo[2.2.1]heptane hydrochloride and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 42 mg (68%) of the title compound.

LC-MS: m/z=547.6 [M+H]⁺.

¹H-NMR (400 MHz, DMSO-d6), δ [ppm]=1.70 (2H), 1.76-2.05 (7H), 2.15+2.22 (1H), 2.52 (3H*), 2.79-3.14 (5H), 3.17-3.38 (3H), 3.56 (1H), 3.63-3.79 (2H), 4.61+4.66 (1H), 4.78+4.91 (1H), 8.64 (1H), 8.74+8.76 (1H), 8.99 (1H), 10.00 (1H);

*: at least partially hidden by solvent peak.

### Example 37

### (7S)—N,N-Dimethyl-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxamide

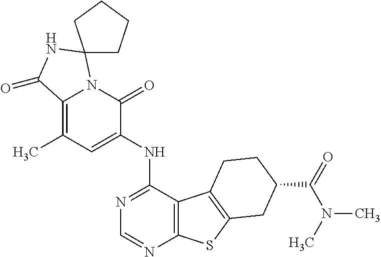 [SMILES: Cc1cc(Nc2ncnc3sc4c(c23)CC[C@H](C(=O)N(C)C)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 112 μL N-ethyl-N-isopropylpropan-2-amine, 269 μL N-methylmethanamine (2M in tetrahydrofuran) and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 36 mg (64%) of the title compound.

LC-MS: m/z=493.5 [M+H]⁺.

¹H-NMR (400 MHz, DMSO-d6), δ [ppm]=1.70 (2H), 1.76-1.90 (3H), 1.97 (2H), 2.15 (1H), 2.52 (3H*), 2.80-3.05 (4H), 2.88 (3H), 3.11 (3H), 3.16-3.29 (3H), 8.63 (1H), 8.75 (1H), 8.96 (1H), 10.00 (1H);

*: at least partially hidden by solvent peak.

### Example 38

### 6′-{[(7S)-7-(Azetidin-1-ylcarbonyl)-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione

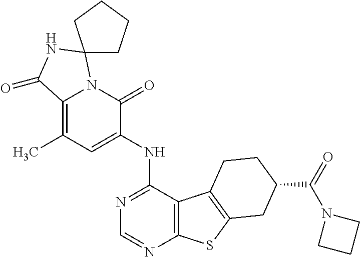 [SMILES: Cc1cc(Nc2ncnc3sc4c(c23)CC[C@H](C(=O)N2CCC2)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.7 mL N,N-dimethylacetamide, 150 μL N-ethyl-N-isopropylpropan-2-amine, 40.2 mg azetidine hydrochloride and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 43 mg (75%) of the title compound.

LC-MS: m/z=505.6 [M+H]⁺.

¹H-NMR (400 MHz, DMSO-d6), δ [ppm]=1.70 (2H), 1.76-1.92 (3H), 1.97 (2H), 2.13 (1H), 2.22 (2H), 2.52 (3H*), 2.72-2.96 (5H), 3.11-3.29 (2H), 3.89 (2H), 4.26 (2H), 8.63 (1H), 8.75 (1H), 8.96 (1H), 10.00 (1H);

*: at least partially hidden by solvent peak.

### Example 39

### 1-({(7S)-4-[(8′-Methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile

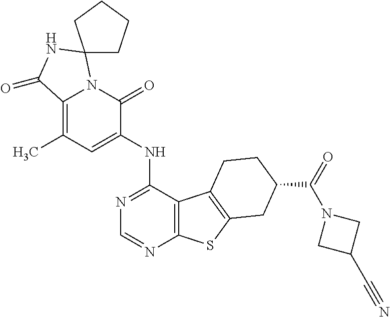 [SMILES: Cc1cc(Nc2ncnc3sc4c(c23)CC[C@H](C(=O)N2CC(C#N)C2)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 168 μL N-ethyl-N-isopropylpropan-2-amine, 63.7 mg azetidine-3-carbonitrile hydrochloride and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 44 mg (73%) of the title compound.

LC-MS: m/z=530.5 [M+H]⁺.

¹H-NMR (400 MHz, DMSO-d6), δ [ppm]=1.70 (2H), 1.76-1.92 (3H), 1.98 (2H), 2.10-2.23 (1H), 2.52 (3H*), 2.73-3.03 (5H), 3.13-3.29 (2H), 3.82 (1H), 4.05 (1H), 4.19 (1H), 4.47-4.60 (2H), 8.64 (1H), 8.75 (1H), 8.97 (1H), 10.00 (1H);

*: at least partially hidden by solvent peak.

### Example 40

### 6′-{[(7S)-7-{[3-(Dimethylamino)azetidin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione

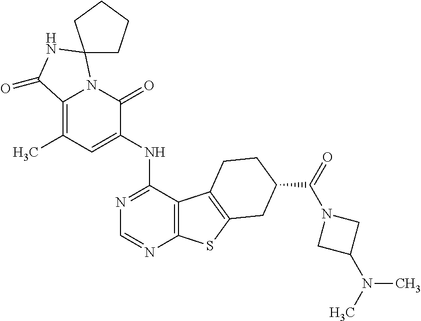 [SMILES: Cc1cc(Nc2ncnc3sc4c(c23)CC[C@H](C(=O)N2CC(N(C)C)C2)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 281 μL N-ethyl-N-isopropylpropan-2-amine, 93.0 mg N,N-dimethylazetidin-3-amine dihydrochloride and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 19 mg (30%) of the title compound.

LC-MS: m/z=548.6 [M+H]⁺.

¹H-NMR (400 MHz, DMSO-d6), δ [ppm]=1.69 (2H), 1.76-1.90 (3H), 1.97 (2H), 2.08 (3H), 2.10 (3H), 2.13 (1H), 2.52 (3H*), 2.76-2.89 (3H), 2.89-2.97 (2H), 3.05 (1H), 3.12-3.30 (2H), 3.67 (1H), 3.89 (1H), 4.06 (1H), 4.26 (1H), 8.64 (1H), 8.76 (1H), 8.97 (1H), 10.00 (1H);

*: at least partially hidden by solvent peak.

### Example 41

### 6′-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione

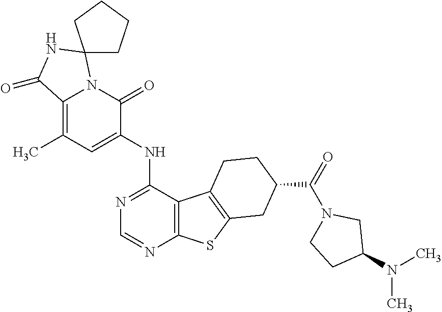 [SMILES: Cc1cc(Nc2ncnc3sc4c(c23)CC[C@H](C(=O)N2CC[C@H](N(C)C)C2)C4)c(=O)n2c1C(=O)NC21CCCC1]

A mixture of 50 mg (107 μmol) (7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 34), 4.0 mL N,N-dimethylacetamide, 112 μL N-ethyl-N-isopropylpropan-2-amine, 68.2 μL (S)-3-(dimethylamino)pyrrolidine and 192 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 31 mg (49%) of the title compound.

LC-MS: m/z=562.6 [M+H]⁺.

¹H-NMR (400 MHz, DMSO-d6), δ [ppm]=1.57-1.90 (6H), 1.92-2.22 (4H), 2.17 (6H), 2.52 (3H*), 2.61+2.73 (1H), 2.79-3.05 (5H), 3.17-3.30 (3H), 3.39-3.68 (2H), 3.78+3.87 (1H), 8.63 (1H), 8.75+8.76 (1H), 8.97 (1H), 10.00 (1H);

*: at least partially hidden by solvent peak.

### Example 42

### (7S)-4-[(4,4-Difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid

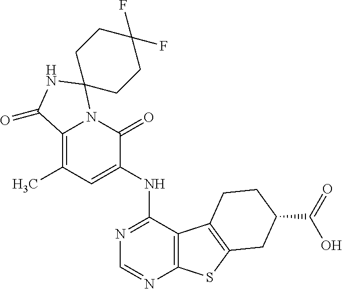 [SMILES: Cc1cc(Nc2ncnc3sc4c(c23)CC[C@H](C(=O)O)C4)c(=O)n2c1C(=O)NC21CCC(F)(F)CC1]

A mixture of 1.17 g (2.15 mmol) ethyl (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylate (prepared according to example 42a) in 35 mL tetrahydrofuran, 15 mL ethanol and 12.9 mL lithium hydroxide solution (1M in water) was stirred at RT overnight. Water was added and the mixture was acidified with hydrochloric acid. After concentration the precipitate was washed with water and dried to give 867 mg (74%) of the title compound.

LC-MS: m/z=516.5 [M+H]⁺.

¹H-NMR (400 MHz, DMSO-d6), δ [ppm]=1.68 (2H), 2.00 (1H), 2.14-2.35 (5H), 2.52 (3H*), 2.87 (1H), 3.00 (1H), 3.10 (1H), 3.15-3.30 (4H), 8.63 (1H), 8.74 (1H), 8.96 (1H), 10.39 (1H), 12.53 (1H);

*: at least partially hidden by solvent peak.

### Example 42a

### Ethyl (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylate

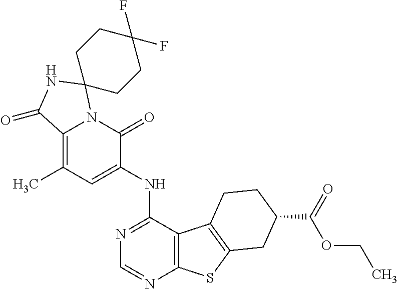 [SMILES: CCOC(=O)[C@H]1CCc2c(sc3ncnc(Nc4cc(C)c5n(c4=O)C4(CCC(F)(F)CC4)NC5=O)c23)C1]

To a solution of 600 mg (2.16 mmol) ethyl (7S)-4-amino-5,6,7,8-tetrahydro[1]benzothieno[2,3-d]pyrimidine-7-carboxylate (prepared according to example 1 b) and 826 mg (2.38 mmol) 6′-bromo-4,4-difluoro-8′-methyl-2′H-spiro[cyclohexane-1,3′-imidazo[1,5-a]pyridine]-1′,5′-dione (PCT Int. Appl. (2015), WO 2015200481) in 60 mL 1,4-dioxane were added 2.12 g cesium carbonate and the mixture was degassed and purged with argon several times. 134 mg 4,5-bis(diphenylphosphino)-9,9-dimethylxanthene, 110 mg 2-(dicyclohexyl-phosphino)-2′,4′,6′-triisopropylbiphenyl, 52 mg palladium(II)acetate and 212 mg tris(dibenzylideneacetone)dipalladium(0) were added and the mixture was stirred at 100° C. for 2 hours. Dichloromethane and ethanol were added, the precipitate was filtered off, the filtrate was concentrated and the residue purified by flash chromatography (Biotage SNAP cartridge silica 100 g, ethanol:dichloromethane) and crystallization from ethanol to give 1.17 g (99%) of the title compound.

LC-MS: m/z=544.4 [M+H]⁺.

### Example 43

### 8′-Methyl-6′-(5,6,7,8-tetrahydro[1]benzothieno[2,3-d]pyrimidin-4-ylamino)-2′H-spiro[cyclohexane-1,3′-imidazo[1,5-a]pyridine]-1′,5′-dione

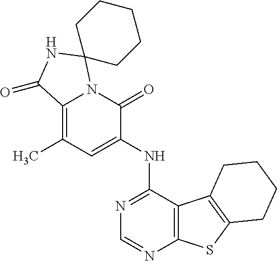 [SMILES: Cc1cc(Nc2ncnc3sc4c(c23)CCCC4)c(=O)n2c1C(=O)NC21CCCCC1]

To a solution of 100 mg (321 μmol) 6′-bromo-8′-methyl-2′H-spiro[cyclohexane-1,3′-imidazo[1,5-a]pyridine]-1′,5′-dione (CAS-No: 1849592-70-6; PCT Int. Appl. (2015), WO 2015200481) and 72.6 mg (353 μmol) 5,6,7,8-tetrahydro[1]benzothieno[2,3-d]pyrimidin-4-amine (CAS-No: 4994-88-1) in 12 mL 1,4-dioxane was added 314 mg cesium carbonate and the mixture was degassed and purged with argon several times. 19.9 mg 4,5-bis(diphenylphosphino)-9,9-dimethylxanthene, 16.4 mg 2-(dicyclohexyl-phosphino)-2′,4′,6′-triisopropylbiphenyl, 7.7 mg palladium(II)acetate and 31.5 mg tris(dibenzylideneacetone) dipalladium(0) were added and the mixture was stirred at 100° C. for 2 hours. The mixture was concentrated and the residue purified by flash chromatography (Biotage SNAP cartridge silica 25 g, methanol:dichloromethane) to give 125 mg (85%) of the title compound.

LC-MS: m/z=436.4 [M+H]⁺.

¹H-NMR (400 MHz, DMSO-d6), δ [ppm]=1.27 (1H), 1.48 (2H), 1.57-1.81 (5H), 1.87 (2H), 1.95 (2H), 2.52 (3H*), 2.85 (2H), 2.98 (2H), 3.15 (2H), 8.63 (1H), 8.74 (1H), 9.04 (1H), 10.19 (1H);

*: at least partially hidden by solvent peak.

### Example 44

### (7S)-4-[(8-Methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid

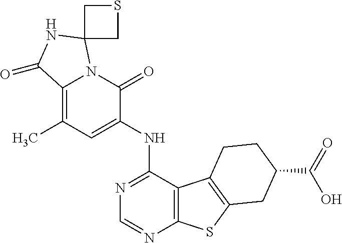 [SMILES: Cc1cc(Nc2ncnc3sc4c(c23)CC[C@H](C(=O)O)C4)c(=O)n2c1C(=O)NC21CSC1]

A mixture of 380 mg (764 μmol) ethyl (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate (prepared according to example 44a) in 11.4 mL tetrahydrofuran, 5.4 mL ethanol and 4.6 mL lithium hydroxide solution (1M in water) was stirred at RT overnight. Water was added and the mixture was acidified with hydrochloric acid. After concentration the precipitate was washed with water and dried to give 350 mg (93%) of the title compound.

LC-MS: m/z=470.5 [M+H]⁺.

¹H-NMR (400 MHz, DMSO-d6), δ [ppm]=2.04 (1H), 2.30 (2H), 2.45 (3H), 2.91 (1H), 3.02 (1H), 3.11 (1H), 3.22-3.30 (3H), 4.69 (2H), 8.65 (1H), 8.72 (1H), 8.98 (1H), 10.72 (1H), 12.52 (1H).

### Example 44a

### Ethyl (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate

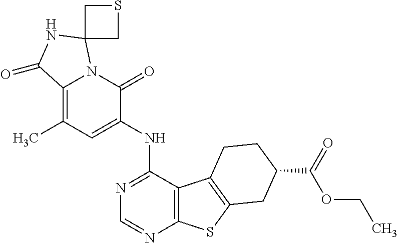 [SMILES: CCOC(=O)[C@H]1CCc2c(sc3ncnc(Nc4cc(C)c5n(c4=O)C4(CSC4)NC5=O)c23)C1]

To a solution of 468 mg (1.69 mmol) ethyl (7S)-4-amino-5,6,7,8-tetrahydro[1]benzothieno[2,3-d]pyrimidine-7-carboxylate (prepared according to example 1 b) and 560 mg (1.86 mmol) 6-bromo-8-methyl-2H-spiro[imidazo[1,5-a]pyridine-3,3′-thietane]-1,5-dione (prepared according to example 44b) in 46.9 mL 1,4-dioxane were added 1.65 g cesium carbonate and the mixture was degassed and purged with argon several times. 105 mg 4,5-bis(diphenylphosphino)-9,9-dimethylxanthene, 86.2 mg 2-(dicyclohexyl-phosphino)-2′,4′,6′-triisopropylbiphenyl, 40.6 mg palladium(II)acetate and 166 mg tris(dibenzylideneacetone)dipalladium(0) were added and the mixture was stirred at 100° C. for 2 hours. Dichloromethane and ethanol were added, the precipitate was filtered off, the filtrate was concentrated and the residue purified by flash chromatography (Biotage SNAP cartridge silica 100 g, ethanol:dichloromethane) and crystallization from ethanol to give 380 mg (45%) of the title compound.

LC-MS: m/z=498.5 [M+H]⁺.

### Example 44b

### 6-Bromo-8-methyl-2H-spiro[imidazo[1,5-a]pyridine-3,3′-thietane]-1,5-dione

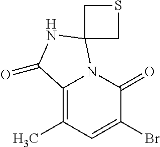 [SMILES: Cc1cc(Br)c(=O)n2c1C(=O)NC21CSC1]

To a solution of 500 mg (2.16 mmol) 5-bromo-3-methyl-6-oxo-1,6-dihydropyridine-2-carbaldehyde (prepared according to PCT Int. Appl. (2015), WO 2015200481) and 286 mg (3.25 mmol) thietan-3-one in 6 mL 1,4-dioxane were added 57 μl sulfuric acid and the mixture was stirred at 95° C. for 3 hours. After concentration water was added, the precipitate washed with water and diethyl ether and dried to give 560 mg (86%) of the title compound.

LC-MS: m/z=301.3 [M+H]⁺.

### Example 45

### (7S)—N,N-Dimethyl-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxamide

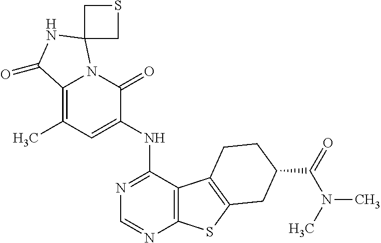 [SMILES: Cc1cc(Nc2ncnc3sc4c(c23)CC[C@H](C(=O)N(C)C)C4)c(=O)n2c1C(=O)NC21CSC1]

A mixture of 52 mg (111 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.1 mL N,N-dimethylacetamide, 116 μL N-ethyl-N-isopropylpropan-2-amine, 277 μL N-methylmethanamine (2M in tetrahydrofuran) and 198 μL 2,4,6-tripropyl-1,3,5,2,4,6-trioxatriphosphinane 2,4,6-trioxide solution (50% in N,N-dimethylformamide) was stirred at RT overnight. 111 μL N-methylmethanamine (2M in tetrahydrofuran) were added and stirring continued at 50° C. for 3 hours. Water was added, the precipitate was washed with water, ethanol and diethylether and dried to give 22 mg (40%) of the title compound.

LC-MS: m/z=497.5 [M+H]⁺.

¹H-NMR (400 MHz, DMSO-d6), δ [ppm]=1.84 (1H), 2.21 (1H), 2.46 (3H), 2.88 (3H), 2.93 (1H), 3.02 (1H), 3.13 (3H), 3.21 (1H), 3.28 (2H), 3.31 (2H), 4.68 (2H), 8.65 (1H), 8.74 (1H), 8.99 (1H), 10.72 (1H).

### Example 46

### 6-{[(7S)-7-(Azetidin-1-ylcarbonyl)-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione

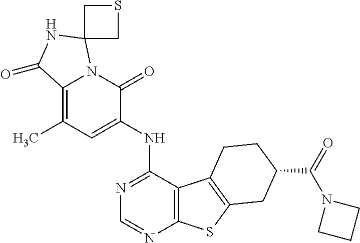 [SMILES: Cc1cc(Nc2ncnc3sc4c(c23)CC[C@H](C(=O)N2CCC2)C4)c(=O)n2c1C(=O)NC21CSC1]

A mixture of 50 mg (106 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.0 mL N,N-dimethylacetamide, 167 μL N-ethyl-N-isopropylpropan-2-amine, 49.8 mg azetidine hydrochloride and 190 μL 2,4,6-tripropyl-1,3,5,2,4,6-trioxatriphosphinane 2,4,6-trioxide solution (50% in N,N-dimethylformamide) was stirred at RT overnight. Another 49.8 mg azetidine hydrochloride were added and stirring continued at 70° C. for 1.5 days. After concentration the residue was purified by flash chromatography (Biotage SNAP cartridge 11 g NH+silica 10 g, methanol:dichloromethane) to give 11 mg (19%) of the title compound.

LC-MS: m/z=509.5 [M+H]⁺.

¹H-NMR (400 MHz, DMSO-d6), δ [ppm]=1.84 (1H), 2.15-2.26 (3H), 2.46 (3H), 2.79 (1H), 2.94 (2H), 3.28 (2H), 3.31 (2H), 3.90 (2H), 4.28 (2H), 4.68 (2H), 8.65 (1H), 8.74 (1H), 9.00 (1H), 10.72 (1H).

### Example 47

### 1-({(7S)-4-[(8-Methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidin-7-yl}carbonyl)azetidine-3-carbonitrile

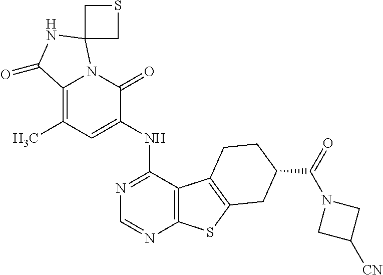 [SMILES: Cc1cc(Nc2ncnc3sc4c(c23)CC[C@H](C(=O)N2CC(C#N)C2)C4)c(=O)n2c1C(=O)NC21CSC1]

A mixture of 52 mg (111 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.1 mL N,N-dimethylacetamide, 174 μL N-ethyl-N-isopropylpropan-2-amine, 65.7 mg azetidine-3-carbonitrile hydrochloride and 198 μL 2,4,6-tripropyl-1,3,5,2,4,6-trioxatriphosphinane 2,4,6-trioxide solution (50% in N,N-dimethylformamide) was stirred at RT overnight. Another 26.3 mg azetidine-3-carbonitrile hydrochloride were added and stirring continued at 50° C. for 3 hours. Water was added, the precipitate was washed with water, ethanol and diethylether and crystallized from ethanol/DMSO to give 36 mg (61%) of the title compound.

LC-MS: m/z=534.5 [M+H]⁺.

¹H-NMR (400 MHz, DMSO-d6), Shift [ppm]=1.85 (1H), 2.22 (1H), 2.46 (3H), 2.81 (1H), 2.87-3.04 (2H), 3.28 (2H), 3.31 (2H), 3.82 (1H), 4.06 (1H), 4.20 (1H), 4.48-4.63 (2H), 4.68 (2H), 8.65 (1H), 8.73 (1H), 9.00 (1H), 10.72 (1H).

### Example 48

### 6-{[(7S)-7-{[3-(Dimethylamino)azetidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione

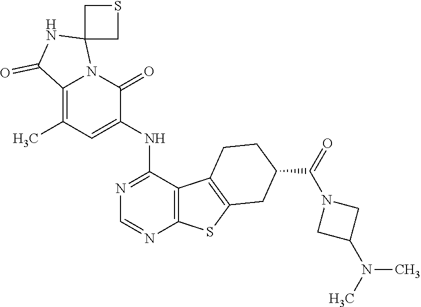 [SMILES: Cc1cc(Nc2ncnc3sc4c(c23)CC[C@H](C(=O)N2CC(N(C)C)C2)C4)c(=O)n2c1C(=O)NC21CSC1]

A mixture of 50 mg (106 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.0 mL N,N-dimethylacetamide, 278 μL N-ethyl-N-isopropylpropan-2-amine, 92.2 mg N,N-dimethylazetidin-3-amine dihydrochloride and 173 μL 2,4,6-tripropyl-1,3,5,2,4,6-trioxatriphosphinane 2,4,6-trioxide solution (50% in N,N-dimethylformamide) was stirred at RT overnight. Another 18.4 mg N,N-dimethylazetidin-3-amine dihydrochloride were added and stirring continued at 50° C. for 3 hours. Water was added, the precipitate was washed with water, ethanol and diethylether and purified by flash chromatography (Biotage SNAP cartridge 11 g NH+silica 10 g, methanol:dichloromethane) to give 20 mg (34%)

LC-MS: m/z=552.5 [M+H]⁺.

¹H-NMR (400 MHz, DMSO-d6), δ [ppm]=1.84 (1H), 2.09 (3H), 2.10 (3H), 2.19 (1H), 2.46 (3H), 2.82 (1H), 2.94 (2H), 3.06 (1H), 3.28 (2H), 3.31 (2H), 3.67 (1H), 3.89 (1H), 4.05+4.11 (1H), 4.25+4.30 (1H), 4.68 (2H), 8.65 (1H), 8.75 (1H), 9.00 (1H), 10.72 (1H).

### Example 49

### 6-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione

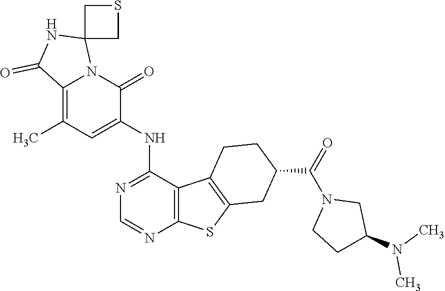 [SMILES: Cc1cc(Nc2ncnc3sc4c(c23)CC[C@H](C(=O)N2CC[C@H](N(C)C)C2)C4)c(=O)n2c1C(=O)NC21CSC1]

A mixture of 50 mg (106 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.0 mL N,N-dimethylacetamide, 111 μL N-ethyl-N-isopropylpropan-2-amine, 67.6 μL (S)-3-(dimethylamino)pyrrolidine and 190 μL 2,4,6-tripropyl-1,3,5,2,4,6-trioxatriphosphinane 2,4,6-trioxide solution (50% in N,N-dimethylformamide) was stirred at RT overnight. After concentration the residue was purified by flash chromatography (Biotage SNAP cartridge NH 11 g, methanol:dichloromethane) to give 21 mg (33%) of the title compound.

LC-MS: m/z=566.6 [M+H]⁺.

¹H-NMR (400 MHz, DMSO-d6), δ [ppm]=1.58-1.93 (2H), 1.98-2.29 (2H), 2.18 (6H), 2.46 (3H), 2.61+2.75 (1H), 2.91-3.68 (6H), 3.28 (2H), 3.30 (2H), 3.81+3.90 (1H), 4.68 (2H), 8.65 (1H), 8.75 (1H), 9.01 (1H), 10.72 (1H).

### Example 50

### 6-{[(7S)-7-{[(3R)-3,4-dimethylpiperazin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione

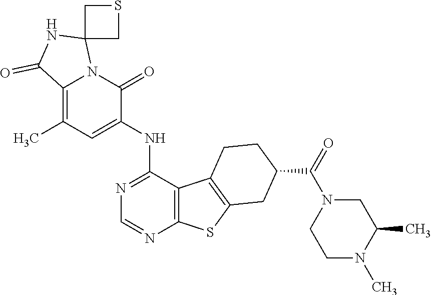 [SMILES: Cc1cc(Nc2ncnc3sc4c(c23)CC[C@H](C(=O)N2CCN(C)[C@H](C)C2)C4)c(=O)n2c1C(=O)NC21CSC1]

A mixture of 50 mg (106 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 3.0 mL N,N-dimethylacetamide, 278 μL N-ethyl-N-isopropylpropan-2-amine, 99.6 mg (2R)-1,2-dimethylpiperazine dihydrochloride and 190 μL 2,4,6-tripropyl-1,3,5,2,4,6-trioxatriphosphinane 2,4,6-trioxide solution (50% in N,N-dimethylformamide) was stirred at RT overnight. Another 59.8 mg (2R)-1,2-dimethylpiperazine dihydrochloride were added and stirring continued at 50° C. for 3 hours. Water was added, the precipitate was washed with water, ethanol and diethylether and purified by flash chromatography (Biotage SNAP cartridge NH 11 g, methanol:dichloromethane) to give 32 mg (53%) of the title compound.

LC-MS: m/z=566.5 [M+H]⁺.

¹H-NMR (400 MHz, DMSO-d6), δ [ppm]=1.00+1.04 (3H), 1.76-2.24 (5H), 2.19 (3H), 2.46 (3H), 2.70-3.12 (4H), 3.17-3.26 (1H), 3.28 (2H), 3.31 (2H), 3.92 (1H), 4.17 (1H), 4.67 (2H), 8.66 (1H), 8.75 (1H), 9.01 (1H), 10.72 (1H).

### Example 51

### 8-Methyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione

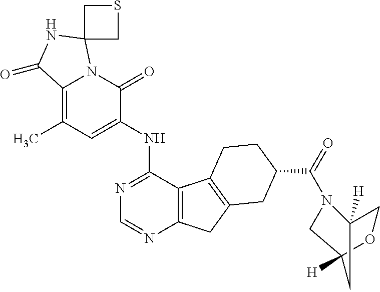 [SMILES: Cc1cc(Nc2ncnc3c2C2=C(C3)C[C@@H](C(=O)N3C[C@H]4C[C@@H]3CO4)CC2)c(=O)n2c1C(=O)NC21CSC1]

A mixture of 50 mg (106 μmol) (7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 44), 2.9 mL N,N-dimethylacetamide, 167 μL N-ethyl-N-isopropylpropan-2-amine, 72.2 mg (1R,4R)-2-oxa-5-azabicyclo[2.2.1]heptane hydrochloride and 190 μL 2,4,6-tripropyl-1,3,5,2,4,6-trioxatriphosphinane 2,4,6-trioxide solution (50% in N,N-dimethylformamide) was stirred at RT overnight. Another 14.4 mg (1R,4R)-2-oxa-5-azabicyclo[2.2.1]heptane hydrochloride were added and stirring continued at 50° C. for 3 hours. Water was added, the precipitate was washed with water, ethanol and diethylether and purified by flash chromatography (Biotage SNAP cartridge NH 11 g, methanol:dichloromethane) to give 35 mg (60%) of the title compound.

LC-MS: m/z=551.5 [M+H]⁺.

¹H-NMR (400 MHz, DMSO-d6), δ [ppm]=1.75-1.95 (3H), 2.20+2.28 (1H), 2.46 (3H), 2.79-3.31 (8H), 3.55-3.80 (3H), 4.61-4.72 (3H), 4.78+4.93 (1H), 8.66 (1H), 8.73+8.75 (1H), 9.01+9.03 (1H), 10.72 (1H).

### Example 52

### (7S)-4-[(4,4-Difluoro-8′-methyl-1′,5′-dioxo-1′,5′-dihydro-2′H-spiro[cyclohexane-1,3′-imidazo[1,5-a]pyridin]-6′-yl)amino]-N,N-dimethyl-5,6,7,8-tetrahydro[1]benzothieno[2,3-d]pyrimidine-7-carboxamide

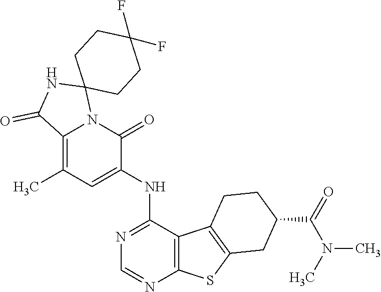 [SMILES: Cc1cc(Nc2ncnc3sc4c(c23)CC[C@H](C(=O)N(C)C)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 122 μL N-ethyl-N-isopropylpropan-2-amine, 291 μL N-methylmethanamine (2M in tetrahydrofuran) and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 57 mg (85%) of the title compound.

LC-MS: m/z=543.5 [M+H]⁺.

¹H-NMR (400 MHz, DMSO-d6), δ [ppm]=1.68 (2H), 1.80 (1H), 2.13-2.37 (5H), 2.50 (3H*), 2.84-3.06 (2H), 2.68 (3H), 3.11 (3H), 3.14-3.30 (5H), 8.64 (1H), 8.76 (1H), 8.97 (1H), 10.39 (1H);

*: at least partially hidden by solvent peak.

### Example 53

### 6′-{[(7S)-7-(Azetidin-1-ylcarbonyl)-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione

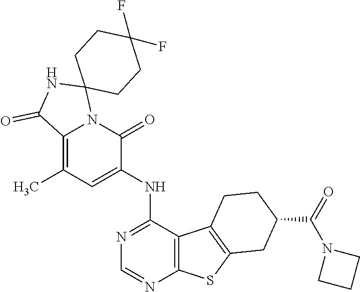 [SMILES: Cc1cc(Nc2ncnc3sc4c(c23)CC[C@H](C(=O)N2CCC2)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 162 μL N-ethyl-N-isopropylpropan-2-amine, 43.6 mg azetidine hydrochloride and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 62 mg (91%) of the title compound.

LC-MS: m/z=555.5 [M+H]⁺.

¹H-NMR (400 MHz, DMSO-d6), δ [ppm]=1.68 (2H), 1.81 (1H), 2.09-2.29 (6H), 2.50 (3H*), 2.77 (1H), 2.92 (2H), 3.13-3.30 (5H*), 3.89 (2H), 4.26 (2H), 8.64 (1H), 8.77 (1H), 8.98 (1H), 10.39 (1H);

*: at least partially hidden by solvent peak.

### Example 54

### 1-({(7S)-4-[(4,4-Difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile

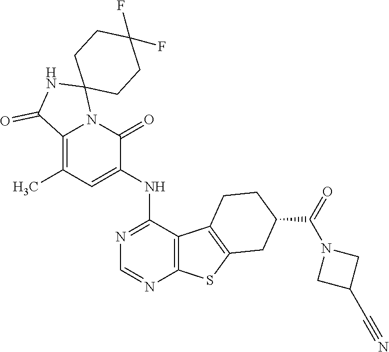 [SMILES: Cc1cc(Nc2ncnc3sc4c(c23)CC[C@H](C(=O)N2CC(C#N)C2)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 182 μL N-ethyl-N-isopropylpropan-2-amine, 69.0 mg azetidine-3-carbonitrile hydrochloride and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 63 mg (89%) of the title compound.

LC-MS: m/z=580.5 [M+H]⁺.

¹H-NMR (400 MHz, DMSO-d6), δ [ppm]=1.68 (2H), 1.80 (1H), 2.13-2.36 (5H), 2.50 (3H*), 2.77 (1H), 2.86-3.04 (2H), 3.14-3.27 (4H*), 3.82 (1H), 4.05 (1H), 4.19 (1H), 4.42-4.62 (2H), 8.64 (1H), 8.76 (1H), 8.97 (1H), 10.39 (1H);

*: at least partially hidden by solvent peak.

### Example 55

### 6′-{[(7S)-7-{[3-(Dimethylamino)azetidin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione

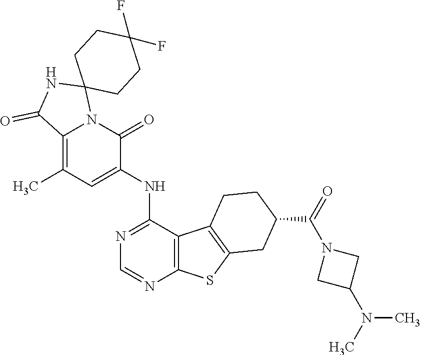 [SMILES: Cc1cc(Nc2ncnc3sc4c(c23)CC[C@H](C(=O)N2CC(N(C)C)C2)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 304 μL N-ethyl-N-isopropylpropan-2-amine, 101 mg N,N-dimethylazetidin-3-amine dihydrochloride and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 55 mg (75%) of the title compound.

LC-MS: m/z=598.6 [M+H]⁺.

¹H-NMR (400 MHz, DMSO-d6), δ [ppm]=1.68 (2H), 1.80 (1H), 2.08 (3H), 2.10 (3H), 2.12-2.37 (6H), 2.50 (3H*), 2.79 (1H), 2.92 (2H), 3.05 (1H), 3.13-3.30 (3H*), 3.67 (1H), 3.88 (1H), 4.03+4.10 (1H), 4.24+4.29 (1H), 8.64 (1H), 8.76 (1H), 8.96 (1H), 10.39 (1H);

*: at least partially hidden by solvent peak.

### Example 56

### 6′-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione

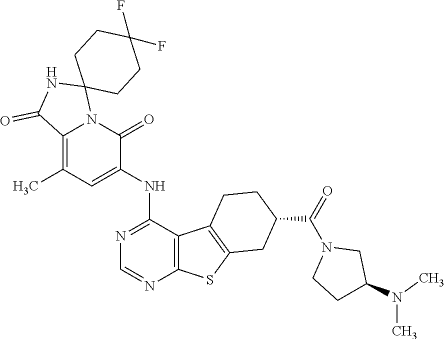 [SMILES: Cc1cc(Nc2ncnc3sc4c(c23)CC[C@H](C(=O)N2CC[C@H](N(C)C)C2)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 122 μL N-ethyl-N-isopropylpropan-2-amine, 73.9 μL (S)-3-(dimethylamino)pyrrolidine and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 59 mg (78%) of the title compound.

LC-MS: m/z=610.5 [M−H]⁺.

¹H-NMR (400 MHz, DMSO-d6), δ [ppm]=1.68 (2H), 1.79 (1H), 2.02+2.09 (1H), 2.12-2.37 (5H), 2.16 (3H), 2.17 (3H), 2.50 (3H*), 2.61+2.74 (1H), 3.17-3.66 (11H*), 3.80+3.89 (1H), 8.64 (1H), 8.76+8.77 (1H), 8.97+8.98 (1H), 10.39 (1H);

*: at least partially hidden by solvent peak.

### Example 57

### 6′-{[(7S)-7-{[(3R)-3,4-Dimethylpiperazin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione

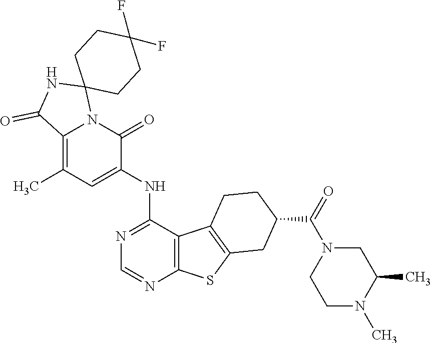 [SMILES: Cc1cc(Nc2ncnc3sc4c(c23)CC[C@H](C(=O)N2CCN(C)[C@H](C)C2)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 304 μL N-ethyl-N-isopropylpropan-2-amine, 109 mg (2R)-1,2-dimethylpiperazine dihydrochloride and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 63 mg (84%) of the title compound.

LC-MS: m/z=612.6 [M+H]⁺.

¹H-NMR (400 MHz, DMSO-d6), δ [ppm]=0.97-1.07 (3H), 1.68 (2H), 1.75-2.37 (8H), 2.19 (3H), 2.52 (3H*), 2.70-3.11 (4H), 3.17-3.41 (6H*), 3.91 (1H), 4.15 (1H), 8.65 (1H), 8.77 (1H), 8.98 (1H), 10.39 (1H);

*: at least partially hidden by solvent peak.

### Example 58

### 4,4-Difluoro-8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione

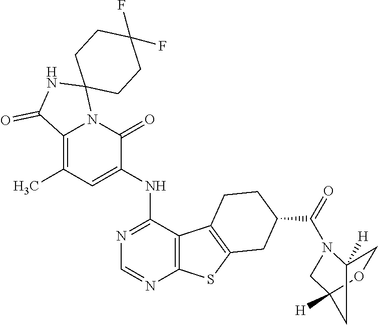 [SMILES: Cc1cc(Nc2ncnc3sc4c(c23)CC[C@H](C(=O)N2C[C@H]3C[C@@H]2CO3)C4)c(=O)n2c1C(=O)NC21CCC(F)(F)CC1]

A mixture of 60 mg (116 μmol) (7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 42), 4.0 mL N,N-dimethylacetamide, 182 μL N-ethyl-N-isopropylpropan-2-amine, 77.1 mg (1R,4R)-2-oxa-5-azabicyclo[2.2.1]heptane hydrochloride and 208 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 65 mg (89%) of the title compound.

LC-MS: m/z=597.5 [M+H]⁺.

¹H-NMR (400 MHz, DMSO-d6), δ [ppm]=1.69 (2H), 1.76-1.96 (3H), 2.12-2.37 (5H), 2.50 (3H*), 2.75-3.11 (3H), 3.17-3.36 (5H), 3.51-3.79 (3H), 4.61+4.66 (1H), 4.78+4.92 (1H), 8.64 (1H), 8.75 (1H), 8.98 (1H), 10.39 (1H);

*: at least partially hidden by solvent peak.

### Example 59

### (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid

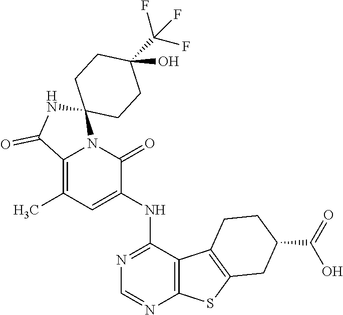 [SMILES: Cc1cc(Nc2ncnc3sc4c(c23)CC[C@H](C(=O)O)C4)c(=O)n2c1C(=O)N[C@]21CC[C@@](O)(C(F)(F)F)CC1]

A mixture of 278 mg (470 μmol) ethyl (7S)-4-{[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylate (prepared according to example 59a) in 7.5 mL tetrahydrofuran, 3.0 mL ethanol and 2.8 mL lithium hydroxide solution (1M in water) was stirred at RT overnight. Water was added and the mixture was acidified with hydrochloric acid. After concentration the precipitate was washed with water and dried to give 258 mg (93%) of the title compound.

LC-MS: m/z=564.5 [M+H]⁺.

¹H-NMR (400 MHz, DMSO-d6), δ [ppm]=1.43 (2H), 1.82 (2H), 1.90-2.08 (3H), 2.23 (1H), 2.52 (3H*), 2.89 (1H), 3.01 (1H), 3.10 (1H), 3.16-3.53 (4H), 6.05 (1H), 8.64 (1H), 8.74 (1H), 9.02 (1H), 10.41 (1H), 12.52 (1H);

*: at least partially hidden by solvent peak.

### Example 59a

### Ethyl (7S)-4-{[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylate

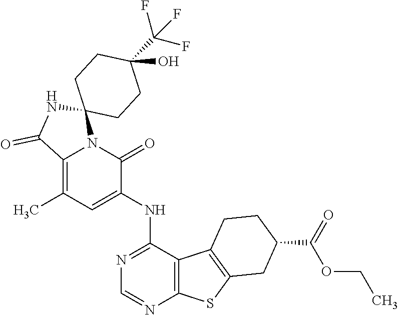 [SMILES: CCOC(=O)[C@H]1CCc2c(sc3ncnc(Nc4cc(C)c5n(c4=O)[C@]4(CC[C@](O)(C(F)(F)F)CC4)NC5=O)c23)C1]

To a solution of 232 mg (836 μmol) ethyl (7S)-4-amino-5,6,7,8-tetrahydro[1]benzothieno[2,3-d]pyrimidine-7-carboxylate (prepared according to example 1 b) and 331 mg (836 μmol) (cis)-6′-bromo-4-hydroxy-8′-methyl-4-(trifluoromethyl)-2′H-spiro[cyclohexane-1,3′-imidazo[1,5-a]pyridine]-1′,5′-dione (prepared according to example 59b) in 30 mL 1,4-dioxane were added 818 mg cesium carbonate and the mixture was degassed and purged with argon several times. 51.8 mg 4,5-bis(diphenylphosphino)-9,9-dimethylxanthene, 42.7 mg 2-(dicyclohexyl-phosphino)-2′,4′,6′-triisopropylbiphenyl, 20 mg palladium(II)acetate and 82 mg tris(dibenzylideneacetone)dipalladium(0) were added and the mixture was stirred at 105° C. for 2 hours. Dichloromethane and ethanol were added, the precipitate was filtered off, the filtrate was concentrated and the residue crystallized from ethanol to give 280 mg (57%) of the title compound.

LC-MS: m/z=592.5 [M+H]⁺.

### Example 59b

### (cis)-6′-Bromo-4-hydroxy-8′-methyl-4-(trifluoromethyl)-2′H-spiro[cyclohexane-1,3′-imidazo[1,5-a]pyridine]-1′,5′-dione (A) and (cis)-6′-Bromo-4-hydroxy-8′-methyl-4-(trifluoromethyl)-2′H-spiro[cyclohexane-1,3′-imidazo[1,5-a]pyridine]-1′,5′-dione (B)

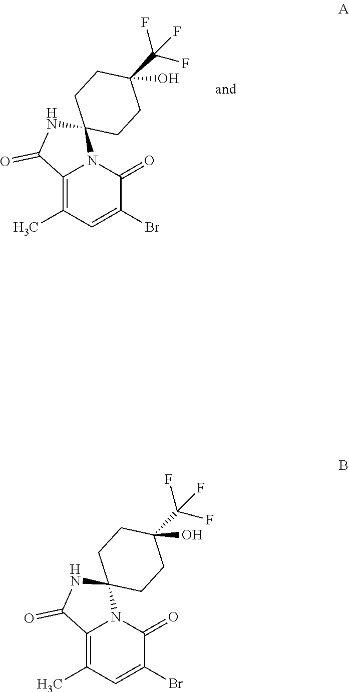 [SMILES: Cc1cc(Br)c(=O)n2c1C(=O)N[C@]21CC[C@](O)(C(F)(F)F)CC1 ; Cc1cc(Br)c(=O)n2c1C(=O)N[C@]21CC[C@](O)(C(F)(F)F)CC1]

To a solution of 1.18 g (5.12 mmol) 5-bromo-3-methyl-6-oxo-1,6-dihydropyridine-2-carbaldehyde (prepared according to PCT Int. Appl. (2015), WO 2015200481) and 3.73 g (20.5 mmol) 4-hydroxy-4-(trifluoromethyl)cyclohexanone (CAS-No: 120929-90-0) in 14 mL 1,4-dioxane were added 135 μl sulfuric acid and the mixture was stirred at 95° C. for 3 hours.

After concentration water was added, the precipitate washed with water and diethyl ether and dried to give 1.78 g (88%) of a mixture of title compounds A and B.

LC-MS: m/z=395.2 [M+H]⁺.

The isomeric mixture was separated by flash chromatography (Biotage SNAP Ultra cartridge silica 100 g, ethyl acetate:n-hexane) to give trans-isomer A and cis-isomer B.

### Example 60

### (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-N,N-dimethyl-5,6,7,8-tetrahydro[1]benzothieno[2,3-d]pyrimidine-7-carboxamide

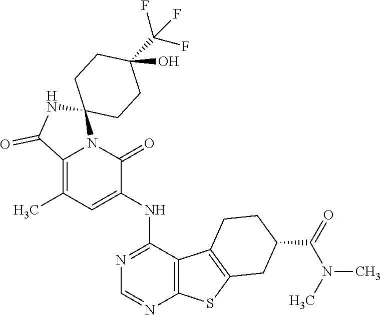 [SMILES: Cc1cc(Nc2ncnc3sc4c(c23)CC[C@H](C(=O)N(C)C)C4)c(=O)n2c1C(=O)N[C@]21CC[C@@](O)(C(F)(F)F)CC1]

A mixture of 50 mg (89 μmol) (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 4.0 mL N,N-dimethylacetamide, 93 μL N-ethyl-N-isopropylpropan-2-amine, 222 μL N-methylmethanamine (2M in tetrahydrofuran) and 158 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 47 mg (85%) of the title compound.

LC-MS: m/z=591.6 [M+H]⁺.

¹H-NMR (400 MHz, DMSO-d6), δ [ppm]=1.44 (2H), 1.76-1.89 (3H), 1.96 (2H), 2.15 (1H), 2.52 (3H*), 2.88 (3H), 2.89-3.05 (2H), 3.11 (3H), 3.16-3.30 (3H), 3.36-3.49 (2H), 6.04 (1H), 8.64 (1H), 8.76 (1H), 9.02 (1H), 10.42 (1H);

*: at least partially hidden by solvent peak.

### Example 61

### (cis)-6′-{[(7S)-7-(Azetidin-1-ylcarbonyl)-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione

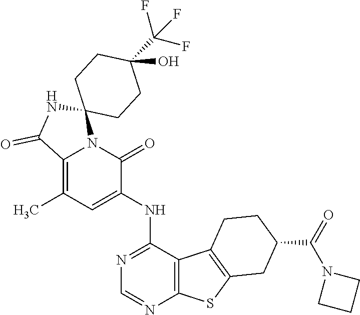 [SMILES: Cc1cc(Nc2ncnc3sc4c(c23)CC[C@H](C(=O)N2CCC2)C4)c(=O)n2c1C(=O)N[C@]21CC[C@@](O)(C(F)(F)F)CC1]

A mixture of 50 mg (89 μmol) (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 4.0 mL N,N-dimethylacetamide, 124 μL N-ethyl-N-isopropylpropan-2-amine, 33.2 mg azetidine hydrochloride and 158 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 47 mg (87%) of the title compound.

LC-MS: m/z=603.5 [M+H]⁺.

¹H-NMR (400 MHz, DMSO-d6), δ [ppm]=1.33-1.54 (6H), 1.74-2.03 (7H), 2.13 (1H), 2.21 (2H), 2.50 (3H*), 2.77 (1H), 2.92 (2H), 3.89 (1H), 4.26 (1H), 6.04 (1H), 8.64 (1H), 8.76 (1H), 9.01 (1H), 10.42 (1H);

*: at least partially hidden by solvent peak.

### Example 62

### (cis)-6′-{[(7S)-7-{[3-(Dimethylamino)-1,3-diazetidin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione

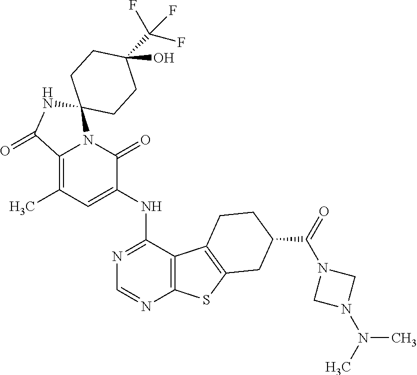 [SMILES: Cc1cc(Nc2ncnc3sc4c(c23)CC[C@H](C(=O)N2CN(N(C)C)C2)C4)c(=O)n2c1C(=O)N[C@]21CC[C@@](O)(C(F)(F)F)CC1]

A mixture of 50 mg (89 μmol) (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 4.0 mL N,N-dimethylacetamide, 232 μL N-ethyl-N-isopropylpropan-2-amine, 76.8 mg N,N-dimethylazetidin-3-amine dihydrochloride and 158 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 35 mg (61%) of the title compound.

LC-MS: m/z=646.6 [M+H]⁺.

¹H-NMR (400 MHz, DMSO-d6), δ [ppm]=1.39-1.53 (4H), 1.83 (3H), 1.96 (2H), 2.09 (3H), 2.10 (3H), 2.50 (3H*), 2.80 (1H), 2.85-3.25 (6H*), 3.67 (1H), 3.88 (1H), 4.06 (1H), 4.26 (1H), 6.04 (1H), 8.64 (1H), 8.76 (1H), 9.01 (1H), 10.42 (1H);

*: at least partially hidden by solvent peak.

### Example 63

### (cis)-6′-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione

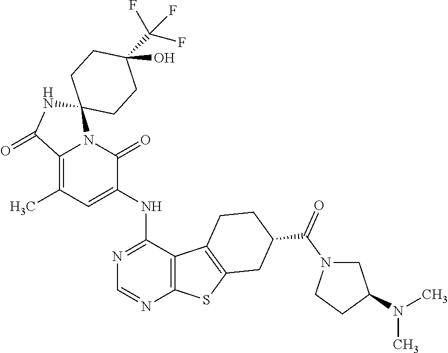 [SMILES: Cc1cc(Nc2ncnc3sc4c(c23)CC[C@H](C(=O)N2CC[C@H](N(C)C)C2)C4)c(=O)n2c1C(=O)N[C@]21CC[C@@](O)(C(F)(F)F)CC1]

A mixture of 50 mg (89 μmol) (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 4.0 mL N,N-dimethylacetamide, 93 μL N-ethyl-N-isopropylpropan-2-amine, 56.3 μL (S)-3-(dimethylamino)pyrrolidine and 158 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 54 mg (92%) of the title compound.

LC-MS: m/z=660.6 [M+H]⁺.

¹H-NMR (400 MHz, DMSO-d6), δ [ppm]=1.38-1.49 (3H), 1.57-2.13 (8H), 2.17 (3H), 2.18 (3H), 2.50 (3H*), 2.62+2.74 (1H), 2.91-3.06 (3H), 3.16-3.66 (6H*), 3.78+3.87 (1H), 6.03 (1H), 8.64 (1H), 8.76 (1H), 9.02 (1H), 10.42 (1H);

*: at least partially hidden by solvent peak.

### Example 64

### (cis)-4-Hydroxy-8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-4-(trifluoromethyl)-2′H-spiro[cyclohexane-1,3′-imidazo[1,5-a]pyridine]-1′,5′-dione

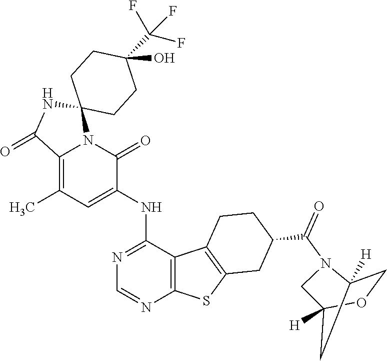 [SMILES: Cc1cc(Nc2ncnc3sc4c(c23)CC[C@H](C(=O)N2C[C@H]3C[C@@H]2CO3)C4)c(=O)n2c1C(=O)N[C@]21CC[C@@](O)(C(F)(F)F)CC1]

A mixture of 50 mg (89 μmol) (7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 4.0 mL N,N-dimethylacetamide, 139 μL N-ethyl-N-isopropylpropan-2-amine, 60.2 mg (1R,4R)-2-oxa-5-azabicyclo[2.2.1]heptane hydrochlorid and 158 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 50 mg (88%) of the title compound.

LC-MS: m/z=645.5 [M+H]⁺.

¹H-NMR (400 MHz, DMSO-d6), δ [ppm]=1.44 (2H), 1.75-2.04 (7H), 2.13+2.21 (1H), 2.50 (3H*), 2.80-2.87 (1H), 2.92-3.77 (10H), 4.61+4.66 (1H), 4.77+4.91 (1H), 6.01+6.03 (1H), 8.64 (1H), 8.74+8.75 (1H), 9.03+9.04 (1H), 10.42 (1H);

*: at least partially hidden by solvent peak.

### Example 65

### (7S)-4-[(8-Methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid

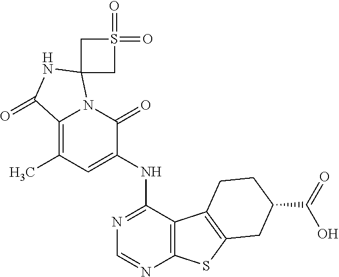 [SMILES: Cc1cc(Nc2ncnc3sc4c(c23)CC[C@H](C(=O)O)C4)c(=O)n2c1C(=O)NC21CS(=O)(=O)C1]

A mixture of 675 mg (1.28 mmol) ethyl (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate (prepared according to example 65a) in 19 mL tetrahydrofuran, 9 mL ethanol and 7.6 mL lithium hydroxide solution (1M in water) was stirred at RT overnight. Water was added and the mixture was acidified with hydrochloric acid. After concentration the precipitate was washed with water and dried to give 604 mg (94%) of the title compound.

LC-MS: m/z=502.4 [M+H]⁺.

¹H-NMR (400 MHz, DMSO-d6), δ [ppm]=2.03 (1H), 2.27 (1H), 2.48 (3H), 2.92 (1H), 3.02 (1H), 3.11 (1H), 3.24 (2H), 4.39 (2H), 5.65 (2H), 8.66 (1H), 8.78 (1H), 8.90 (1H), 10.62 (1H), 12.51 (1H)

### Example 65a

### Ethyl (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate

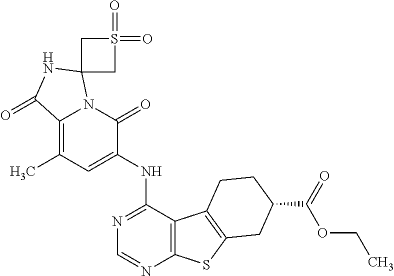 [SMILES: CCOC(=O)[C@H]1CCc2c(sc3ncnc(Nc4cc(C)c5n(c4=O)C4(CS(=O)(=O)C4)NC5=O)c23)C1]

To a solution of 514 mg (1.85 mmol) ethyl (7S)-4-amino-5,6,7,8-tetrahydro[1]benzothieno[2,3-d]pyrimidine-7-carboxylate (prepared according to example 1 b) and 617 mg (1.85 mmol) 6-bromo-8-methyl-2H-spiro[imidazo[1,5-a]pyridine-3,3′-thietane]-1,5-dione 1′,1′-dioxide (prepared according to example 65b) in 51 mL 1,4-dioxane were added 1.81 g cesium carbonate and the mixture was degassed and purged with argon several times. 115 mg 4,5-bis(diphenylphosphino)-9,9-dimethylxanthene, 94.4 mg 2-(dicyclohexyl-phosphino)-2′,4′,6′-triisopropylbiphenyl, 44.5 mg palladium(II)acetate and 181 mg tris(dibenzylideneacetone)dipalladium(0) were added and the mixture was stirred at 100° C. for 2 hours. After concentration the residue was purified by flash chromatography (Biotage SNAP cartridge silica 100 g, ethanol:dichloromethane) and crystallization from ethanol to give 675 mg (69%) of the title compound.

LC-MS: m/z=530.5 [M+H]⁺.

### Example 65b

### 6-Bromo-8-methyl-2H-spiro[imidazo[1,5-a]pyridine-3,3′-thietane]-1,5-dione 1′,1′-dioxide

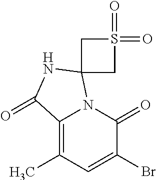 [SMILES: Cc1cc(Br)c(=O)n2c1C(=O)NC21CS(=O)(=O)C1]

To a solution of 400 mg (1.73 mmol) 5-bromo-3-methyl-6-oxo-1,6-dihydropyridine-2-carbaldehyde (prepared according to PCT Int. Appl. (2015), WO 2015200481) and 312 mg (2.60 mmol) thietan-3-one 1,1-dioxide in 4.8 mL 1,4-dioxane were added 46 μl sulfuric acid and the mixture was stirred at 95° C. for 4 hours. After concentration water was added, the precipitate washed with water and diethyl ether and dried to give 420 mg (73%) of the title compound.

LC-MS: m/z=333.3 [M+H]⁺.

### Example 66

### (7S)—N,N-Dimethyl-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxamide

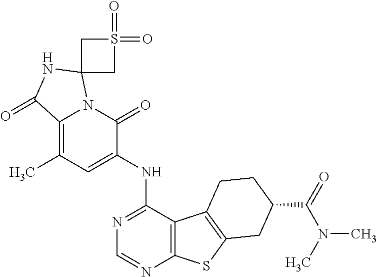 [SMILES: Cc1cc(Nc2ncnc3sc4c(c23)CC[C@H](C(=O)N(C)C)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 146 μL N-ethyl-N-isopropylpropan-2-amine, 349 μL N-methylmethanamine (2M in tetrahydrofuran) and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 45 mg (58%) of the title compound.

LC-MS: m/z=529.5 [M+H]⁺.

¹H-NMR (400 MHz, DMSO-d6), Shift [ppm]=1.84 (1H), 2.17 (1H), 2.48 (3H), 2.88 (3H), 2.90-3.07 (2H), 3.12 (3H), 3.18-3.30 (3H), 4.39 (2H), 5.63 (2H), 8.67 (1H), 8.81 (1H), 8.90 (1H), 10.62 (1H).

### Example 67

### 6-{[(7S)-7-(Azetidin-1-ylcarbonyl)-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide

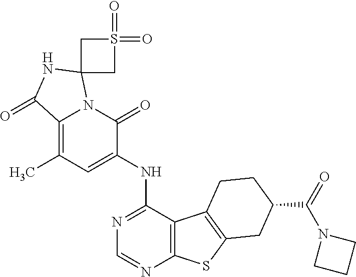 [SMILES: Cc1cc(Nc2ncnc3sc4c(c23)CC[C@H](C(=O)N2CCC2)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 219 μL N-ethyl-N-isopropylpropan-2-amine, 65.3 mg azetidine hydrochloride and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 36 mg (48%) of the title compound.

LC-MS: m/z=541.5 [M+H]⁺.

¹H-NMR (400 MHz, DMSO-d6), Shift [ppm]=1.83 (1H), 2.10-2.28 (3H), 2.48 (3H), 2.79 (1H), 2.93 (2H), 3.12-3.28 (2H), 3.89 (2H), 4.27 (2H), 4.39 (2H), 5.63 (2H), 8.66 (1H), 8.80 (1H), 8.89 (1H), 10.62 (1H).

### Example 68

### 1-({(7S)-4-[(8-Methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidin-7-yl}carbonyl)azetidine-3-carbonitrile

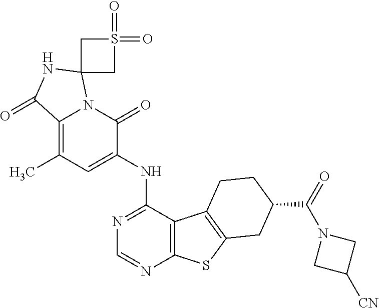 [SMILES: Cc1cc(Nc2ncnc3sc4c(c23)CC[C@H](C(=O)N2CC(C#N)C2)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 219 μL N-ethyl-N-isopropylpropan-2-amine, 82.7 mg azetidine-3-carbonitrile hydrochloride and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and crystallized from ethanol to give 42 mg (51%) of the title compound.

LC-MS: m/z=566.5 [M+H]⁺.

¹H-NMR (400 MHz, DMSO-d6), Shift [ppm]=1.83 (1H), 2.18 (1H), 2.48 (3H), 2.80 (1H), 2.85-3.04 (2H), 3.16-3.29 (2H), 3.82 (1H), 4.05 (1H), 4.19 (1H), 4.39 (2H), 4.44-4.63 (2H), 5.63 (2H), 8.67 (1H), 8.80 (1H), 8.90 (1H), 10.62 (1H).

### Example 69

### 6-{[(7S)-7-{[3-(Dimethylamino)azetidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide

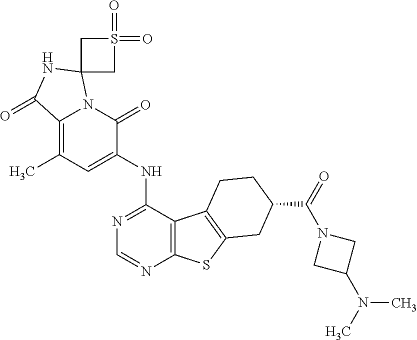 [SMILES: Cc1cc(Nc2ncnc3sc4c(c23)CC[C@H](C(=O)N2CC(N(C)C)C2)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 365 μL N-ethyl-N-isopropylpropan-2-amine, 121 mg N,N-dimethylazetidin-3-amine dihydrochloride and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 45 mg (52%) of the title compound.

LC-MS: m/z=584.5 [M+H]⁺.

¹H-NMR (400 MHz, DMSO-d6), Shift [ppm]=1.83 (1H), 2.18 (1H), 2.48 (3H), 2.52 (6H*), 2.79 (1H), 2.87-3.04 (2H), 3.14-3.29 (2H), 3.82 (1H), 4.05 (1H), 4.19 (1H), 4.39 (2H), 4.46-4.63 (2H), 5.63 (2H), 8.67 (1H), 8.80 (1H), 8.90 (1H), 10.62 (1H).

*: at least partially hidden by solvent peak.

### Example 70

### 6-{[(7S)-7-{[(3S)-3-(Dimethylamino)pyrrolidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide

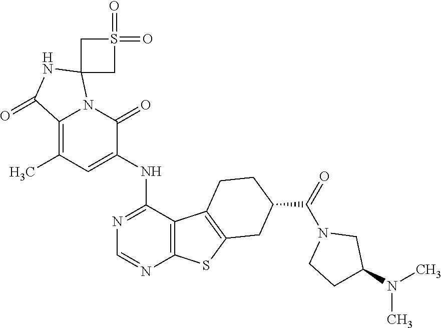 [SMILES: Cc1cc(Nc2ncnc3sc4c(c23)CC[C@H](C(=O)N2CC[C@H](N(C)C)C2)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 146 μL N-ethyl-N-isopropylpropan-2-amine, 88.6 μL (S)-3-(dimethylamino)pyrrolidine and 249 μL 2,4,6-tripropyl-1,3,5,2,4,6-trioxatriphosphinane 2,4,6-trioxide solution (50% in N,N-dimethylformamide) was stirred at 50° C. for 2.5 days. After concentration the residue was purified by flash chromatography (Biotage SNAP cartridge NH 11 g, methanol:dichloromethane) to give 50 mg (57%) of the title compound.

LC-MS: m/z=598.6 [M+H]⁺.

¹H-NMR (400 MHz, DMSO-d6), Shift [ppm]=1.58-1.92 (2H), 1.99-2.26 (2H), 2.18 (6H), 2.50 (3H*), 2.61+2.75 (1H), 2.91-3.67 (8H), 3.80+3.89 (1H), 4.38 (2H), 5.62 (2H), 8.67 (1H), 8.81 (1H), 8.90 (1H), 10.61 (1H);

*: at least partially hidden by solvent peak.

### Example 71

### 6-{[(7S)-7-{[(3R)-3,4-Dimethylpiperazin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide

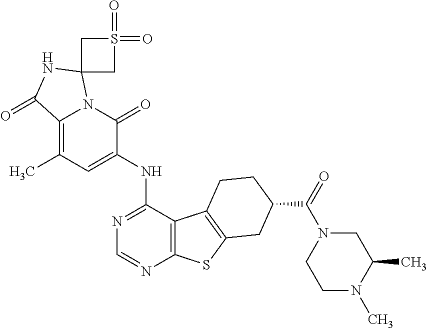 [SMILES: Cc1cc(Nc2ncnc3sc4c(c23)CC[C@H](C(=O)N2CCN(C)[C@H](C)C2)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 365 μL N-ethyl-N-isopropylpropan-2-amine, 131 mg (2R)-1,2-dimethylpiperazine dihydrochloride and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 33 mg (36%) of the title compound.

LC-MS: m/z=598.6 [M+H]⁺.

¹H-NMR (400 MHz, DMSO-d6), Shift [ppm]=1.17-1.39 (3H), 1.85 (1H), 2.19 (1H), 2.40-2.61 (1H), 2.52 (3H*), 2.69-3.68 (12H*), 4.21-4.61 (4H), 5.61 (2H), 8.68 (1H), 8.82 (1H), 8.90 (1H), 10.64 (1H)

*: at least partially hidden by solvent peak.

### Example 72

### 8-Methyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione 1′,1′-dioxide

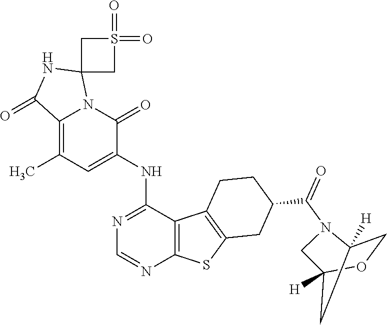 [SMILES: Cc1cc(Nc2ncnc3sc4c(c23)CC[C@H](C(=O)N2C[C@H]3C[C@@H]2CO3)C4)c(=O)n2c1C(=O)NC21CS(=O)(=O)C1]

A mixture of 70 mg (140 μmol) (7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 65), 4.0 mL N,N-dimethylacetamide, 219 μL N-ethyl-N-isopropylpropan-2-amine, 94.6 mg (1R,4R)-2-oxa-5-azabicyclo[2.2.1]heptane hydrochloride and 249 μL 2,4,6-tripropyl-1,3,5,2,4,6-trioxatriphosphinane 2,4,6-trioxide solution (50% in N,N-dimethylformamide) was stirred at 50° C. for 2.5 days. Water was added, the precipitate was washed with water, ethanol and diethylether and dried to give 63 mg (74%) of the title compound.

LC-MS: m/z=583.6 [M+H]⁺.

¹H-NMR (400 MHz, DMSO-d6), Shift [ppm]=1.73-1.99 (3H), 2.16+2.24 (1H), 2.50 (3H*), 2.79-3.41 (6H), 3.53-3.82 (3H), 4.39 (2H), 4.61+4.67 (1H), 4.78+4.93 (1H), 5.63 (2H), 8.67 (1H), 8.79+8.82 (1H), 8.92 (1H), 10.63 (1H);

*: at least partially hidden by solvent peak.

### Example 73

### (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid

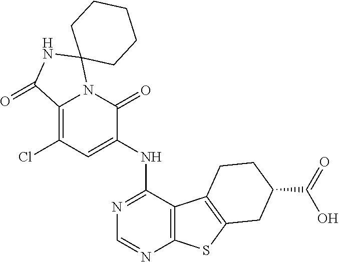 [SMILES: O=C1NC2(CCCCC2)n2c1c(Cl)cc(Nc1ncnc3sc4c(c13)CC[C@H](C(=O)O)C4)c2=O]

A mixture of 632 mg (1.20 mmol) ethyl (7S)-4-[(8′-chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylate (prepared according to example 73a) in 18 mL tetrahydrofuran, 9 mL ethanol and 7.2 mL lithium hydroxide solution (1M in water) was stirred at RT overnight. Water was added and the mixture was acidified with hydrochloric acid. After concentration the precipitate was washed with water and dried to give 598 mg (95%) of the title compound.

LC-MS: m/z=500.4 [M+H]⁺.

¹H-NMR (400 MHz, DMSO-d6), δ [ppm]=1.29 (1H), 1.57 (2H), 1.60-1.72 (3H), 1.72-1.83 (2H), 2.01 (1H), 2.25 (1H), 2.83-3.23 (7H), 8.68 (1H), 8.85 (1H), 8.96 (1H), 10.40 (1H), 12.58 (1H).

### Example 73a

### Ethyl (7S)-4-[(8′-chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylate

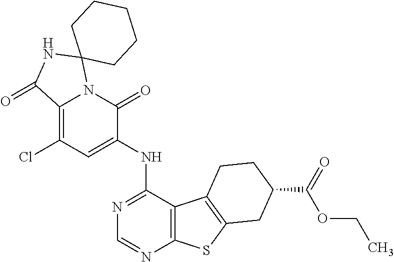 [SMILES: CCOC(=O)[C@H]1CCc2c(sc3ncnc(Nc4cc(Cl)c5n(c4=O)C4(CCCCC4)NC5=O)c23)C1]

To a solution of 500 mg (1.80 mmol) ethyl (7S)-4-amino-5,6,7,8-tetrahydro[1]benzothieno[2,3-d]pyrimidine-7-carboxylate (prepared according to example 1 b) and 658 mg (1.98 mmol) 6′-bromo-8′-chloro-2′H-spiro[cyclohexane-1,3′-imidazo[1,5-a]pyridine]-1′,5′-dione (PCT Int. Appl. (2017), WO 2017087808) in 70 mL 1,4-dioxane were added 1.76 g cesium carbonate and the mixture was degassed and purged with argon several times. 112 mg 4,5-bis(diphenylphosphino)-9,9-dimethylxanthene, 92 mg 2-(dicyclohexyl-phosphino)-2′,4′,6′-triisopropylbiphenyl, 43.3 mg palladium(II)acetate and 177 mg tris(dibenzylideneacetone)dipalladium(0) were added and the mixture was stirred at 100° C. for 2 hours. After concentration the residue was purified by flash chromatography (Biotage SNAP cartridge silica 100 g, ethanol:dichloromethane) and crystallization from ethanol to give 634 mg (67%) of the title compound.

LC-MS: m/z=528.4 [M+H]⁺.

### Example 74

### (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide

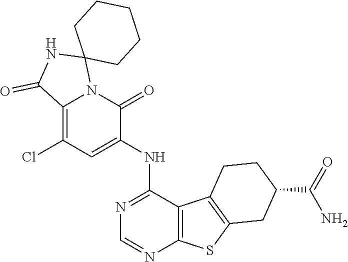 [SMILES: NC(=O)[C@H]1CCc2c(sc3ncnc(Nc4cc(Cl)c5n(c4=O)C4(CCCCC4)NC5=O)c23)C1]

A mixture of 80 mg (160 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 5.0 mL N,N-dimethylacetamide, 167 μL N-ethyl-N-isopropylpropan-2-amine, 5.0 mL ammonia (0.4M in tetrahydrofurane) and 286 μL 2,4,6-tripropyl-1,3,5,2,4,6-trioxatriphosphinane 2,4,6-trioxide solution (50% in N,N-dimethylformamide) was stirred at 60° C. overnight. Water was added, the precipitate was washed with water and ethanol and dried to give 56 mg (66%) of the title compound.

LC-MS: m/z=499.1 [M+H]⁺.

¹H-NMR (400 MHz, DMSO-d6), δ [ppm]=1.29 (1H), 1.57 (2H), 1.66 (3H), 1.72-1.81 (2H), 1.91 (1H), 2.21 (1H), 2.68 (1H), 2.88-3.29 (6H), 6.99 (1H), 7.48 (1H), 8.68 (1H), 8.85 (1H), 8.98 (1H), 10.40 (1H).

### Example 75

### (7S)-4-[(8′-Chloro-1′,5′-dioxo-1′,5′-dihydro-2′H-spiro[cyclohexane-1,3′-imidazo[1,5-a]pyridin]-6′-yl)amino]-N,N-dimethyl-5,6,7,8-tetrahydro[1]benzothieno[2,3-d]pyrimidine-7-carboxamide

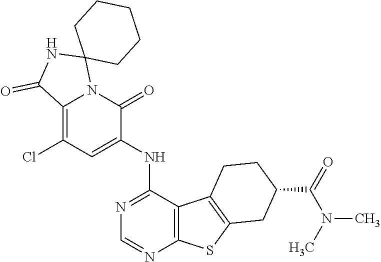 [SMILES: CN(C)C(=O)[C@H]1CCc2c(sc3ncnc(Nc4cc(Cl)c5n(c4=O)C4(CCCCC4)NC5=O)c23)C1]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 105 μL N-ethyl-N-isopropylpropan-2-amine, 250 μL N-methylmethanamine (2M in tetrahydrofuran) and 318 μL 2,4,6-tripropyl-1,3,5,2,4,6-trioxatriphosphinane 2,4,6-trioxide solution (50% in N,N-dimethylformamide) was stirred at RT overnight. Water was added, the precipitate was washed with water, ethanol and diethylether and dried to give 42 mg (76%) of the title compound.

LC-MS: m/z=527.1 [M+H]⁺.

¹H-NMR (400 MHz, DMSO-d6), δ [ppm]=1.27 (1H), 1.57 (2H), 1.66 (3H), 1.73-1.83 (3H), 2.17 (1H), 2.85-3.06 (4H), 2.88 (3H), 3.11 (3H), 3.16-3.27 (3H), 8.68 (1H), 8.86 (1H), 8.96 (1H), 10.40 (1H).

### Example 76

### 6′-{[(7S)-7-(Azetidin-1-ylcarbonyl)-5,6,7,8-tetrahydro[1]benzothieno[2,3-d]pyrimidin-4-yl]amino}-8′-chloro-2′H-spiro[cyclohexane-1,3′-imidazo[1,5-a]pyridine]-1′,5′-dione

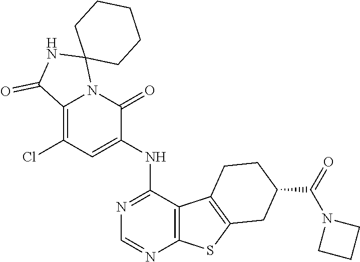 [SMILES: O=C1NC2(CCCCC2)n2c1c(Cl)cc(Nc1ncnc3sc4c(c13)CC[C@H](C(=O)N1CCC1)C4)c2=O]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 105 μL N-ethyl-N-isopropylpropan-2-amine, 22.8 mg azetidine and 179 μL 2,4,6-tripropyl-1,3,5,2,4,6-trioxatriphosphinane 2,4,6-trioxide solution (50% in N,N-dimethylformamide) was stirred at RT overnight. Water was added, the precipitate was washed with water, ethanol and diethylether and dried to give 48 mg (84%) of the title compound.

LC-MS: m/z=539.1 [M+H]⁺.

¹H-NMR (400 MHz, DMSO-d6), δ [ppm]=1.27 (1H), 1.57 (2H), 1.61-1.73 (3H), 1.73-1.86 (3H), 2.15 (1H), 2.22 (2H), 2.76 (1H), 2.87-2.98 (4H), 3.11-3.28 (2H), 3.89 (2H), 4.28 (2H), 8.68 (1H), 8.85 (1H), 8.95 (1H), 10.36 (1H).

### Example 77

### 1-({(7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile

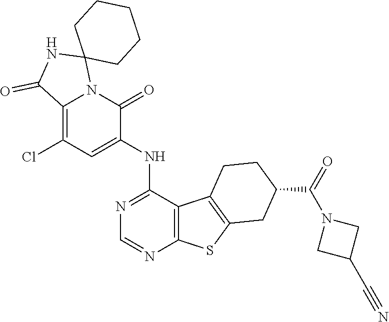 [SMILES: N#CC1CN(C(=O)[C@H]2CCc3c(sc4ncnc(Nc5cc(Cl)c6n(c5=O)C5(CCCCC5)NC6=O)c34)C2)C1]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 157 μL N-ethyl-N-isopropylpropan-2-amine, 59 mg azetidine-3-carbonitrile and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 53 mg (90%) of the title compound.

LC-MS: m/z=564.1 [M+H]⁺.

¹H-NMR (400 MHz, DMSO-d6), δ [ppm]=1.27 (1H), 1.57 (2H), 1.61-1.73 (3H), 1.73-1.87 (3H), 2.17 (1H), 2.77 (1H), 2.86-3.03 (4H), 3.13-3.29 (2H), 3.81 (1H), 4.06 (1H), 4.19 (1H), 4.54 (2H), 8.68 (1H), 8.85 (1H), 8.96 (1H), 10.40 (1H).

### Example 78

### 8′-Chloro-6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione

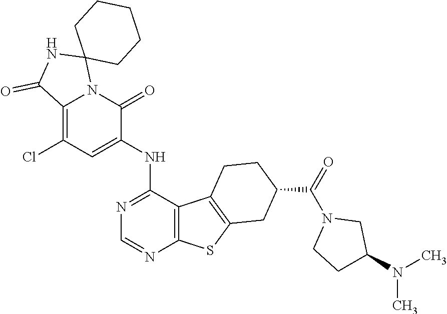 [SMILES: CN(C)[C@H]1CCN(C(=O)[C@H]2CCc3c(sc4ncnc(Nc5cc(Cl)c6n(c5=O)C5(CCCCC5)NC6=O)c34)C2)C1]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 105 μL N-ethyl-N-isopropylpropan-2-amine, 57 mg (3S)—N,N-dimethylpyrrolidin-3-amine and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 48 mg (76%) of the title compound.

LC-MS: m/z=596.2 [M+H]⁺.

¹H-NMR (400 MHz, DMSO-d6), δ [ppm]=1.27 (1H), 1.57 (2H), 1.61-1.73 (3H), 1.78 (3H), 1.99-2.13 (1H), 2.17 (3H), 2.18 (3H), 2.61+2.73 (1H), 2.85-3.07 (6H), 3.14-3.66 (6H*), 3.78+3.87 (1H), 8.68 (1H), 8.86+8.87 (1H), 8.97+8.98 (1H), 10.40 (1H);

*: at least partially hidden by solvent peak.

### Example 79

### 8′-Chloro-6′-{[(7S)-7-{[(3R)-3-methylmorpholin-4-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione

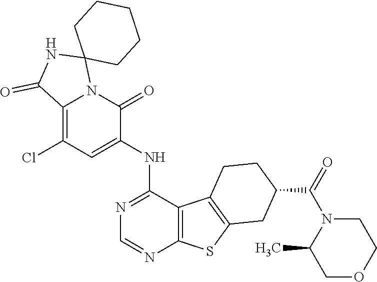 [SMILES: C[C@@H]1COCCN1C(=O)[C@H]1CCc2c(sc3ncnc(Nc4cc(Cl)c5n(c4=O)C4(CCCCC4)NC5=O)c23)C1]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 105 μL N-ethyl-N-isopropylpropan-2-amine, 51 mg (3R)-3-methylmorpholine and 179 μL 2,4,6-tripropyl-1,3,5,2,4,6-trioxatriphosphinane 2,4,6-trioxide solution (50% in N,N-dimethylformamide) was stirred at 50° C. overnight. 51 mg (3R)-3-methylmorpholine and 179 μL 2,4,6-tripropyl-1,3,5,2,4,6-trioxatriphosphinane 2,4,6-trioxide solution (50% in N,N-dimethylformamide) were added and stirring continued for 24 hours.

Water was added, the precipitate was washed with water, ethanol and diethylether and dried to give 48 mg (79%) of the title compound.

LC-MS: m/z=583.2 [M+H]⁺.

¹H-NMR (400 MHz, DMSO-d6), δ [ppm]=1.15 (2H), 1.21-1.38 (3H), 1.57 (2H), 1.66 (3H), 1.77 (2H), 1.82-1.99 (1H), 2.14 (1H), 2.86-3.05 (4H), 3.11 (1H), 3.24 (2H), 3.38-3.82 (3H), 3.85 (1H), 4.12+4.19 (1H), 4.42 (1H), 8.68 (1H), 8.86 (1H), 8.96 (1H), 10.40 (1H).

### Example 80

### 8′-Chloro-6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione

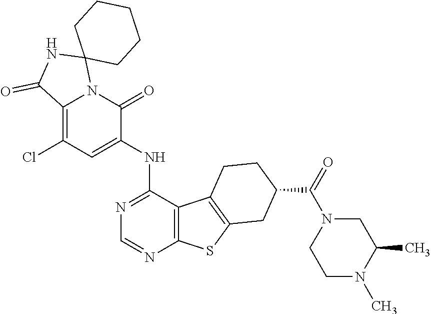 [SMILES: C[C@@H]1CN(C(=O)[C@H]2CCc3c(sc4ncnc(Nc5cc(Cl)c6n(c5=O)C5(CCCCC5)NC6=O)c34)C2)CCN1C]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 261 μL N-ethyl-N-isopropylpropan-2-amine, 291 mg (2R)-1,2-dimethylpiperazine dihydrochloride and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 58 mg (92%) of the title compound.

LC-MS: m/z=596.2 [M+H]⁺.

¹H-NMR (400 MHz, DMSO-d6), δ [ppm]=0.94-1.08 (6H), 1.23+1.48 (3H), 1.27 (1H), 1.41-1.52 (3H), 1.57 (2H), 1.61-1.72 (3H), 1.78 (2H), 1.94-2.26 (4H), 2.78 (1H), 2.86-3.11 (3H), 3.92 (1H), 4.17 (1H), 8.70 (1H), 8.88 (1H), 8.99 (1H), 10.41 (1H).

### Example 81

### 8′-Chloro-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione

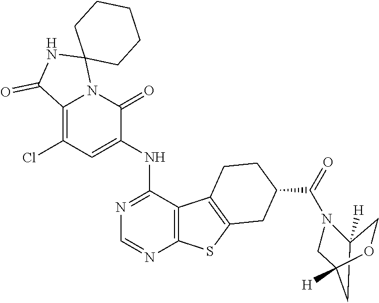 [SMILES: O=C1NC2(CCCCC2)n2c1c(Cl)cc(Nc1ncnc3sc4c(c13)CC[C@H](C(=O)N1C[C@H]3C[C@@H]1CO3)C4)c2=O]

A mixture of 50 mg (100 μmol) (7S)-4-[(8′-Chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid (prepared according to example 73), 3.0 mL N,N-dimethylacetamide, 157 μL N-ethyl-N-isopropylpropan-2-amine, 68 mg (1R,4R)-2-oxa-5-azabicyclo[2.2.1]heptane hydrochloride and 179 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 54 mg (88%) of the title compound.

LC-MS: m/z=581.0 [M+H]⁺.

¹H-NMR (400 MHz, DMSO-d6), δ [ppm]=1.27 (1H), 1.57 (2H), 1.61-1.73 (3H), 1.73-1.93 (5H), 2.16+2.23 (1H), 2.77-3.13 (5H), 3.16-3.39 (3H*), 3.52-3.61 (1H), 3.64-3.80 (2H), 4.62+4.67 (1H), 4.78+4.90 (1H), 8.68 (1H), 8.84 (1H), 8.98 (1H), 10.40 (1H);

*: at least partially hidden by solvent peak.

### Example 82

### 1-{[(7S)-4-{[(cis)-4-Hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidin-7-yl]carbonyl}azetidine-3-carbonitrile

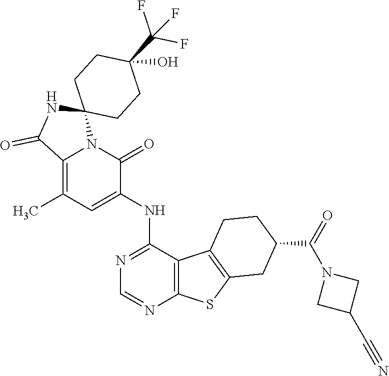 [SMILES: Cc1cc(Nc2ncnc3sc4c(c23)CC[C@H](C(=O)N2CC(C#N)C2)C4)c(=O)n2c1C(=O)N[C@]21CC[C@@](O)(C(F)(F)F)CC1]

A mixture of 59 mg (105 μmol) (7S)-4-{[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 3.5 mL N,N-dimethylacetamide, 164 μL N-ethyl-N-isopropylpropan-2-amine, 62 mg azetidine-3-carbonitrile and 187 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 49 mg (71%) of the title compound.

LC-MS: m/z=628.5 [M+H]⁺.

¹H-NMR (400 MHz, DMSO-d6), δ [ppm]=1.44 (2H), 1.75-1.89 (3H), 1.96 (2H), 2.16 (1H), 2.50 (3H*), 2.78 (1H), 2.85-3.04 (2H), 3.13-3.50 (4H*), 3.81 (1H), 4.05 (1H), 4.19 (1H), 4.45-4.61 (2H), 6.03 (1H), 8.64 (1H), 8.75 (1H), 9.01 (1H), 10.42 (1H);

*: at least partially hidden by solvent peak.

### Example 83

### (cis)-6′-{[(7S)-7-{[(3R)-3,4-Dimethylpiperazin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione

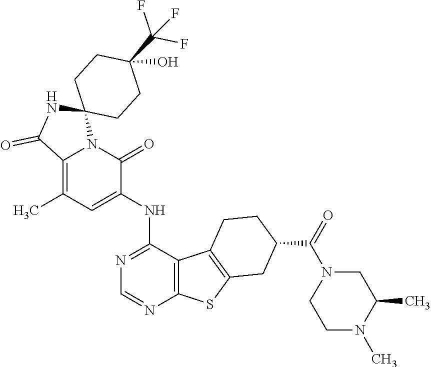 [SMILES: Cc1cc(Nc2ncnc3sc4c(c23)CC[C@H](C(=O)N2CCN(C)[C@H](C)C2)C4)c(=O)n2c1C(=O)N[C@]21CC[C@@](O)(C(F)(F)F)CC1]

A mixture of 59 mg (105 μmol) (7S)-4-{[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidine-7-carboxylic acid (prepared according to example 59), 3.5 mL N,N-dimethylacetamide, 274 μL N-ethyl-N-isopropylpropan-2-amine, 98 mg (2R)-1,2-dimethylpiperazine dihydrochloride and 187 μL 2,4,6-tripropyl-1,3,5,2,4,6-trioxatriphosphinane 2,4,6-trioxide solution (50% in N,N-dimethylformamide) was stirred at 50° C. overnight. 274 μL N-ethyl-N-isopropylpropan-2-amine and 98 mg (2R)-1,2-dimethylpiperazine dihydrochloride were added and stirring continued for 24 hours. Water was added, the precipitate was washed with water, ethanol and diethylether and dried to give 39 mg (54%) of the title compound.

LC-MS: m/z=660.6 [M+H]⁺.

¹H-NMR (400 MHz, DMSO-d6), δ [ppm]=1.02 (3H), 1.38-1.52 (3H), 1.75-2.16 (8H), 2.20 (3H), 2.50 (3H*), 2.71-3.52 (9H*), 3.93 (1H), 4.16 (1H), 6.04 (1H), 8.64 (1H), 8.76 (1H), 9.02 (1H), 10.42 (1H);

*: at least partially hidden by solvent peak.

### Example 84

### (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid

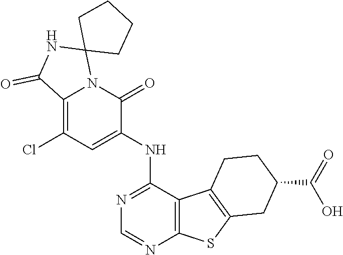 [SMILES: O=C1NC2(CCCC2)n2c1c(Cl)cc(Nc1ncnc3sc4c(c13)CC[C@H](C(=O)O)C4)c2=O]

A mixture of 703 mg (1.37 mmol) ethyl (7S)-4-[(8′-chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylate (prepared according to example 74a) in 20 mL tetrahydrofuran, 10 mL ethanol and 8.21 mL lithium hydroxide solution (1M in water) was stirred at RT for 4 hours. Water was added and the mixture was acidified with hydrochloric acid. After concentration the precipitate was washed with water and dried to give 635 mg (91%) of the title compound.

LC-MS: m/z=486.1 [M+H]⁺.

¹H-NMR (400 MHz, DMSO-d6), δ [ppm]=1.69-1.79 (2H), 1.79-1.90 (2H), 1.93-2.05 (3H), 2.25 (1H), 2.78-2.94 (3H), 3.01 (1H), 3.10 (1H), 3.19 (2H), 8.68 (1H), 8.84 (1H), 8.92 (1H), 10.20 (1H), 12.52 (1H).

### Example 84a

### Ethyl (7S)-4-[(8′-chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylate

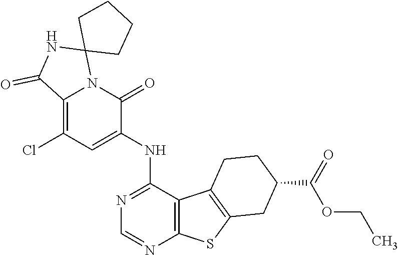 [SMILES: CCOC(=O)[C@H]1CCc2c(sc3ncnc(Nc4cc(Cl)c5n(c4=O)C4(CCCC4)NC5=O)c23)C1]

To a solution of 500 mg (1.80 mmol) ethyl (7S)-4-amino-5,6,7,8-tetrahydro[1]benzothieno[2,3-d]pyrimidine-7-carboxylate (prepared according to example 1 b) and 630 mg (1.98 mmol) 6′-bromo-8′-chloro-2′H-spiro[cyclopentane-1,3′-imidazo[1,5-a]pyridine]-1′,5′-dione (prepared according to example 84b) in 70 mL 1,4-dioxane were added 1.76 g cesium carbonate and the mixture was degassed and purged with argon several times. 112 mg 4,5-bis(diphenylphosphino)-9,9-dimethylxanthene, 92 mg 2-(dicyclohexyl-phosphino)-2′,4′,6′-triisopropylbiphenyl, 43.3 mg palladium(II)acetate and 177 mg tris(dibenzylideneacetone)dipalladium(0) were added and the mixture was stirred at 100° C. for 2 hours. After concentration the residue was purified by flash chromatography (Biotage SNAP cartridge silica 100 g, ethanol:dichloromethane) and crystallization from ethanol to give 705 mg (76%) of the title compound.

LC-MS: m/z=514.4 [M+H]⁺.

### Example 84b

### 6′-Bromo-8′-chloro-2′H-spiro[cyclopentane-1,3′-imidazo[1,5-a]pyridine]-1′,5′-dione

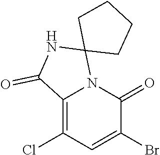 [SMILES: O=C1NC2(CCCC2)n2c1c(Cl)cc(Br)c2=O]

To a solution of 3.00 g (11.9 mmol) 5-bromo-3-chloro-6-oxo-1,6-dihydropyridine-2-carboxamide (PCT Int. Appl. (2015), WO 2015200481) and 2.01 g (23.9 mmol) cyclopentanone in 35 mL 1,4-dioxane were added 314 μl sulfuric acid and the mixture was stirred at 95° C. for 3 hours. After concentration water was added, the precipitate washed with water and diethyl ether and dried to give 3.41 g (90%) of the title compound.

LC-MS: m/z=3.17.0 [M+H]⁺.

### Example 85

### (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxamide

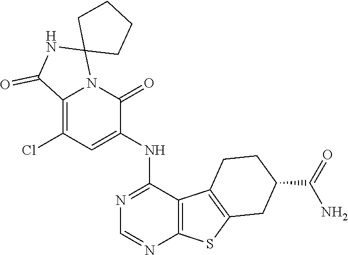 [SMILES: NC(=O)[C@H]1CCc2c(sc3ncnc(Nc4cc(Cl)c5n(c4=O)C4(CCCC4)NC5=O)c23)C1]

A mixture of 80 mg (165 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 5.0 mL N,N-dimethylacetamide, 172 μL N-ethyl-N-isopropylpropan-2-amine, 4.1 mL ammonia (0.4M in tetrahydrofurane) and 294 μL 2,4,6-tripropyl-1,3,5,2,4,6-trioxatriphosphinane 2,4,6-trioxide solution (50% in N,N-dimethylformamide) was stirred at 60° C. overnight. 4.1 mL ammonia (0.4M in tetrahydrofurane) and 294 μL 2,4,6-tripropyl-1,3,5,2,4,6-trioxatriphosphinane 2,4,6-trioxide solution (50% in N,N-dimethylformamide) were added and stirring continued for 2.5 days. Water was added, the precipitate was washed with water and ethanol and dried to give 55 mg (65%) of the title compound.

LC-MS: m/z=485.4 [M+H]⁺.

¹H-NMR (400 MHz, DMSO-d6), δ [ppm]=1.70-1.78 (2H), 1.79-2.03 (5H), 2.20 (1H), 2.668 (1H), 2.78-2.89 (2H), 2.90-3.03 (2H), 3.13 (1H), 3.24 (1H), 6.99 (1H), 7.47 (1H), 8.68 (1H), 8.84 (1H), 8.94 (1H), 10.20 (1H).

### Example 86

### (7S)-4-[(8′-Chloro-1′,5′-dioxo-1′,5′-dihydro-2′H-spiro[cyclopentane-1,3′-imidazo[1,5-a]pyridin]-6′-yl)amino]-N,N-dimethyl-5,6,7,8-tetrahydro[1]benzothieno[2,3-d]pyrimidine-7-carboxamide

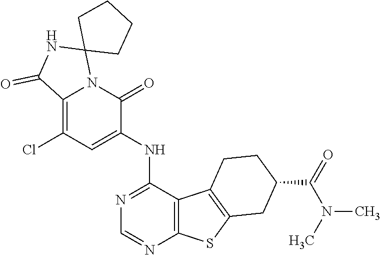 [SMILES: CN(C)C(=O)[C@H]1CCc2c(sc3ncnc(Nc4cc(Cl)c5n(c4=O)C4(CCCC4)NC5=O)c23)C1]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 108 μL N-ethyl-N-isopropylpropan-2-amine, 257 μL N-methylmethanamine (2M in tetrahydrofuran) and 184 μL 2,4,6-tripropyl-1,3,5,2,4,6-trioxatriphosphinane 2,4,6-trioxide solution (50% in N,N-dimethylformamide) was stirred at 50° C. overnight. After concentration, water was added, the precipitate was washed with water, ethanol and diethylether and dried to give 46 mg (83%) of the title compound.

LC-MS: m/z=513.4 [M+H]⁺.

¹H-NMR (400 MHz, DMSO-d6), δ [ppm]=1.70-1.90 (5H), 1.92-2.03 (2H), 2.16 (1H), 2.78-2.92 (2H), 2.88 (3H), 2.92-3.05 (2H), 3.11 (3H), 3.15-3.28 (3H), 8.69 (1H), 8.87 (1H), 8.93 (1H), 10.20 (1H).

### Example 87

### 6′-{[(7S)-7-(Azetidine-1-carbonyl)-5,6,7,8-tetrahydro[1]benzothieno[2,3-d]pyrimidin-4-yl]amino}-8′-chloro-2′H-spiro[cyclopentane-1,3′-imidazo[1,5-a]pyridine]-1′,5′-dione

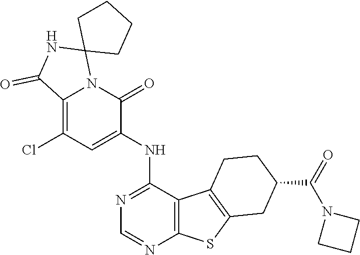 [SMILES: O=C1NC2(CCCC2)n2c1c(Cl)cc(Nc1ncnc3sc4c(c13)CC[C@H](C(=O)N1CCC1)C4)c2=O]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 108 μL N-ethyl-N-isopropylpropan-2-amine, 24 mg azetidine and 184 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 49 mg (87%) of the title compound.

LC-MS: m/z=525.4 [M+H]⁺.

¹H-NMR (400 MHz, DMSO-d6), δ [ppm]=1.68-1.91 (5H), 1.92-2.03 (2H), 2.14 (1H), 2.22 (2H), 2.72-2.98 (5H), 3.10-3.30 (2H), 3.89 (2H), 4.26 (2H), 8.69 (1H), 8.87 (1H), 8.93 (1H), 10.20 (1H).

### Example 88

### 1-{(7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carbonyl}azetidine-3-carbonitrile

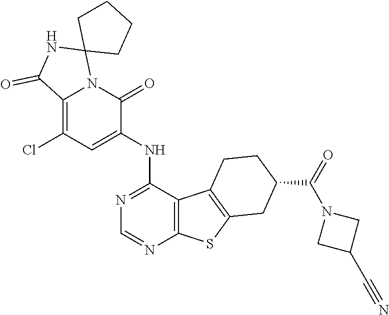 [SMILES: N#CC1CN(C(=O)[C@H]2CCc3c(sc4ncnc(Nc5cc(Cl)c6n(c5=O)C5(CCCC5)NC6=O)c34)C2)C1]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 161 μL N-ethyl-N-isopropylpropan-2-amine, 61 mg azetidine-3-carbonitrile and 184 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 42 mg (70%) of the title compound.

LC-MS: m/z=550.1 [M+H]⁺.

¹H-NMR (400 MHz, DMSO-d6), δ [ppm]=1.46 (1H), 1.67-2.03 (7H), 2.16 (1H), 2.72-3.25 (6H), 3.81 (1H), 4.06 (1H), 4.19 (1H), 4.54 (2H), 8.70 (1H), 8.88 (1H), 8.96 (1H), 10.21 (1H).

### Example 89

### 8′-Chloro-6′-({(7S)-7-[(3S)-3-(dimethylamino)pyrrolidine-1-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione

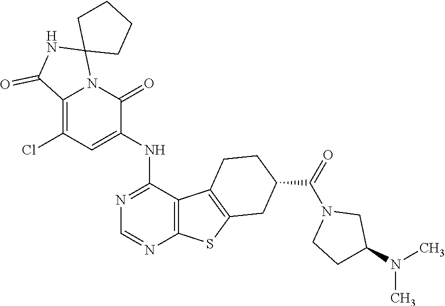 [SMILES: CN(C)[C@H]1CCN(C(=O)[C@H]2CCc3c(sc4ncnc(Nc5cc(Cl)c6n(c5=O)C5(CCCC5)NC6=O)c34)C2)C1]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 108 μL N-ethyl-N-isopropylpropan-2-amine, 59 mg (3S)—N,N-dimethylpyrrolidin-3-amine and 184 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 47 mg (75%) of the title compound.

LC-MS: m/z=582.5 [M+H]⁺.

¹H-NMR (400 MHz, DMSO-d6), δ [ppm]=1.55-1.89 (6H), 1.92-2.24 (4H), 2.17 (6H), 2.61+2.73 (1H), 2.77-2.88 (2H), 2.92-3.04 (3H), 3.18-3.30 (3H), 3.41-3.66 (2H), 3.78+3.87 (1H), 8.70 (1H), 8.89 (1H), 8.96 (1H), 10.20 (1H).

### Example 90

### 8′-Chloro-6′-({(7S)-7-[(3R)-3-methylmorpholine-4-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione

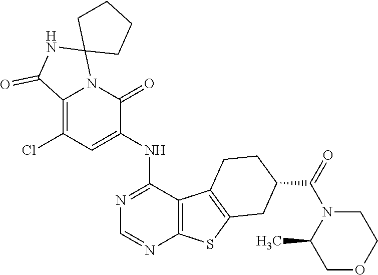 [SMILES: C[C@@H]1COCCN1C(=O)[C@H]1CCc2c(sc3ncnc(Nc4cc(Cl)c5n(c4=O)C4(CCCC4)NC5=O)c23)C1]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 108 μL N-ethyl-N-isopropylpropan-2-amine, 52 mg (3R)-3-methylmorpholine and 184 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 54 mg (87%) of the title compound.

LC-MS: m/z=569.4 [M+H]⁺.

¹H-NMR (400 MHz, DMSO-d6), δ [ppm]=1.15+1.33 (3H), 1.68-2.03 (7H), 2.13 (1H), 2.83 (2H), 2.90-3.05 (2H), 3.11 (1H), 3.18-3.57 (5H*), 3.63 (1H), 3.71-4.47 (3H), 8.69 (1H), 8.87 (1H), 8.93 (1H), 10.22 (1H);

*: at least partially hidden by solvent peak.

### Example 91

### 8′-Chloro-6′-({(7S)-7-[(3R)-3,4-dimethylpiperazine-1-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione

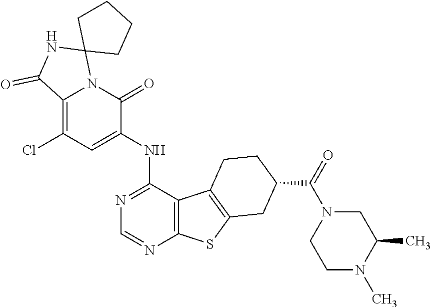 [SMILES: C[C@@H]1CN(C(=O)[C@H]2CCc3c(sc4ncnc(Nc5cc(Cl)c6n(c5=O)C5(CCCC5)NC6=O)c34)C2)CCN1C]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.0 mL N,N-dimethylacetamide, 269 μL N-ethyl-N-isopropylpropan-2-amine, 96 mg (2R)-1,2-dimethylpiperazine dihydrochloride and 184 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 51 mg (81%) of the title compound.

LC-MS: m/z=582.5 [M+H]⁺.

¹H-NMR (400 MHz, DMSO-d6), δ [ppm]=0.99+1.03 (3H), 1.69-2.16 (10H), 2.19 (3H), 2.70-2.95 (5H), 3.00 (1H), 3.16-3.29 (4H), 3.91 (1H), 4.16 (1H), 8.69 (1H), 8.87 (1H), 8.93 (1H), 10.21 (1H).

### Example 92

### 8′-Chloro-6′-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione

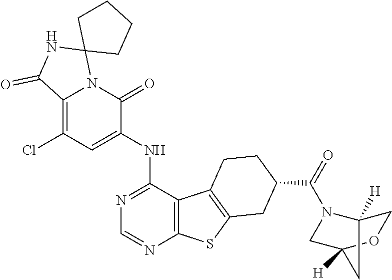 [SMILES: O=C1NC2(CCCC2)n2c1c(Cl)cc(Nc1ncnc3sc4c(c13)CC[C@H](C(=O)N1C[C@H]3C[C@@H]1CO3)C4)c2=O]

A mixture of 50 mg (103 μmol) (7S)-4-[(8′-Chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid (prepared according to example 84), 3.1 mL N,N-dimethylacetamide, 129 μL N-ethyl-N-isopropylpropan-2-amine, 70 mg (1R,4R)-2-oxa-5-azabicyclo[2.2.1]heptane hydrochloride and 318 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 53 mg (87%) of the title compound.

LC-MS: m/z=567.4 [M+H]⁺.

¹H-NMR (400 MHz, DMSO-d6), δ [ppm]=1.69-2.04 (9H), 2.15+2.23 (1H), 2.77-2.89 (2H), 2.90-3.12 (3H), 3.18-3.30 (3H), 3.56 (1H), 3.63-3.78 (2H), 4.61+4.66 (1H), 4.78+4.91 (1H), 8.69 (1H), 8.86 (1H), 8.96 (1H), 10.21 (1H).

### Example 93

### (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid

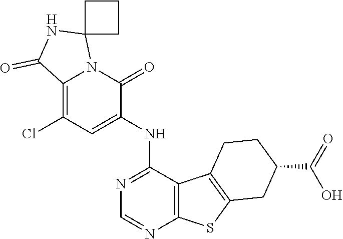 [SMILES: O=C1NC2(CCC2)n2c1c(Cl)cc(Nc1ncnc3sc4c(c13)CC[C@H](C(=O)O)C4)c2=O]

A mixture of 637 mg (1.27 mmol) ethyl (7S)-4-[(8′-chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylate (prepared according to example 93a) in 20 mL tetrahydrofuran, 10 mL ethanol and 7.6 mL lithium hydroxide solution (1M in water) was stirred at RT for 4 hours. Water was added and the mixture was acidified with hydrochloric acid. After concentration the precipitate was washed with water and dried to give 603 mg (95%) of the title compound.

LC-MS: m/z=472.1 [M+H]⁺.

¹H-NMR (400 MHz, DMSO-d6), δ [ppm]=1.91-2.08 (2H), 2.13 (1H), 2.22-2.39 (3H), 2.90 (1H), 3.01 (1H), 3.11 (1H), 3.16-3.27 (2H), 3.46 (2H), 8.68 (1H), 8.82 (1H), 8.94 (1H), 10.37 (1H), 12.52 (1H).

### Example 93a

### Ethyl (7S)-4-[(8′-chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylate

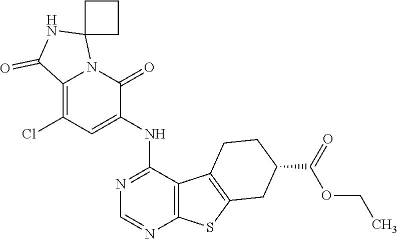 [SMILES: CCOC(=O)[C@H]1CCc2c(sc3ncnc(Nc4cc(Cl)c5n(c4=O)C4(CCC4)NC5=O)c23)C1]

To a solution of 500 mg (1.80 mmol) ethyl (7S)-4-amino-5,6,7,8-tetrahydro[1]benzothieno[2,3-d]pyrimidine-7-carboxylate (prepared according to example 1 b) and 602 mg (1.98 mmol) 6′-bromo-8′-chloro-2′H-spiro[cyclobutane-1,3′-imidazo[1,5-a]pyridine]-1′,5′-dione (prepared according to example 93b) in 70 mL 1,4-dioxane were added 1.76 g cesium carbonate and the mixture was degassed and purged with argon several times. 112 mg 4,5-bis(diphenylphosphino)-9,9-dimethylxanthene, 92 mg 2-(dicyclohexyl-phosphino)-2′,4′,6′-triisopropylbiphenyl, 43 mg palladium(II)acetate and 177 mg tris(dibenzylideneacetone)dipalladium(0) were added and the mixture was stirred at 100° C. for 2 hours. After concentration the residue was purified by flash chromatography (Biotage SNAP cartridge silica 100 g, ethanol:dichloromethane) and crystallization from ethanol to give 637 mg (71%) of the title compound.

LC-MS: m/z=500.1 [M+H]⁺.

### Example 93b

### 6′-Bromo-8′-chloro-2′H-spiro[cyclobutane-1,3′-imidazo[1,5-a]pyridine]-1′,5′-dione

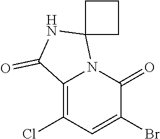 [SMILES: O=C1NC2(CCC2)n2c1c(Cl)cc(Br)c2=O]

To a solution of 3.00 g (11.9 mmol) 5-bromo-3-chloro-6-oxo-1,6-dihydropyridine-2-carboxamide (PCT Int. Appl. (2015), WO 2015200481) and 1.67 g (23.9 mmol) cyclobutanone in 35 mL 1,4-dioxane were added 314 μl sulfuric acid and the mixture was stirred at 95° C. for 3 hours. After concentration water was added, the precipitate washed with water and diethyl ether and dried to give 2.36 g (65%) of the title compound.

LC-MS: m/z=303.9 [M+H]⁺.

### Example 94

### (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxamide

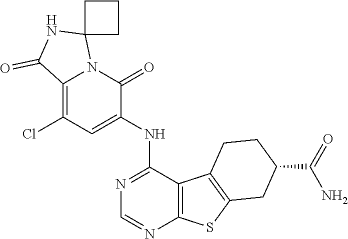 [SMILES: NC(=O)[C@H]1CCc2c(sc3ncnc(Nc4cc(Cl)c5n(c4=O)C4(CCC4)NC5=O)c23)C1]

A mixture of 80 mg (170 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 5.2 mL N,N-dimethylacetamide, 177 μL N-ethyl-N-isopropylpropan-2-amine, 4.2 mL ammonia (0.4M in tetrahydrofurane) and 303 μL 2,4,6-tripropyl-1,3,5,2,4,6-trioxatriphosphinane 2,4,6-trioxide solution (50% in N,N-dimethylformamide) was stirred at 60° C. overnight. 4.2 mL ammonia (0.4M in tetrahydrofurane) and 303 μL 2,4,6-tripropyl-1,3,5,2,4,6-trioxatriphosphinane 2,4,6-trioxide solution (50% in N,N-dimethylformamide) were added and stirring continued for 2.5 days. Water was added, the precipitate was washed with water and ethanol and dried to give 47 mg (56%) of the title compound.

LC-MS: m/z=471.3 [M+H]⁺.

¹H-NMR (400 MHz, DMSO-d6), δ [ppm]=1.93 (2H), 2.14 (1H), 2.23 (1H), 2.29-2.40 (2H), 2.69 (1H), 2.91-3.04 (2H), 3.16 (1H), 3.27 (1H), 3.45 (2H), 7.00 (1H), 7.48 (1H), 8.69 (1H), 8.83 (1H), 8.97 (1H), 10.37 (1H).

### Example 95

### (7S)-4-[(8′-Chloro-1′,5′-dioxo-1′,5′-dihydro-2′H-spiro[cyclobutane-1,3′-imidazo[1,5-a]pyridin]-6′-yl)amino]-N,N-dimethyl-5,6,7,8-tetrahydro[1]benzothieno[2,3-d]pyrimidine-7-carboxamide

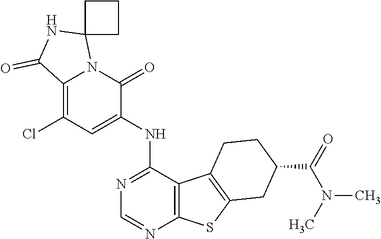 [SMILES: CN(C)C(=O)[C@H]1CCc2c(sc3ncnc(Nc4cc(Cl)c5n(c4=O)C4(CCC4)NC5=O)c23)C1]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 111 μL N-ethyl-N-isopropylpropan-2-amine, 265 μL N-methylmethanamine (2M in tetrahydrofuran) and 189 μL 2,4,6-tripropyl-1,3,5,2,4,6-trioxatriphosphinane 2,4,6-trioxide solution (50% in N,N-dimethylformamide) was stirred at 50° C. overnight. After concentration water was added, the precipitate was washed with water, ethanol and diethylether and dried to give 45 mg (82%) of the title compound.

LC-MS: m/z=499.1 [M+H]⁺.

¹H-NMR (400 MHz, DMSO-d6), δ [ppm]=1.82 (1H), 1.95 (1H), 2.07-2.22 (2H), 2.33 (2H), 2.88 (3H), 2.89-3.06 (2H), 3.12 (3H), 3.16-3.30 (3H), 3.45 (2H), 8.69 (1H), 8.85 (1H), 8.96 (1H), 10.37 (1H).

### Example 96

### 6′-{[(7S)-7-(Azetidine-1-carbonyl)-5,6,7,8-tetrahydro[1]benzothieno[2,3-d]pyrimidin-4-yl]amino}-8′-chloro-2′H-spiro[cyclobutane-1,3′-imidazo[1,5-a]pyridine]-1′,5′-dione

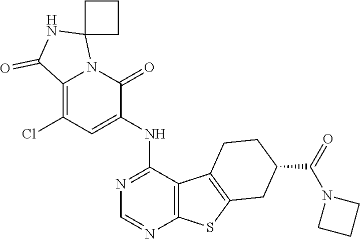 [SMILES: O=C1NC2(CCC2)n2c1c(Cl)cc(Nc1ncnc3sc4c(c13)CC[C@H](C(=O)N1CCC1)C4)c2=O]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 111 μL N-ethyl-N-isopropylpropan-2-amine, 24 mg azetidine and 189 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 47 mg (82%) of the title compound.

LC-MS: m/z=511.1 [M+H]⁺.

¹H-NMR (400 MHz, DMSO-d6), δ [ppm]=1.83 (1H), 1.95 (1H), 2.07-2.26 (4H), 2.33 (2H), 2.78 (1H), 2.93 (2H), 3.12-3.30 (2H), 3.45 (2H), 3.89 (2H), 4.27 (2H), 8.68 (1H), 8.84 (1H), 8.95 (1H), 10.37 (1H).

### Example 97

### 1-{(7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carbonyl}azetidine-3-carbonitrile

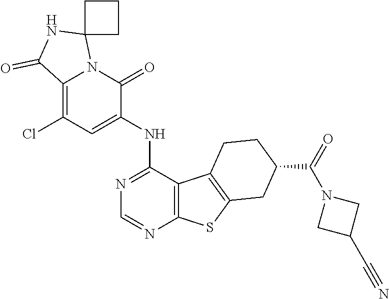 [SMILES: N#CC1CN(C(=O)[C@H]2CCc3c(sc4ncnc(Nc5cc(Cl)c6n(c5=O)C5(CCC5)NC6=O)c34)C2)C1]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 166 μL N-ethyl-N-isopropylpropan-2-amine, 63 mg azetidine-3-carbonitrile and 189 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 37 mg (61%) of the title compound.

LC-MS: m/z=536.4 [M+H]⁺.

¹H-NMR (400 MHz, DMSO-d6), δ [ppm]=1.82 (1H), 1.96 (1H), 2.07-2.26 (2H), 2.34 (2H), 2.79 (1H), 2.86-3.04 (2H), 3.25 (2H), 3.45 (2H), 3.82 (1H), 4.06 (1H), 4.19 (1H), 4.55 (2H), 8.69 (1H), 8.85 (1H), 8.97 (1H), 10.37 (1H).

### Example 98

### 8′-Chloro-6′-({(7S)-7-[(3S)-3-(dimethylamino)pyrrolidine-1-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione

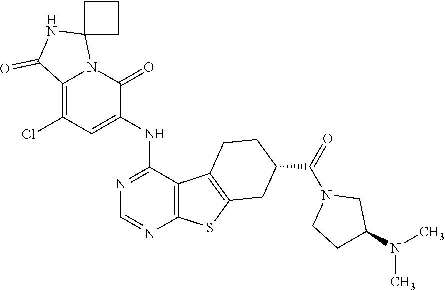 [SMILES: CN(C)[C@H]1CCN(C(=O)[C@H]2CCc3c(sc4ncnc(Nc5cc(Cl)c6n(c5=O)C5(CCC5)NC6=O)c34)C2)C1]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 111 μL N-ethyl-N-isopropylpropan-2-amine, 60 mg (3S)—N,N-dimethylpyrrolidin-3-amine and 189 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 51 mg (80%) of the title compound.

LC-MS: m/z=568.2 [M+H]⁺.

¹H-NMR (400 MHz, DMSO-d6), δ [ppm]=1.57-2.26 (6H), 2.17 (3H), 2.18 (3H), 2.33 (2H), 2.62+2.74 (1H), 2.91-3.08 (3H), 3.19-3.68 (7H*), 3.80+3.88 (1H), 8.69 (1H), 8.86 (1H), 8.97 (1H), 10.37 (1H);

*: at least partially hidden by solvent peak.

### Example 99

### 8′-Chloro-6′-({(7S)-7-[(3R)-3-methylmorpholine-4-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione

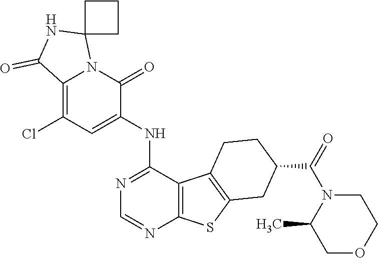 [SMILES: C[C@@H]1COCCN1C(=O)[C@H]1CCc2c(sc3ncnc(Nc4cc(Cl)c5n(c4=O)C4(CCC4)NC5=O)c23)C1]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 111 μL N-ethyl-N-isopropylpropan-2-amine, 60 μL (3R)-3-methylmorpholine and 189 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 41 mg (65%) of the title compound.

LC-MS: m/z=555.4[M+H]⁺.

¹H-NMR (400 MHz, DMSO-d6), δ [ppm]=1H-NMR (400 MHz, DMSO-d6), Shift [ppm]=1.15+1.34 (3H), 1.85-2.01 (2H), 2.06-2.21 (2H), 2.33 (2H), 2.86-3.59 (10H*), 3.64 (1H), 3.74-4.47 (3H), 8.69 (1H), 8.86 (1H), 8.96 (1H), 10.38 (1H);

*: at least partially hidden by solvent peak.

### Example 100

### 8′-Chloro-6′-({(7S)-7-[(3R)-3,4-dimethylpiperazine-1-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione

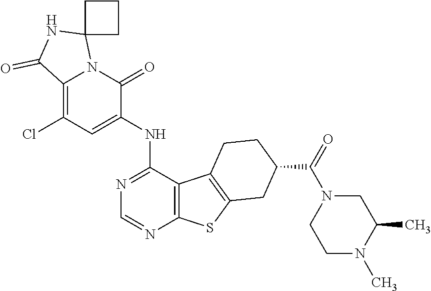 [SMILES: C[C@@H]1CN(C(=O)[C@H]2CCc3c(sc4ncnc(Nc5cc(Cl)c6n(c5=O)C5(CCC5)NC6=O)c34)C2)CCN1C]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 277 μL N-ethyl-N-isopropylpropan-2-amine, 99 mg (2R)-1,2-dimethylpiperazine dihydrochloride and 189 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 51 mg (80%) of the title compound.

LC-MS: m/z=568.5 [M+H]⁺.

¹H-NMR (400 MHz, DMSO-d6), δ [ppm]=1.00+1.04 (3H), 1.77-2.04 (3H), 2.13 (2H), 2.20 (3H), 2.33 (2H), 2.70-3.11 (4H), 3.16-3.53 (7H*), 3.92 (1H), 4.17 (1H), 8.69 (1H), 8.86 (1H), 8.96 (1H), 10.38 (1H);

*: at least partially hidden by solvent peak.

### Example 101

### (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid

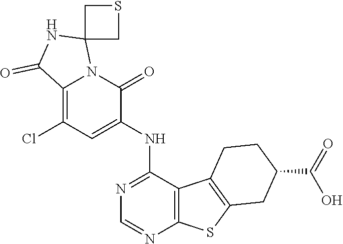 [SMILES: O=C1NC2(CSC2)n2c1c(Cl)cc(Nc1ncnc3sc4c(c13)CC[C@H](C(=O)O)C4)c2=O]

A mixture of 683 mg (1.32 mmol) ethyl (7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate (prepared according to example 101a) in 20 mL tetrahydrofuran, 10 mL ethanol and 7.9 mL lithium hydroxide solution (1M in water) was stirred at RT for 4 hours. Water was added and the mixture was acidified with hydrochloric acid. After concentration the precipitate was washed with water and dried to give 665 mg (98%) of the title compound.

LC-MS: m/z=490.0 [M+H]⁺.

¹H-NMR (400 MHz, DMSO-d6), δ [ppm]=2.04 (1H), 2.30 (1H), 2.91 (1H), 3.03 (1H), 3.12 (1H), 3.21-3.33 (4H*), 4.66 (2H), 8.71 (1H), 8.83 (1H), 8.97 (1H), 10.92 (1H), 12.52 (1H);

*: at least partially hidden by solvent peak.

### Example 101a

### Ethyl (7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylate

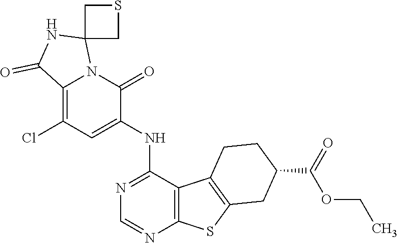 [SMILES: CCOC(=O)[C@H]1CCc2c(sc3ncnc(Nc4cc(Cl)c5n(c4=O)C4(CSC4)NC5=O)c23)C1]

To a solution of 500 mg (1.80 mmol) ethyl (7S)-4-amino-5,6,7,8-tetrahydro[1]benzothieno[2,3-d]pyrimidine-7-carboxylate (prepared according to example 1 b) and 638 mg (1.98 mmol) 6-bromo-8-chloro-2H-spiro[imidazo[1,5-a]pyridine-3,3′-thietane]-1,5-dione (prepared according to example 101b) in 70 mL 1,4-dioxane were added 1.76 g cesium carbonate and the mixture was degassed and purged with argon several times. 112 mg 4,5-bis(diphenylphosphino)-9,9-dimethylxanthene, 92 mg 2-(dicyclohexyl-phosphino)-2′,4′,6′-triisopropylbiphenyl, 43 mg palladium(II)acetate and 177 mg tris(dibenzylideneacetone)dipalladium(0) were added and the mixture was stirred at 100° C. for 2 hours. After concentration the residue was purified by flash chromatography (Biotage SNAP Ultra cartridge silica 100 g, ethanol:dichloromethane) and crystallization from ethanol to give 683 mg (73%) of the title compound.

LC-MS: m/z=518.0 [M+H]⁺.

### Example 101b

### 6-Bromo-8-chloro-2H-spiro[imidazo[1,5-a]pyridine-3,3′-thietane]-1,5-dione

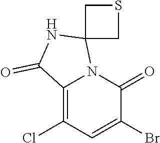 [SMILES: O=C1NC2(CSC2)n2c1c(Cl)cc(Br)c2=O]

To a solution of 3.00 g (11.9 mmol) 5-bromo-3-chloro-6-oxo-1,6-dihydropyridine-2-carboxamide (PCT Int. Appl. (2015), WO 2015200481) and 1.58 g (17.9 mmol) thietan-3-one in 6 mL 1,4-dioxane were added 314 μl sulfuric acid and the mixture was stirred at 95° C. overnight. After concentration water was added, the precipitate crystallited from methanol/dichloromethane and dried to give 1.16 g (30%) of the title compound.

LC-MS: m/z=321.0 [M+H]⁺.

### Example 102

### (7S)-4-[(8-Chloro-1,5-dioxo-1,5-dihydro-2H-spiro[imidazo[1,5-a]pyridine-3,3′-thietan]-6-yl)amino]-N,N-dimethyl-5,6,7,8-tetrahydro[1]benzothieno[2,3-d]pyrimidine-7-carboxamide

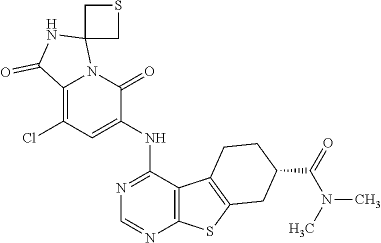 [SMILES: CN(C)C(=O)[C@H]1CCc2c(sc3ncnc(Nc4cc(Cl)c5n(c4=O)C4(CSC4)NC5=O)c23)C1]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 107 μL N-ethyl-N-isopropylpropan-2-amine, 255 μL N-methylmethanamine (2M in tetrahydrofuran) and 182 μL 2,4,6-tripropyl-1,3,5,2,4,6-trioxatriphosphinane 2,4,6-trioxide solution (50% in N,N-dimethylformamide) was stirred at RT overnight. Water was added, the precipitate was washed with water, ethanol and diethylether and dried to give 41 mg (74%) of the title compound.

LC-MS: m/z=517.3 [M+H]⁺.

¹H-NMR (400 MHz, DMSO-d6), δ [ppm]=1.84 (1H), 2.21 (1H), 2.88 (3H), 2.89-3.07 (3H), 3.13 (3H), 3.18-3.31 (4H*), 4.65 (2H), 8.70 (1H), 8.84 (1H), 8.97 (1H), 10.91 (1H);

*: at least partially hidden by solvent peak.

### Example 103

### 6-{[(7S)-7-(Azetidine-1-carbonyl)-5,6,7,8-tetrahydro[1]benzothieno[2,3-d]pyrimidin-4-yl]amino}-8-chloro-2H-spiro[imidazo[1,5-a]pyridine-3,3′-thietane]-1,5-dione

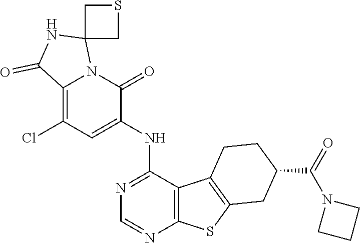 [SMILES: O=C1NC2(CSC2)n2c1c(Cl)cc(Nc1ncnc3sc4c(c13)CC[C@H](C(=O)N1CCC1)C4)c2=O]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 107 μL N-ethyl-N-isopropylpropan-2-amine, 23 mg azetidine and 182 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 48 mg (84%) of the title compound.

LC-MS: m/z=529.1 [M+H]⁺.

¹H-NMR (400 MHz, DMSO-d6), δ [ppm]=1.84 (1H), 2.15-2.28 (3H), 2.78 (1H), 2.94 (2H), 3.18-3.32 (4H), 3.90 (2H), 4.28 (2H), 4.65 (2H), 8.70 (1H), 8.83 (1H), 8.96 (1H), 10.92 (1H);

*: at least partially hidden by solvent peak.

### Example 104

### 1-{(7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carbonyl}azetidine-3-carbonitrile

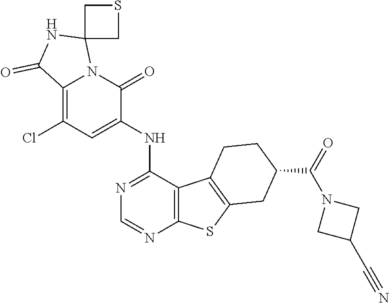 [SMILES: N#CC1CN(C(=O)[C@H]2CCc3c(sc4ncnc(Nc5cc(Cl)c6n(c5=O)C5(CSC5)NC6=O)c34)C2)C1]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 160 μL N-ethyl-N-isopropylpropan-2-amine, 60 mg azetidine-3-carbonitrile hydrochloride and 182 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 41 mg (69%) of the title compound.

LC-MS: m/z=554.3 [M+H]⁺.

¹H-NMR (400 MHz, DMSO-d6), δ [ppm]=1.84 (1H), 2.23 (1H), 2.79 (1H), 2.87-3.06 (2H), 3.15-3.33 (4H*), 3.82 (1H), 4.06 (1H), 4.20 (1H), 4.46-4.70 (4H), 8.70 (1H), 8.83 (1H), 8.96 (1H), 10.92 (1H);

*: at least partially hidden by solvent peak.

### Example 105

### 8-Chloro-6-({(7S)-7-[(3S)-3-(dimethylamino)pyrrolidine-1-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione

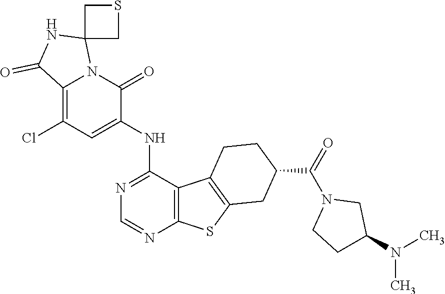 [SMILES: CN(C)[C@H]1CCN(C(=O)[C@H]2CCc3c(sc4ncnc(Nc5cc(Cl)c6n(c5=O)C5(CSC5)NC6=O)c34)C2)C1]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 107 μL N-ethyl-N-isopropylpropan-2-amine, 65 μL (3S)—N,N-dimethylpyrrolidin-3-amine and 182 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 25 mg (40%) of the title compound.

LC-MS: m/z=586.5 [M+H]⁺.

¹H-NMR (400 MHz, DMSO-d6), δ [ppm]=1.56-1.90 (2H), 2.00-2.29 (2H), 2.17 (3H), 2.18 (3H), 2.62+2.75 (1H), 2.91-3.07 (3H), 3.19-3.47 (5H*), 3.47-3.70 (2H), 3.81+3.90 (1H), 4.65 (2H), 8.70 (1H), 8.84 (1H), 8.98 (1H), 10.91 (1H);

*: at least partially hidden by solvent peak.

### Example 106

### 8-Chloro-6-({(7S)-7-[(3R)-3-methylmorpholine-4-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione

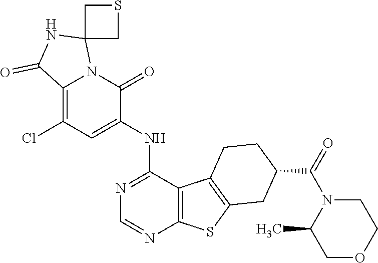 [SMILES: C[C@@H]1COCCN1C(=O)[C@H]1CCc2c(sc3ncnc(Nc4cc(Cl)c5n(c4=O)C4(CSC4)NC5=O)c23)C1]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 30 mL N,N-dimethylacetamide, 107 μL N-ethyl-N-isopropylpropan-2-amine, 58 μL (3R)-3-methylmorpholine and 182 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 42 mg (69%) of the title compound.

LC-MS: m/z=573.4 [M+H]⁺.

¹H-NMR (400 MHz, DMSO-d6), δ [ppm]=1.15+1.34 (3H), 1.91 (1H), 2.17 (1H), 2.82-3.58 (10H*), 3.64 (1H), 3.75-4.48 (3H), 4.65 (2H), 8.70 (1H), 8.84 (1H), 8.97 (1H), 10.92 (1H);

*: at least partially hidden by solvent peak.

### Example 107

### 8-Chloro-6-({(7S)-7-[(3R)-3,4-dimethylpiperazine-1-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione

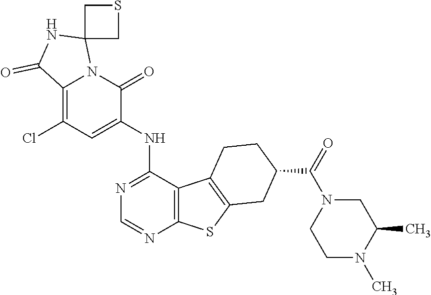 [SMILES: C[C@@H]1CN(C(=O)[C@H]2CCc3c(sc4ncnc(Nc5cc(Cl)c6n(c5=O)C5(CSC5)NC6=O)c34)C2)CCN1C]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 267 μL N-ethyl-N-isopropylpropan-2-amine, 95 mg (2R)-1,2-dimethylpiperazine dihydrochloride and 182 μL 2,4,6-tripropyl-1,3,5,2,4,6-trioxatriphosphinane 2,4,6-trioxide solution (50% in N,N-dimethylformamide) was stirred at 50° C. overnight. 267 μL N-ethyl-N-isopropylpropan-2-amine and 95 mg (2R)-1,2-dimethylpiperazine dihydrochloride were added and stirring continued for 24 hours. Water was added, the precipitate was washed with water, ethanol and diethylether and dried to give 47 mg (71%) of the title compound.

LC-MS: m/z=586.3 [M+H]⁺.

¹H-NMR (400 MHz, DMSO-d6), δ [ppm]=1.01+1.05 (3H), 1.46 (1H), 1.77-2.28 (4H), 2.21 (3H), 2.71-3.30 (9H*), 3.93 (1H), 4.17 (1H), 4.64 (2H), 8.70 (1H), 8.84+8.85 (1H), 8.97 (1H), 10.92 (1H);

*: at least partially hidden by solvent peak.

### Example 108

### 8-Chloro-6-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione

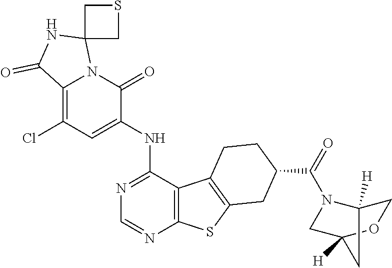 [SMILES: O=C1NC2(CSC2)n2c1c(Cl)cc(Nc1ncnc3sc4c(c13)CC[C@H](C(=O)N1C[C@H]3C[C@@H]1CO3)C4)c2=O]

A mixture of 50 mg (102 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 3.0 mL N,N-dimethylacetamide, 160 μL N-ethyl-N-isopropylpropan-2-amine, 69 mg (1R,4R)-2-oxa-5-azabicyclo[2.2.1]heptane hydrochloride and 182 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 53 mg (87%) of the title compound.

LC-MS: m/z=571.4 [M+H]⁺.

¹H-NMR (400 MHz, DMSO-d6), δ [ppm]=1.75-1.95 (3H), 2.21+2.29 (1H), 2.80-3.14 (3H), 3.19-3.30 (5H*), 3.54-3.80 (3H), 4.56-4.73 (3H), 4.78+4.92 (1H), 8.71 (1H), 8.83+8.84 (1H), 8.98+9.00 (1H), 10.92 (1H);

*: at least partially hidden by solvent peak.

### Example 109

### (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid

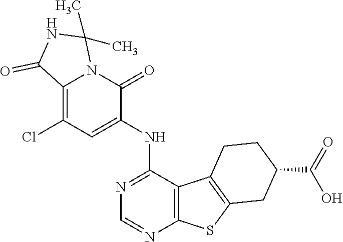 [SMILES: CC1(C)NC(=O)c2c(Cl)cc(Nc3ncnc4sc5c(c34)CC[C@H](C(=O)O)C5)c(=O)n21]

A mixture of 605 mg (1.24 mmol) ethyl (7S)-4-[(8-chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylate (prepared according to example 109a) in 19 mL tetrahydrofuran, 9 mL ethanol and 7.4 mL lithium hydroxide solution (1M in water) was stirred at RT for 4 hours. Water was added and the mixture was acidified with hydrochloric acid. After concentration the precipitate was washed with water and dried to give 586 mg (98%) of the title compound.

LC-MS: m/z=460.1 [M+H]⁺.

¹H-NMR (400 MHz, DMSO-d6), δ [ppm]=1.82 (6H), 1.99 (1H), 2.25 (1H), 2.87 (1H), 3.00 (1H), 3.10 (1H), 3.14-3.26 (2H), 8.67 (1H), 8.83 (1H), 8.92 (1H), 9.84 (1H), 12.51 (1H).

### Example 109a

### Ethyl (7S)-4-[(8-chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylate

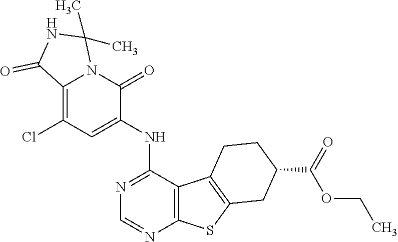 [SMILES: CCOC(=O)[C@H]1CCc2c(sc3ncnc(Nc4cc(Cl)c5n(c4=O)C(C)(C)NC5=O)c23)C1]

To a solution of 500 mg (1.80 mmol) ethyl (7S)-4-amino-5,6,7,8-tetrahydro[1]benzothieno[2,3-d]pyrimidine-7-carboxylate (prepared according to example 1 b) and 578 mg (1.98 mmol) 6-bromo-8-chloro-3,3-dimethyl-2,3-dihydroimidazo[1,5-a]pyridine-1,5-dione (prepared according to example 109b) in 70 mL 1,4-dioxane were added 1.76 g cesium carbonate and the mixture was degassed and purged with argon several times. 112 mg 4,5-bis(diphenylphosphino)-9,9-dimethylxanthene, 92 mg 2-(dicyclohexyl-phosphino)-2′,4′,6′-triisopropylbiphenyl, 43 mg palladium(II)acetate and 177 mg tris(dibenzylideneacetone)dipalladium(0) were added and the mixture was stirred at 100° C. for 2 hours. After concentration the residue was purified by flash chromatography (Biotage SNAP Ultra cartridge silica 100 g, ethanol:dichloromethane) and crystallization from ethanol to give 607 mg (69%) of the title compound.

LC-MS: m/z=488.4 [M+H]⁺.

### Example 109b

### 6-Bromo-8-chloro-3,3-dimethyl-2,3-dihydroimidazo[1,5-a]pyridine-1,5-dione

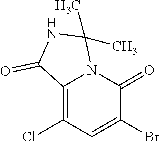

To a solution of 3.00 g (11.9 mmol) 5-bromo-3-chloro-6-oxo-1,6-dihydropyridine-2-carboxamide (PCT Int. Appl. (2015), WO 2015200481) and 3.51 mL (47.7 mmol) acetone in 35 mL 1,4-dioxane were added 314 μl sulfuric acid and the mixture was stirred at 95° C. for 3 hours. After concentration water was added, the precipitate washed with water and diethyl ether and dried to give 2.98 g (86%) of the title compound.

LC-MS: m/z=290.9 [M+H]⁺.

### Example 110

### (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxamide

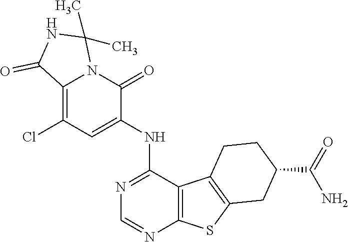 [SMILES: CC1(C)NC(=O)c2c(Cl)cc(Nc3ncnc4sc5c(c34)CC[C@H](C(N)=O)C5)c(=O)n21]

A mixture of 80 mg (174 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 5.5 mL N,N-dimethylacetamide, 182 μL N-ethyl-N-isopropylpropan-2-amine, 4.4 mL ammonia (0.4M in tetrahydrofurane) and 311 μL 2,4,6-tripropyl-1,3,5,2,4,6-trioxatriphosphinane 2,4,6-trioxide solution (50% in N,N-dimethylformamide) was stirred at 60° C. overnight. 4.4 mL ammonia (0.4M in tetrahydrofurane) and 311 μL 2,4,6-tripropyl-1,3,5,2,4,6-trioxatriphosphinane 2,4,6-trioxide solution (50% in N,N-dimethylformamide) were added and stirring continued for 2.5 days. Water was added, the precipitate was washed with water and ethanol and dried to give 66 mg (79%) of the title compound.

LC-MS: m/z=459.3 [M+H]⁺.

¹H-NMR (400 MHz, DMSO-d6), δ [ppm]=1.82 (6H), 1.90 (1H), 2.21 (1H), 2.68 (1H), 2.91-3.04 (2H), 3.14 (1H), 3.26 (1H), 6.99 (1H), 7.48 (1H), 8.69 (1H), 8.85 (1H), 8.97 (1H), 9.83 (1H).

### Example 111

### (7S)-4-[(8-Chloro-3,3-dimethyl-1,5-dioxo-1,2,3,5-tetrahydroimidazo[1,5-a]pyridin-6-yl)amino]-N,N-dimethyl-5,6,7,8-tetrahydro[1]benzothieno[2,3-d]pyrimidine-7-carboxamide

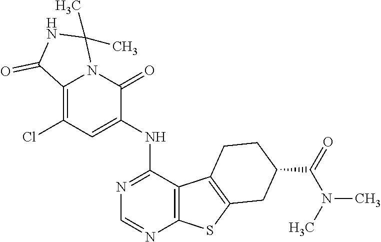 [SMILES: CN(C)C(=O)[C@H]1CCc2c(sc3ncnc(Nc4cc(Cl)c5n(c4=O)C(C)(C)NC5=O)c23)C1]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 114 μL N-ethyl-N-isopropylpropan-2-amine, 272 μL N-methylmethanamine (2M in tetrahydrofuran) and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 47 mg (83%) of the title compound.

LC-MS: m/z=487.4 [M+H]⁺.

¹H-NMR (400 MHz, DMSO-d6), δ [ppm]=1.82 (7H), 2.16 (1H), 2.88 (3H), 2.91-3.05 (2H), 3.11 (3H), 3.16-3.28 (3H), 8.70 (1H), 8.88 (1H), 8.98 (1H), 9.84 (1H).

### Example 112

### 6-{[(7S)-7-(Azetidine-1-carbonyl)-5,6,7,8-tetrahydro[1]benzothieno[2,3-d]pyrimidin-4-yl]amino}-8-chloro-3,3-dimethyl-2,3-dihydroimidazo[1,5-a]pyridine-1,5-dione

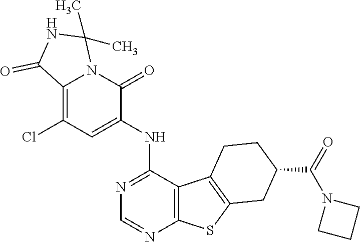 [SMILES: CC1(C)NC(=O)c2c(Cl)cc(Nc3ncnc4sc5c(c34)CC[C@H](C(=O)N3CCC3)C5)c(=O)n21]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 114 μL N-ethyl-N-isopropylpropan-2-amine, 25 mg azetidine hydrochloride and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 48 mg (84%) of the title compound.

LC-MS: m/z=499.1 [M+H]⁺.

¹H-NMR (400 MHz, DMSO-d6), δ [ppm]=1.75-1.89 (7H), 2.14 (1H), 2.22 (2H), 2.77 (1H), 2.93 (2H), 3.13-3.28 (2H), 3.89 (2H), 4.26 (2H), 8.70 (1H), 8.87 (1H), 8.97 (1H), 9.84 (1H).

### Example 113

### 1-{(7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carbonyl}azetidine-3-carbonitrile

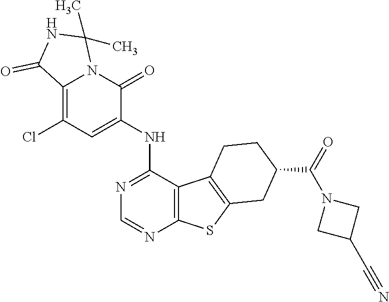 [SMILES: CC1(C)NC(=O)c2c(Cl)cc(Nc3ncnc4sc5c(c34)CC[C@H](C(=O)N3CC(C#N)C3)C5)c(=O)n21]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 170 μL N-ethyl-N-isopropylpropan-2-amine, 64 mg azetidine-3-carbonitrile and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 46 mg (77%) of the title compound.

LC-MS: m/z=524.4 [M+H]⁺.

¹H-NMR (400 MHz, DMSO-d6), δ [ppm]=1.82 (7H), 2.17 (1H), 2.78 (1H), 2.85-3.07 (2H), 3.14-3.26 (2H), 3.82 (1H), 4.05 (1H), 4.19 (1H), 4.52 (2H), 8.70 (1H), 8.87 (1H), 8.97 (1H), 9.83 (1H).

### Example 114

### 8-Chloro-6-({(7S)-7-[(3S)-3-(dimethylamino)pyrrolidine-1-carbonyl]-5,6,7,8-tetrahydro[1]benzothieno[2,3-d]pyrimidin-4-yl}amino)-3,3-dimethyl-2,3-dihydroimidazo[1,5-a]pyridine-1,5-dione

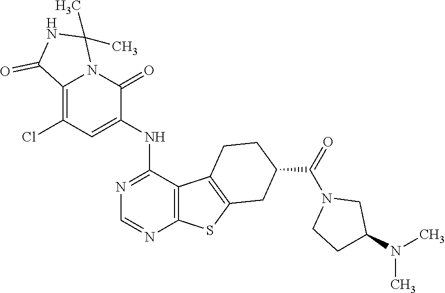 [SMILES: CN(C)[C@H]1CCN(C(=O)[C@H]2CCc3c(sc4ncnc(Nc5cc(Cl)c6n(c5=O)C(C)(C)NC6=O)c34)C2)C1]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 114 μL N-ethyl-N-isopropylpropan-2-amine, 69 μL (3S)—N,N-dimethylpyrrolidin-3-amine and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 50 mg (78%) of the title compound.

LC-MS: m/z=556.2 [M+H]⁺.

¹H-NMR (400 MHz, DMSO-d6), δ [ppm]=1.57-1.88 (2H), 1.81+1.82 (6H), 1.97-2.23 (2H), 2.16 (3H), 2.17 (3H), 2.61+2.73 (1H), 2.90-3.07 (3H), 3.15-3.66 (5H*), 3.78+3.86 (1H), 8.69 (1H), 8.86+8.87 (1H), 8.97 (1H), 9.82 (1H);

*: at least partially hidden by solvent peak.

### Example 115

### 8-Chloro-6-({(7S)-7-[(3R)-3,4-dimethylpiperazine-1-carbonyl]-5,6,7,8-tetrahydro[1]benzothieno[2,3-d]pyrimidin-4-yl}amino)-3,3-dimethyl-2,3-dihydroimidazo[1,5-a]pyridine-1,5-dione

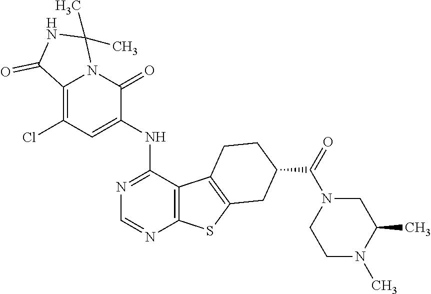 [SMILES: C[C@@H]1CN(C(=O)[C@H]2CCc3c(sc4ncnc(Nc5cc(Cl)c6n(c5=O)C(C)(C)NC6=O)c34)C2)CCN1C]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 284 μL N-ethyl-N-isopropylpropan-2-amine, 102 mg (2R)-1,2-dimethylpiperazine dihydrochloride and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 58 mg (91%) of the title compound.

LC-MS: m/z=556.5 [M+H]⁺.

¹H-NMR (400 MHz, DMSO-d6), δ [ppm]=1.00+1.03 (3H), 1.73-2.23 (4H), 1.82 (6H), 2.19 (3H), 2.74 (1H), 2.80-3.09 (3H), 3.16-3.29 (4H), 3.91 (1H), 4.17 (1H), 8.69 (1H), 8.85+8.86 (1H), 8.95 (1H), 9.84 (1H).

### Example 116

### 8-Chloro-3,3-dimethyl-6-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2,3-dihydroimidazo[1,5-a]pyridine-1,5-dione

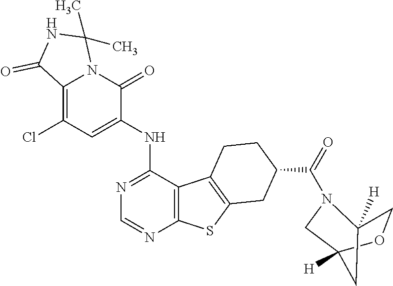 [SMILES: CC1(C)NC(=O)c2c(Cl)cc(Nc3ncnc4sc5c(c34)CC[C@H](C(=O)N3C[C@H]4C[C@@H]3CO4)C5)c(=O)n21]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 170 μL N-ethyl-N-isopropylpropan-2-amine, 74 mg (1R,4R)-2-oxa-5-azabicyclo[2.2.1]heptane hydrochloride and 194 μL 2,4,6-tripropyl-1,3,5,2,4,6-trioxatriphosphinane 2,4,6-trioxide solution (50% in N,N-dimethylformamide) was stirred at 50° C. overnight. Water was added, the precipitate was washed with water, ethanol and diethylether and dried to give 53 mg (86%) of the title compound.

LC-MS: m/z=541.4 [M+H]⁺.

¹H-NMR (400 MHz, DMSO-d6), δ [ppm]=1.76-1.94 (2H), 1.82 (6H), 2.15+2.23 (1H), 2.79-3.30 (7H), 3.56 (1H), 3.62-3.78 (2H), 4.61+4.66 (1H), 4.78+4.90 (1H), 8.70 (1H), 8.86+8.87 (1H), 8.98+8.99 (1H), 9.84 (1H).

### Example 117

### 8-chloro-3,3-dimethyl-6-({(7S)-7-[(3R)-3-methylmorpholine-4-carbonyl]-5,6,7,8-tetrahydro[1]benzothieno[2,3-d]pyrimidin-4-yl}amino)-2,3-dihydroimidazo[1,5-a]pyridine-1,5-dione

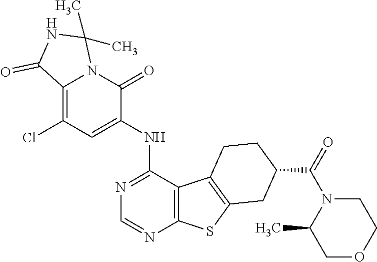 [SMILES: C[C@@H]1COCCN1C(=O)[C@H]1CCc2c(sc3ncnc(Nc4cc(Cl)c5n(c4=O)C(C)(C)NC5=O)c23)C1]

A mixture of 50 mg (109 μmol) (7S)-4-[(8-Chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid (prepared according to example 109), 3.0 mL N,N-dimethylacetamide, 114 μL N-ethyl-N-isopropylpropan-2-amine, 55 mg (3R)-3-methylmorpholine and 194 μL 2,4,6-tripropyl-1,3,5,2,4,6-trioxatriphosphinane 2,4,6-trioxide solution (50% in ethyl acetate) was stirred at 50° C. overnight. Water and ethanol were added, the formed precipitate was filtered off, washed with water and ethanol and dried. The obtained material was triturated with dichloromethane and ethanol, isolated by centrifugation and dried to give 37 mg (59%) of the title compound.

LC-MS: m/z=543.1 [M+H]⁺.

¹H-NMR (400 MHz, DMSO-d6), δ [ppm]=1.14-1.23 (2H), 1.32-1.35 (1H), 1.81-1.95 (7H), 2.08-2.17 (1H), 2.88-3.26 (6H), 3.37-3.55 (2H*), 3.61-3.66 (1H), 3.76-4.45 (3H), 8.69 (1H), 8.85+8.87 (1H), 8.96 (1H), 9.84 (1H).

*: at least partially hidden by water peak.

### Example 118

### 8′-chloro-6′-({(7S)-7-[(1R,4R)-2-oxa-5-azabicyclo[2.2.1]heptan-5-ylcarbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione

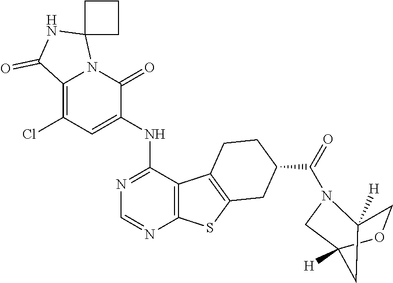 [SMILES: O=C1NC2(CCC2)n2c1c(Cl)cc(Nc1ncnc3sc4c(c13)CC[C@H](C(=O)N1C[C@H]3C[C@@H]1CO3)C4)c2=O]

A mixture of 50 mg (106 μmol) (7S)-4-[(8′-Chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid (prepared according to example 93), 3.0 mL N,N-dimethylacetamide, 166 μL N-ethyl-N-isopropylpropan-2-amine, 72 mg (1R,4R)-2-oxa-5-azabicyclo[2.2.1]heptane hydrochloride and 189 μL 2,4,6-tripropyl-1,3,5,2,4,6-trioxatriphosphinane 2,4,6-trioxide solution (50% in ethyl acetate) was stirred at 50° C. overnight. Water and ethanol were added, the formed precipitate was filtered off, washed with water, ethanol and diethylether and dried. The obtained material was triturated with dichloromethane and ethanol, isolated by centrifugation and dried to give 47 mg (73%) of the title compound.

LC-MS: m/z=553.4 [M+H]⁺.

¹H-NMR (400 MHz, DMSO-d6), δ [ppm]=1.23 (4H), 1.79-2.01 (3H), 2.10-2.37 (2H), 2.78-3.30 (5H*), 3.41-3-50 (2H), 3.55-3.60 (1H), 3.65-3.77 (2H), 4.62+4.67 (1H), 4.78+4.92 (1H), 8.69 (1H), 8.82-8.84 (1H), 8.96-8.97 (1H), 10.37 (1H).

*: at least partially hidden by water peak.

### Example 119

### (7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxamide

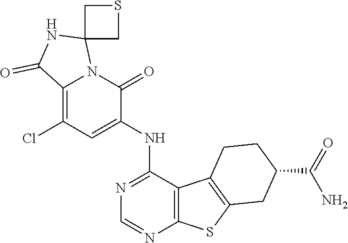 [SMILES: NC(=O)[C@H]1CCc2c(sc3ncnc(Nc4cc(Cl)c5n(c4=O)C4(CSC4)NC5=O)c23)C1]

A mixture of 80 mg (163 μmol) (7S)-4-[(8-Chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid (prepared according to example 101), 5.0 mL N,N-dimethylacetamide, 171 μL N-ethyl-N-isopropylpropan-2-amine, 4.1 mL ammonia (0.4M in tetrahydrofurane) and 292 μL 2,4,6-tripropyl-1,3,5,2,4,6-trioxatriphosphinane 2,4,6-trioxide solution (50% in ethyl acetate) was stirred at 60° C. overnight. Another 4.1 mL ammonia (0.4M in tetrahydrofurane) and 292 μL 2,4,6-tripropyl-1,3,5,2,4,6-trioxatriphosphinane 2,4,6-trioxide solution (50% in ethyl acetate) were added and stirring at 60° C. continued for 3 days. The mixture was concentrated in vacuo and treated with another 4.1 mL ammonia (0.4M in tetrahydrofurane) and 292 μL 2,4,6-tripropyl-1,3,5,2,4,6-trioxatriphosphinane 2,4,6-trioxide solution (50% in ethyl acetate) and stirred at 60° C. overnight. Water and ethanol were added, the formed precipitate was filtered off, washed with water and ethanol and dried. The obtained material was triturated with dichloromethane and ethanol, isolated by centrifugation and dried to give 57 mg (67%) of the title compound.

LC-MS: m/z=489.0 [M+H]⁺.

¹H-NMR (400 MHz, DMSO-d6), δ [ppm]=1.8-1-99 (1H), 2.24-2.28 (1H), 2.69-2.74 (1H), 2.93-3.05 (2H), 3.28-3-29 (4H*), 4.641-4.68 (2H), 7.00 (1H), 7.48 (1H), 8.70 (1H), 8.79-8.82 (1H), 8.98 (1H), 10.91 (1H).

*: at least partially hidden by water peak.

Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃) air displacement agents (examples include but are not limited to nitrogen and argon); antifungal preservatives (examples include but are not limited to benzoic acid, butylparaben, ethylparaben, methylparaben, propylparaben, sodium benzoate); antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax); suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows: Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilised powder for IV administration: A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, topoisomerase inhibitors, proteasome inhibitors, biological response modifiers, anti-hormones or agents used for the treatment of inflammatory diseases or pain disorders.

The terms “chemotherapeutic agent” and anti-cancer agent”, include but are not limited to 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, BAY 86-9766 (RDEA 119), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, deforolimus, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, enzastaurin, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, larotaxel, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, novolimus, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, perifosine, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, rapamycin, razoxane, regorafenib, risedronic acid, rituximab, romidepsin, romiplostim, sagopilone, sargramostim, selumetinib, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trametinib, trastuzumab, treosulfan, tretinoin, triciribine, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin, zotarolimus, ARRY-162, ARRY-300, ARRY-704, AS-703026, AZD-5363, AZD-8055, BEZ-235, BGT-226, BKM-120, BYL-719, CAL-101, CC-223, CH-5132799, E-6201, GDC-0032, GDC-0068, GDC-0623, GDC-0941, GDC-0973, GDC-0980, GSK-2110183, GSK-2126458, GSK-2141795, INK128, MK-2206, OSI-027, PF-04691502, PF-05212384, PX-866, RG-7167, RO-4987655, RO-5126766, TAK-733, UCN-01, WX-554, XL-147, XL-765, ZSTK-474.

The terms “chemotherapeutic agent” and anti-cancer agent”, also include protein therapeutics such as an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-llinked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I -chTNT-1/B.

The terms “chemotherapeutic agent” and “anti-cancer agent”, also include monoclonal antibodies useful as the protein therapeutic such as muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemo-therapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

Beyond the “chemotherapeutic agent” and “anti cancer agent” the invention can be combined with further “anti-inflammatory” and “anti-pain agents” which include but are not limited to abatacept, or anti-bacterial agents (e.g. penicilline, vancomycin, ciprofloxacin), anti-viral agents (e.g. aciclovir, oseltamivir), anti-mycotic agents (e.g. naftifine, nystatin), azathioprine, belimumab, corticosteroids (e.g. prednisone, prednisolone, methylprednisolone, hydrocortisone, betamethasone), cyclophosphamide, IgE antibody, immunoglobulin and gammaglobuline, IL-1 inhibitors (e.g. anakinra, canakinumab, rilonacept), “immunomodulatory and immunosuppressive agents” like cyclosporine, mercaptopurine, Methotrexat®; interferon including beta-interferon (IFN beta-1a: Avonex® and IFN beta-1b: Betaferon®), Jak/STAT inhibitors (e.g. tofacitinib, baricitinib, GLPG0634), leflunomide, mycophenolic acid, nonsteroidal anti-inflammatory drugs (NSAIDS) (e.g. ibuprofen, naproxen, etodolac, celecoxib, colchicine), paracetamol, phosphodiesterase-inhibitor (e.g. apremilast, roflumilast), rapamycin, rituximab, sulfasalazine, tacrolimus and TNF-antagonist (e.g. Humira®, etanercept, infliximab).

In addition, combination also includes ACE (angiotensin-converting-enzyme) inhibitors (e.g. benazepril), acetylsalicylic acid, acetylcholinesterase inhibitors (e.g. donepezil, rivastigmine, galantamine, tacrine), anticholinergic agents (e.g. trihexyphenidyle, glycopyrronium bromid), anticonvulsant agents (e.g. gabapentin), anti-diarrhoeal drug (e.g. loperamide or laxatives), antileukotriene agents (e.g. montelukast), beta blocker (e.g. metoprolol), beta2-adrenergic agonists (e.g. salbutamol), calcium channel blockers (e.g. nifedipine), chloroquine, COMT (Catechol-O-Methyltransferase)-inhibitors (e.g. entacapone), diuretics (e.g. hydrochlorothiazide), dopamine agonists (e.g. ropinrole, pramipexole, bromocriptine), efalizumab, fingolimod, glatiramer acetate, glibenclamide, insulin therapy, L-DOPA/Carbidopa (L-3,4-Dihydroxyphenylalanin), MAO-B (monoamine oxidase B) inhibitors (e.g. selegiline), mesalazine, metformin, methylxanthine drugs (e.g. theophylline), mitoxantrone, natalizumab, NMDA (N-Methyl-D-Aspartat) receptor antagonists (e.g. amantadine, memantine), probiotics (e.g. mutaflor, VSL#3®, Lactobacillus GG, Lactobacillus plantarum, L. acidophilus, L. casei, Bifidobacterium infantis 35624, Enterococcus fecium SF68, Bifidobacterium longum), statin (e.g. simvastatin), sulfonylureas (e.g. tolnutamide, glimepiride), urea and vitamin-D analoga (e.g. calcipotriol, calcitriol, tacalcitol).

Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit MKNK1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof, or pancreatitis.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant serin threonin kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

Methods of Treating Pain-Associated Diseases and Gynaecological Disorders.

The present invention also provides methods for the treatment or prophylaxis of inflammation and pain-associated diseases.

In particular aspect of the invention as reported above a compound of formula (I), (Ia) or (Ib) is for the treatment of pain syndromes including acute, chronic, inflammatory and neuropathic pain, preferably inflammatory pain, surgical pain, visceral pain, dental pain, premenstrual pain, gynaecological disease, preferably dysmenorrhea, dyspareunia or endometriosis, adenomyosis, endometriosis-associated pain, or other endometriosis-associated symptoms, wherein said symptoms are in particular endometriosis-associated dysmenorrhea, dyspareunia, dysuria, or dyschezia, pain associated with fibrotic diseases, central pain, pain due to burning mouth syndrome, pain due to burns, pain due to migraine, cluster headaches, pain due to nerve injury, pain due to neuritis, neuralgias, pain due to poisoning, pain due to ischemic injury, pain due to interstitial cystitis, cancer pain, pain due to viral, parasitic or bacterial infections, pain due to traumatic nerve-injury, pain due to post-traumatic injuries (including fractures and sport injuries), pain due to trigeminal neuralgia, pain associated with small fiber neuropathy, pain associated with diabetic neuropathy, chronic lower back pain, phantom limb pain, pelvic pain syndrome, chronic pelvic pain, neuroma pain, complex regional pain syndrome, pain associated with gastrointestinal distension, chronic arthritic pain and related neuralgias, and pain associated with cancer, pain associated with chemotherapy, HIV and HIV treatment-induced neuropathy; and pain associated with diseases or disorders selected from the group consisting of hyperalgesia, allodynia, irritable bowel syndrome.

In addition, the present invention is for the use of the treatment and prevention of inflammatory diseases including inflammatory bowel disease (ulcerative colitis and Crohn's disease), hyperaemia, sepsis, metabolic disorders, e.g. obesity, insulin resistance, diabetes mellitus type 1 and 2, metabolic endocrine disorder; metabolic syndrome; atherosclerosis, reperfusion injury, inflammatory bone resorption, inflammatory liver diseases, pulmonary fibrosis, acute respiratory distress syndrome, and intestinal polyposis, inflammatory skin diseases like psoriasis, pemphigus vulgaris, inflammatory eye disorders like non-infectious uveitis, primary and secondary autoimmune uveitis, VKH syndrome, anterior uveitis, intermediate uveitis, posterior uveitis, panuveitis, Behcet's disease and neuromyelitis optica, fibrotic diseases like idiopathic pulmonary fibrosis, skin fibrosis, systemic sclerosis, autism disorders, liver diseases like nonalcoholic-, alcoholic- and toxic fatty liver disease, steatohepatitis, hepatic fibrosis; and cirrhosis; lung diseases like chronic obstructive pulmonary disease, asthma, pneumonia; neurodegenerative diseases like Parkinson's disease, Alzheimer's disease; stroke, postischemic brain injury, brain ischemia, post-traumatic brain injury, alopecia, acute coronary syndrome, myocardial infarction, autoimmune diseases like autoimmune encephalomyelitis, multiple sclerosis; arthritis (such as osteoarthritis and rheumatoid arthritis); psoriatic arthritis, ankylosing spondylitis), psoriasis, lupus erythematosus (e.g. systemic lupus erythematosus, cutaneous lupus and neonatal lupus erythematosus); inflammatory, autoimmune and fibrotic kidney diseases (e.g. glomerulonephritis, lupus nephritis, ANCA) interstitial cystitis, hypertrophy of the e.g. kidney, ischemia/reperfusion injury, allergic rhinitis, burn wound, osteoporosis viral and bacterial infections, chemotherapy-induced alopecia, cachexia induced for any reason, e.g. cancer, heart failure, etc., muscular atrophy, pancreatitis, schizophrenia, seizures, epilepsy, Fragile X syndrome, graft-versus-host disease, graft rejection, heart fibrosis, autoimmune myocardial disease, myocarditis, endocarditis, ischemia-reperfusion injury following e.g. myocardial infarction, hypertension, artherosclerosis, acute lung injury, ARDS, hypersensitivity pneumonitis, lung fibrosis, e.g. idiopathic pulmonary fibrosis, lymphocytic bronchiolitis e.g. after lung transplantation, dry eye, subfertility (e.g. associated with inflammatory conditions such as endometriosiso, or metabolic endocrine disorders).

Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

Biological Assays Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

MKNK1 Kinase Assay

MKNK1-inhibitory activity of compounds of the present invention was quantified employing the MKNK1 TR-FRET assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (0.1 μM=>final conc. in the 5 μL assay volume is 0.06 μM) in assay buffer and the resulting mixture was incubated for a reaction time of 45 min at 22° C. The concentration of MKNK1 was adjusted depending of the activity of the enzyme lot and was chosen appropriate to have the assay in the linear range, typical concentrations were in the range of 0.05 μg/ml. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values were calculated by a 4 parameter fit using an inhouse software.

MKNK1 Kinase High ATP Assay

MKNK1-inhibitory activity at high ATP of compounds of the present invention after their preincubation with MKNK1 was quantified employing the TR-FRET-based MKNK1 high ATP assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used, which can be purchased e.g. from the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 3.3 mM=>final conc. in the 5 μL assay volume is 2 mM) and substrate (0.1 μM=>final conc. in the 5 μL assay volume is 0.06 μM) in assay buffer and the resulting mixture was incubated for a reaction time of 30 min at 22° C. The concentration of MKNK1 was adjusted depending of the activity of the enzyme lot and was chosen appropriate to have the assay in the linear range, typical concentrations were in the range of 0.003 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated. Data are presented in Table 1.

TABLE 1

| | MKNK1 |
|---|---|
| Example | IC₅₀ [nM] |
| 1 | 9.0 |
| 2 | 0.2 |
| 3 | 0.3 |
| 4 | 0.4 |
| 5 | 1.1 |
| 6 | 1.5 |
| 7 | 4.2 |
| 8 | 0.6 |
| 9 | 0.1 |
| 10 | 0.3 |
| 11 | 0.5 |
| 12 | 0.2 |
| 13 | 0.4 |
| 14 | 0.8 |
| 15 | 15.6 |
| 16 | 3.9 |
| 17 | 0.5 |
| 18 | 0.3 |
| 19 | 0.9 |
| 20 | 0.6 |
| 21 | 0.6 |
| 22 | 0.5 |
| 23 | 0.4 |
| 24 | 0.2 |
| 25 | 0.4 |
| 26 | 0.7 |
| 27 | 2.0 |
| 28 | 1.0 |
| 29 | 1.0 |
| 30 | 1.3 |
| 31 | 0.9 |
| 32 | 0.5 |
| 33 | 0.8 |
| 34 | 4.7 |
| 35 | 0.4 |
| 36 | 1.3 |
| 37 | 0.7 |
| 38 | 3.1 |
| 39 | 3.3 |
| 40 | 0.3 |
| 41 | 1.2 |
| 42 | 24.5 |
| 43 | 9.9 |
| 44 | 17.4 |
| 45 | 0.6 |
| 46 | 0.6 |
| 47 | 0.8 |
| 48 | 0.4 |
| 49 | 0.5 |
| 50 | 0.3 |
| 51 | 0.4 |
| 52 | 0.6 |
| 53 | 13.5 |
| 54 | 4.4 |
| 55 | 0.6 |
| 56 | 2.0 |
| 57 | 1.4 |
| 58 | 0.5 |
| 59 | 37.9 |
| 60 | 9.5 |
| 61 | 8.4 |
| 62 | 6.5 |
| 63 | 1.6 |
| 64 | 3.8 |
| 65 | nd |
| 66 | 3.1 |
| 67 | 2.0 |
| 68 | 2.5 |
| 69 | 2.0 |
| 70 | 0.8 |
| 71 | 0.8 |
| 72 | 0.7 |
| 73 | 1.6 |
| 74 | 0.9 |
| 75 | 1.7 |
| 76 | 0.9 |
| 77 | 0.8 |
| 78 | 4.8 |
| 79 | 0.3 |
| 80 | 0.2 |
| 81 | 0.3 |
| 82 | 1.5 |
| 83 | 1.5 |
| 84 | 1.0 |
| 85 | 0.2 |
| 86 | 0.1 |
| 87 | 0.2 |
| 88 | <0.1 |
| 89 | <0.1 |
| 90 | 0.3 |
| 91 | <0.1 |
| 92 | 1.0 |
| 93 | 1.3 |
| 94 | 0.2 |
| 95 | 1.9 |
| 96 | 0.1 |
| 97 | 0.1 |
| 98 | <0.1 |
| 99 | 0.1 |
| 100 | 0.1 |
| 101 | 1.8 |
| 102 | 0.2 |
| 103 | 0.2 |
| 104 | 0.2 |
| 105 | 0.1 |
| 106 | 0.3 |
| 107 | 0.2 |
| 108 | 0.8 |
| 109 | 26.7 |
| 110 | 1.2 |
| 111 | 0.3 |
| 112 | 0.2 |
| 113 | 0.2 |
| 114 | <0.1 |
| 115 | 0.2 |
| 116 | 0.3 |
| 117 | 0.4 |
| 118 | 0.4 |
| 119 | 0.9 |

MKNK 2 Kinase High ATP Assay

MKNK 2-inhibitory activity at high ATP of compounds of the present invention after their preincubation with MKNK 2 was quantified employing the TR-FRET-based MKNK 2 high ATP assay as described in the following paragraphs.

A recombinant fusion protein of Glutathione-S-Transferase (GST, N-terminally) and human full-length MKNK 2 (Genbank accession number NP_060042.2), expressed in insect cells using baculovirus expression system, purified via glutathione sepharose affinity chromatography, and activated in vitro with MAPK12, was purchased from Invitrogen (product no PV5608) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nl of a 100 fold concentrated solution of the test compound in DMSO was pipetted into a black low volume 384 well microtiter plate (Greiner Bio-One, Frickenhausen, Germany), 2 μl of a solution of MKNK 2 in aqueous assay buffer [50 mM HEPES pH 7.5, 5 mM MgCl₂, 1.0 mM dithiothreitol, 0.005% (v/v) Nonidet-P40 (G-Biosciences, St. Louis, USA)] was added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 3.3 mM=>final conc. in the 5 μl assay volume is 2 mM) and substrate (0.1 μM=>final conc. in the 5 μl assay volume is 0.06 μM) in assay buffer and the resulting mixture was incubated for a reaction time of 30 min at 22° C. The concentration of MKNK 2 was adjusted depending of the activity of the enzyme lot and was chosen appropriate to have the assay in the linear range, typical concentrations were in the range of 0.0045 μg/ml. The reaction was stopped by the addition of 5 μl of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL665. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition).

Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated.

EGFR Kinase Assay

EGFR inhibitory activity of compounds of the present invention was quantified employing the TR-FRET based EGFR assay as described in the following paragraphs.

Epidermal Growth Factor Receptor (EGFR) affinity purified from human carcinoma A431 cells (Sigma-Aldrich, # E3641) was used as kinase. As substrate for the kinase reaction the biotinylated peptide biotin-Ahx-AEEEEYFELVAKKK (C-terminus in amid form) was used which can be purchased e.g. form the company Biosynthan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of EGFR in aqueous assay [50 mM Hepes/HCl pH 7.0, 1 mM MgCl₂, 5 mM MnCl₂, 0.5 mM activated sodium ortho-vanadate, 0.005% (v/v) Tween-20] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration were in the range of 3 U/ml. The reaction was stopped by the addition of 5 μl of a solution of HTRF detection reagents (0.1 μM streptavidine-XL665 [Cis Biointernational] and 1 nM PT66-Tb-Chelate, an terbium-chelate labelled anti-phospho-tyrosine antibody from Cis Biointernational [instead of the PT66-Tb-chelate PT66-Eu-Cryptate from Perkin Elmer can also be used]) in an aqueous EDTA-solution (80 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Eu-Chelate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated.

CDK2/CycE Kinase Assay

CDK2/CycE inhibitory activity of compounds of the present invention can be quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, can be purchased from ProQinase GmbH (Freiburg, Germany). As substrate for the kinase reaction biotinylated peptide biotin-Ttds-YISPLKSPYKISEG (C-terminus in amid form) can be used which can be purchased e.g. from the company JERINI peptide technologies (Berlin, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of CDK2/CycE in aqueous assay buffer [50 mM Tris/HCl pH 8.0, 10 mM MgCl₂, 1.0 mM dithiothreitol, 0.1 mM sodium ortho-vanadate, 0.01% (v/v) Nonidet-P40 (Sigma)] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.25 μM=>final conc. in the 5 μL assay volume is 0.75 μM) in assay buffer and the resulting mixture is incubated for a reaction time of 25 min at 22° C. The concentration of CDK2/CycE is adjusted depending of the activity of the enzyme lot and is chosen appropriate to have the assay in the linear range, typical concentrations ae in the range of 130 ng/ml. The reaction is stopped by the addition of 5 μL of a solution of TR-FRET detection reagents (0.2 μM streptavidine-XL665 [Cisbio Bioassays, Codolet, France] and 1 nM anti-RB(pSer807/pSer811)-antibody from BD Pharmingen [#558389] and 1.2 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.2% (w/v) bovine serum albumin in 100 mM HEPES/NaOH pH 7.0).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL. Therefore, the fluorescence emissions at 620 nm and 665 nm after excitation at 350 nm is measured in a TR-FRET reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

PDGFRβ Kinase Assay

PDGFRβ inhibitory activity of compounds of the present invention can be quantified employing the PDGFRβ HTRF assay as described in the following paragraphs.

As kinase, a GST-His fusion protein containing a C-terminal fragment of human PDGFRβ (amino acids 561-1106, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GT0BLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of PDGFRβ in aqueous assay buffer [50 mM HEPES/NaOH pH 7.5, 10 mM MgCl₂, 2.5 mM dithiothreitol, 0.01% (v/v) Triton-X100 (Sigma)] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [˜30 nM]) in assay buffer and the resulting mixture is incubated for a reaction time of 25 min at 22° C. The concentration of PDGFRβ in the assay is adjusted depending of the activity of the enzyme lot and is chosen appropriate to have the assay in the linear range, typical enzyme concentrations are in the range of about 125 pg/μL (final conc. in the 5 μL assay volume). The reaction is stopped by the addition of 5 μL of a solution of HTRF detection reagents (200 nM streptavidine-XLent [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XLent and the PT66-Eu-Chelate. Subsequently the amount of phosphorylated substrate is evaluated by measurement of the resonance energy transfer from the PT66-Eu-Chelate to the streptavidine-XLent. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

Fyn Kinase Assay

C-terminally His6-tagged human recombinant kinase domain of the human T-Fyn expressed in baculovirus infected insect cells (purchased from Invitrogen, P3042) is used as kinase. As substrate for the kinase reaction the biotinylated peptide biotin-KVEKIGEGTYGVV (C-terminus in amid form) is used which can be purchased e.g. form the company Biosynthan GmbH (Berlin-Buch, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of T-Fyn in aqueous assay buffer [25 mM Tris/HCl pH 7.2, 25 mM MgCl₂, 2 mM dithiothreitol, 0.1% (w/v) bovine serum albumin, 0.03% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2 μM=>final conc. in the 5 μL assay volume is 1.2 μM) in assay buffer and the resulting mixture is incubated for a reaction time of 60 min at 22° C. The concentration of Fyn is adjusted depending of the activity of the enzyme lot and is chosen appropriate to have the assay in the linear range, typical concentration was 0.13 nM. The reaction is stopped by the addition of 5 μL of a solution of HTRF detection reagents (0.2 μM streptavidine-XL [Cisbio Bioassays, Codolet, France) and 0.66 nM PT66-Eu-Chelate, an europium-chelate labelled anti-phospho-tyrosine antibody from Perkin Elmer [instead of the PT66-Eu-chelate PT66-Tb-Cryptate from Cisbio Bioassays can also be used]) in an aqueous EDTA-solution (125 mM EDTA, 0.2% (w/v) bovine serum albumin in 50 mM HEPES/NaOH pH 7.0).

The resulting mixture is incubated 1 h at 22° C. to allow the binding of the biotinylated phosphorylated peptide to the streptavidine-XL and the PT66-Eu-Chelate. Subsequently the amount of phosphorylated substrate is evaluated by measurement of the resonance energy transfer from the PT66-Eu-Chelate to the streptavidine-XL. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer).

The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compounds are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

Flt4 Kinase Assay

Flt4 inhibitory activity of compounds of the present invention can be quantified employing the Flt4 TR-FRET assay as described in the following paragraphs.

As kinase, a GST-His fusion protein containing a C-terminal fragment of human Flt4 (amino acids 799-1298, expressed in insect cells [SF9] and purified by affinity chromatography, purchased from Proqinase [Freiburg i.Brsg., Germany] is used. As substrate for the kinase reaction the biotinylated peptide Biotin-Ahx-GGEEEEYFELVKKKK (C-terminus in amide form, purchased from Biosyntan, Berlin-Buch, Germany) is used.

For the assay 50 nL of a 100 fold concentrated solution of the test compound in DMSO was pipetted into a black low volume 384 well microtiter plate (Greiner Bio-One, Frickenhausen, Germany), 2 μL of a solution of Flt4 in aqueous assay buffer [25 mM HEPES pH 7.5, 10 mM MgCl₂, 2 mM dithiothreitol, 0.01% (v/v) Triton-X100 (Sigma), 0.5 mM EGTA, and 5 mM β-phospho-glycerol] are added and the mixture is incubated for 15 min at 22° C. to allow pre-binding of the test compounds to the enzyme before the start of the kinase reaction.

Then the kinase reaction is started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture is incubated for a reaction time of 45 min at 22° C. The concentration of Flt4 in the assay is adjusted depending of the activity of the enzyme lot and was chosen appropriate to have the assay in the linear range, typical enzyme concentrations are in the range of about 120 pg/μL (final conc. in the 5 μL assay volume). The reaction is stopped by the addition of 5 μL of a solution of HTRF detection reagents (200 nM streptavidine-XL665 [Cis Biointernational] and 1 nM PT66-Tb-Cryptate, an terbium-cryptate labelled anti-phospho-tyrosine antibody from Cisbio Bioassays (Codolet, France) in an aqueous EDTA-solution (50 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Tb-Cryptate. Subsequently the amount of phosphorylated substrate is evaluated by measurement of the resonance energy transfer from the PT66-Tb-Cryptate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

TrkA Kinase Assay

TrkA inhibitory activity of compounds of the present invention can be quantified employing the TrkA HTRF assay as described in the following paragraphs.

As kinase, a GST-His fusion protein containing a C-terminal fragment of human TrkA (amino acids 443-796, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GT0BLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of TrkA in aqueous assay buffer [8 mM MOPS/HCl pH 7.0, 10 mM MgCl₂, 1 mM dithiothreitol, 0.01% (v/v) NP-40 (Sigma), 0.2 mM EDTA] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [˜30 nM]) in assay buffer and the resulting mixture is incubated for a reaction time of 60 min at 22° C. The concentration of TrkA in the assay is adjusted depending of the activity of the enzyme lot and is chosen appropriate to have the assay in the linear range, typical enzyme concentrations are in the range of about 20 pg/μL (final conc. in the 5 μL assay volume). The reaction is stopped by the addition of 5 μL of a solution of HTRF detection reagents (30 nM streptavidine-XL665 [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the PT66-Eu-Chelate to the streptavidine-XL665. Therefore, the fluorescence emissions at 620 nm and 665 nm after excitation at 350 nm is measured in a HTRF reader, e.g. a Rubystar (BMG Labtechnologies, Offenburg, Germany) or a Viewlux (Perkin-Elmer). The ratio of the emissions at 665 nm and at 622 nm is taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

AlphaScreen SureFire eIF4E Ser209 Phosphorylation Assay The AlphaScreen SureFire eIF4E Ser209 phoshorylation assay can be used to measure the phosphorylation of endogenous eIF4E in cellular lysates. The AlphaScreen SureFire technology allows the detection of phosphorylated proteins in cellular lysates. In this assay, sandwich antibody complexes, which are only formed in the presence of the analyte (p-eIF4E Ser209), are captured by AlphaScreen donor and acceptor beads, bringing them into close proximity. The excitation of the donor bead provokes the release of singlet oxygen molecules that triggers a cascade of energy transfer in the Acceptor beads, resulting in the emission of light at 520-620 nm.

Surefire EIF4e Alphascreen in A549 Cells with 20% FCS Stimulation

For the assay the AlphaScreen SureFire p-eIF4E Ser209 10K Assay Kit and the AlphaScreen Protein A Kit (for 10K assay points) both from Perkin Elmer are used.

On day one 50.000 A549 cells are plated in a 96-well plate in 100 μL per well in growth medium (DMEM/Hams' F12 with stable Glutamin, 10% FCS) and incubated at 37° C. After attachment of the cells, medium is changed to starving medium (DMEM, 0.1% FCS, without Glucose, with Glutamin, supplemented with 5 g/L Maltose). On day two, test compounds are serially diluted in 50 μL starving medium with a final DMSO concentration of 1% and are added to A549 cells in test plates at a final concentration range from as high 10 μM to as low 10 nM depending on the activities of the tested compounds. Treated cells are incubated at 37° C. for 2 h. 37 ul FCS is added to the wells (=final FCS concentration 20%) for 20 min. Then medium is removed and cells are lysed by adding 50 μL lysis buffer. Plates are then agitated on a plate shaker for 10 min. After 10 min lysis time, 4 μL of the lysate is transferred to a 384 well plate (Proxiplate from Perkin Elmer) and 5 μL Reaction Buffer plus Activation Buffer mix containing AlphaScreen Acceptor beads is added. Plates are sealed with TopSeal-A adhesive film, gently agitated on a plate shaker for 2 hours at room temperature. Afterwards 2 μL Dilution buffer with AlphaScreen Donor beads are added under subdued light and plates are sealed again with TopSeal-A adhesive film and covered with foil. Incubation takes place for further 2 h gently agitation at room temperature. Plates are then measured in an EnVision reader (Perkin Elmer) with the AlphaScreen program. Each data point (compound dilution) is measured as triplicate.

Proliferation Assays

The tumor cell proliferation assay which can be used to test the compounds of the present invention involves a readout called Cell Titer-Glow® Luminescent Cell Viability Assay developed by Promega® (B. A. Cunningham, “A Growing Issue: Cell Proliferation Assays, Modern kits ease quantification of cell growth”, The Scientist 2001, 15(13), 26; S. P. Crouch et al., “The use of ATP bioluminescence as a measure of cell proliferation and cytotoxicity”, Journal of Immunological Methods 1993, 160, 81-88), that measures inhibition of cell proliferation. Generation of a luminescent signal corresponds to the amount of ATP present, which is directly proportional to the number of metabolically active (proliferating) cells.

In Vitro Tumor Cell Proliferation Assay:

Cultivated tumour cells (MOLM-13 (human acute myeloid leukemia cells obtained from DSMZ # ACC 554), JJN-3 (human plasma cell leukemia cells obtained from DSMZ # ACC 541), Ramos (RA1) (human Burkitt's lymphoma cells obtained from ATCC # CRL-159)) are plated at a density of 2,500 cells/well (JJN-3), 3,000 cells/well (MOLM-13), 4,000 cells/well (Ramos (RA1)), in a 96-well multititer plate (Costar 3603 black/clear bottom) in 100 μL of their respective growth medium supplemented with 10% fetal calf serum. After 24 hours, the cells of one plate (zero-point plate) are measured for viability. Therefore, 70 μL/well CTG solution (Promega Cell Titer Glo solution (catalog # G755B and G756B)) is added to zero-point plate. The plates are mixed for two minutes on orbital shaker to ensure cell lysis and incubated for ten minutes at room temperature in the dark to stabilize luminescence signal. The samples are read on a VICTOR 3 plate reader. In parallel, serially test compounds are diluted in growth medium, and 50 μL of 3× dilutions/well are pipetted into the test plates (final concentrations: 0 μM, as well as in the range of 0.001-30 μM). The final concentration of the solvent dimethyl sulfoxide is 0.3-0.4%. The cells are incubated for 3 days in the presence of test substances. 105 μL/well CTG solution (Promega Cell Titer Glo solution (catalog #G755B and G756B)) is added to the test wells. The plates are mixed for 2 minutes on an orbital shaker to ensure cell lysis and incubated for 10 min at room temperature in the dark to stabilize luminescence signal. The samples are read on a VICTOR 3 plate reader. The change of cell number, in percent, is calculated by normalization of the measured values to the extinction values of the zero-point plate (=0%) and the extinction of the untreated (0 μm) cells (=100%).

Overview cell lines for proliferation assays

| | | Cell | |
|---|---|---|---|
| Cell line | Origin | number/well | Culture Medium |
| MOLM-13 | human acute | 3000 | RPMI 1640 with |
| (obtained | myeloid | | stable Glutamin |
| from DSMZ # | leukemia | | with 10% Fetal |
| ACC 554) | | | Bovine Serum |
| JJN-3 | human | 2500 | 45% Dulbecco's |
| (obtained | plasma cell | | Modified Eagle |
| from DSMZ # | leukemia | | Medium with stable |
| ACC 541) | | | Glutamin, 45% |
| | | | Iscove's Modified |
| | | | Dulbecco's Media |
| | | | with stable Glutamin |
| | | | and 10% Fetal Bovine |
| | | | Serum |
| Ramos (RA1) | human | 4000 | RPMI 1640 media with |
| (obtained | Burkitt's | | stable Glutamin with |
| from ATCC # | lymphoma | | 10% Fetal Bovine Serum |
| CRL-159) | | | |

Thus the compounds of the present invention effectively inhibit one or more kinases and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In Vitro IL-1β (Interleukin-1 Beta, IL-b)-Induced Cytokine Secretion of Human PBMCs (Peripheral Blood Mononuclear Cells)

The effect of the chemical compounds on the induced cytokine secretion of human PBMCs has been investigated. Here, the cytokine secretion has been induced by IL-13 which binding to its receptors leads to the activation of the MKNK signaling pathway.

Human PBMCs have been isolated from anti-coagulated human whole blood donated from healthy volunteers by pre-filling Leucosep tubes with ficoll-paque (15 ml, Biochrom, order ID: L6115) and adding 20 ml whole blood. After centrifugation of the blood at 800 g for 15 min at room temperature (RT) plasma including thrombocytes has been discarded. PBMCs were transferred to a new falcon tube, washed with PBS (phosphate-buffered saline) at 250 g for 10 min at RT and resuspended in complete medium [RPMI 1640, without L-glutamine (PAA, order ID: E15-039), 10% FCS; 50 U/ml Penicillin, 50 μg/ml Streptomycin (PAA, order ID: P11-010) and 1% L-glutamine (Sigma, order ID. G7513)]. The assay was performed in a 96-well plate at a cell density of 2.5×10⁵ cells/well as triplicates. The compounds were serially diluted in DMSO and added to the PBMCs with a final concentration of 0.4% in DMSO, respectively. Treatment of PBMCs with 0.4% DMSO was used as control. After 30 min of incubation, PBMCs were stimulated with 20 ng/ml IL-1β (R&D, order ID: 201-LB CF) for 24 hours. Cell viability was measured using the CellTiter-Glo Luminescent Assay (Promega, order ID: G7571) following the manufacturers protocol. The amount of secreted IL-2, IL-6, IL-8, IL-10, IL-12p70, GM-CSF, IFN-γ, and TNF-α (tumor necrosis factor-alpha) in the supernatant was determined using the Human Prolnflammatory 9-Plex (MSD, order ID: K15007B) according to manufacturer's instruction. The inhibitory activity was determined as the relation to the control in percent. IC₅₀ values were calculated.

In Vitro LPS (Lipopolysaccharide)-Induced Cytokine Secretion of Human PBMCs (Peripheral Blood Mononuclear Cells)

The effect of chemical compounds on the induced cytokine secretion of human PBMCs has been investigated. Here, the cytokine secretion has been induced by IL-1β which binding to its receptor leads to the activation of the MKNK signaling pathway.

Human PBMCs have been isolated from anti-coagulated human whole blood donated from healthy volunteers by pre-filling Leukosep tubes with ficoll-paque (15 ml, Biochrom, order ID: L6115) and adding 20 ml whole blood. After centrifugation of the whole blood at 800 g for 15 min at room temperature (RT) plasma including thrombocytes has been discarded. PBMCs were transferred to a new falcon tube, washed with PBS (phosphate-buffered saline) at 250 g for 10 min at RT and resuspended in complete medium [RPMI 1640, without L-glutamine (PAA, order ID: E15-039), 10% FCS, 50 U/ml Penicillin, 50 μg/ml Streptomycin (PAA, order ID: P11-010) and 1% L-glutamine (Sigma, order ID: G7513)]. The assays were performed in a 96-well plate at a cell density of 2.5×10⁵ cells/well as triplicates. The compounds were serially diluted in DMSO and added to the PBMCs with a final concentration with 0.4% in DMSO, respectively. Treatment of PBMCs with 0.4% DMSO was used as a control. After 30 min of incubation, PBMCs were stimulated with 100 ng/ml LPS (Lipopolysaccharide, Sigma order ID: L4516) for 24 hours. Cell viability was measured using CellTiter-Glo Luminescent Assay (Promega, order ID: G7571) following the manufacturers protocol. The amount of secreted IL-1β, IL-2, IL-6, IL-8, IL-10, IL-12p70, IFN-γ, and TNF-α in the supernatant was determined using Human Prolnflammatory 9-Plex (MSD, order ID. K15007B) or IL-1β, IL-1ra, IL-2, IL-4, IL-5, IL-6, IL-7, IL-8, IL-9, IL-10, IL-12p70, IL-13, IL-15, IL-17, eotaxin, basic FGF, G-CSF, GM-CSF, IFN-γ, IP-10, MCP-1, MIP-1α, MIP-1β, PDGF-BB, RANTES, TNF-α, and VEGF using Bio-Plex Human Group I Assay (Bio-Rad, order ID: M500KCAFOY) according to manufacturer's instruction. The inhibitory activity was determined as the relation to the control in percent. IC₅₀ values were calculated.

In Vitro PHA (Phytohaemagglutinin)-Induced Cytokine Secretion of Human PBMCs (Peripheral Blood Mononuclear Cells)

The effect of chemical compounds on the induced cytokine secretion of human PBMCs has been investigated. Here, the cytokine secretion has been induced by IL-1β which binding to its receptor leads to the activation of the MKNK signaling pathway.

Human PBMCs have been isolated from anti-coagulated human whole blood donated from healthy volunteers by pre-filling Leukosep tubes with ficoll-paque (15 ml, Biochrom, order ID: L6115) and adding 20 ml whole blood. After centrifugation of the whole blood at 800 g for 15 min at room temperature (RT) plasma including thrombocytes has been discarded. PBMCs were transferred to a new falcon tube, washed with PBS (phosphate-buffered saline) at 250 g for 10 min at RT and resuspended in complete medium [RPMI 1640, without L-glutamine (PAA, order ID: E15-039), 10% FCS, 50 U/ml Penicillin, 50 μg/ml Streptomycin (PAA, order ID: P11-010) and 1% L-glutamine (Sigma, order ID: G7513)]. The assays were performed in a 96-well plate at a cell density of 2.5×10⁵ cells/well as triplicates. The compounds were serially diluted in DMSO and added to the PBMCs with a final concentration with 0.4% in DMSO, respectively. Treatment of PBMCs with 0.4% DMSO was used as a control. After 30 min of incubation, PBMCs were stimulated with 10 mg/ml PHA (Phytohaemagglutinine, Sigma, order ID: L4144) for 24 hours. Cell viability was measured using CellTiter-Glo Luminescent Assay (Promega, order ID: G7571) following the manufacturers protocol. The amount of secreted pro-inflammatory cytokines (e.g. IL-1β, IL-4, IL-6, IL-10, IL-17A, IL-17F, IL-21, IL-22, IL-23, IL-25, IL-31, IL-33, CD40L, TNF-α, IFN-γ) in the supernatant was determined using Bio-Plex Human Assay (Bio-Rad, order ID: 171AA001M) according to manufacturer's instruction. The inhibitory activity was determined as the relation to the control in percent. IC₅₀ values were calculated.

In Vitro—3T3-L1 Differentiation into Adipocyte-Like Cells

The effect of chemical compounds on the differentiation of pre-adipocytes into adipocytes has been investigated. Here, 3T3-L1 fibroblasts were differentiated into adipocyte-like cells.

Preadipocytes (3T3-L1) were grown to 2 days post-confluence in DMEM supplemented with 10% BCS (day 0) and the medium was changed to DMEM supplemented with 10% FBS, insulin (167 nM), dexamethasone (0.5 μM), isobutylmethylxanthine (IBMX) (0.5 mM) and rosiglitazone (2 μM) and test compounds. After 48 h, the medium replaced by DMEM supplemented with 10% (v/v) FBS and 167 nM insulin and different concentrations of test compound. This maintenance medium was changed every 48 h always containing different concentrations of the test compound. Cells underwent the differentiation program up to 9 days. Gene expression of MNK1, MNK2 as well as of adipocyte differentiation marker such as PPARγ, C/EBPα, SREBP1c, GLUT4, CD36, FAS, cytosolic phospholipase A2a was performed. The relative mRNA expression of each gene in compound treated cells was compared to the relative mRNA expression in differentiated but untreated control cells.

Oil Red O staining was used to qualify and quantify intracellular triglyceride levels. For quantification, cells were washed extensively with water to remove unbound dye, and 1 ml isopropanol was added to the stained cells. Photometric evaluation was applied.

Lipid incorporation was also measured by triglyceride assay and lipolysis assay.

Lipolysis describes the hydrolysis of triglycerides into glycerol and free fatty acids. Lipolysis is induced by the synthetic catecholamine, Isoproterenol and glycerol serves as a measure for the incorporated fat during differentiation into adipocytes. For the lipolysis assay, 3T3-L1 cells were differentiated, as described above, for 9 days. The lipolysis assay was performed according to the manufacturer's instructions (e.g. Abcam Lipolysis assay kit, ab185433). The amount of glycerol released was measured using colorimetric intensity.

In Vivo CFA (Complete Freund's Adjuvant)-Induced Inflammatory Pain in Rats

The complete Freund's adjuvant (CFA)-induced inflammatory pain model can be used to evaluate the effect of the chemical compounds on pain.

On day 0, rats (Sprague-Dawley, 10 animals/group) receive either vehicle or the chemical compound before injection of CFA followed by daily treatment until day 7. To inject 100 μl of 100% CFA (Sigma, order ID: F5881) subcutaneously into the plantar surface of the left hind paw rats are anesthetized with 2.5-5% isoflurane in oxygen. Mechanical allodynia is assessed using von Frey filaments according to the “up-down” method described by Chaplan et al (1994) on day 7. Here, calibrated monofilaments (von Frey filaments) are applied to the plantar surface of the rat hind paw for a period of 4-6 seconds, or until a nocifensive paw withdrawal occurs.

In Vivo Imiquimod-Induced Psoriasis Model in Mice

Chemical compounds were tested for their effects in an Imiquimod (IMQ)-induced psoriasis model in mice.

Induction of psoriasis was performed on animals by daily administration of Imiquimod after shaving and depilation of the animals at Day −1. 3.5 mg of IMQ (equivalent to 70 mg of Aldara® creme, Meda AB) was topically administered from Day 1 to Day 7 (seven days) in the morning for groups to be induced. Sham group was applied with paraffin oil. The surface of application was about 4 cm² on the back of the animals which corresponds to a rectangle of about 1.5 cm to 2.5 cm on the back of the animal. The cream was smoothly massaged on the back for 5 seconds. After this time, the internal face of the right ear was massaged with remaining cream from finger glove. Then, the back was massaged again for 5 seconds and afterwards the internal face of the left ear was massaged with the remaining cream from finger gloves. Animals were weighed every second days (Day 1 prior induction, Day 3, Day 5 and Day 7). Due to body weight loss observed with this model, 0.4 ml of sterile saline was injected intraperitoneally (ip) at Day 3 and Day 4. Oral (po) administration of test compounds started at Day 1 not more than 1 hour after IMQ treatment. The test compounds was administered once daily from Day 1 to Day 7. In each treatment group n=10 animals were included with a Sham (no IMQ) group, a negative control group (IMQ plus vehicle), two groups treated with IMQ and different dosages of the test compound, and one group with IMQ treated animals receiving the reference (betamethasone) compound. Development of the disease was determined using a clinical scoring system. Every day, the following clinical scores were recorded from back skin Score Erythema Scaling Skin thickness

| Score | Erythema | Scaling | Skin thickness |
|---|---|---|---|
| 0 | Normal | No | Normal |
| 1 | Slight | Slight | Slight |
| 2 | Moderate | Moderate | Moderate |
| 3 | Important | Important | Important |
| 4 | Very important | Very important | Very important |

Both ears and back skin thickness was measured daily using digital sliding caliper (Horex Digital Caliper, Helios Preisser, Germany). At Day 4, 4 hours after morning treatment, blood was collected from the mandibular vein under isofluran anesthesia into heparin sodium tubes (0.5 ml Eppendorf tubes). After blood collection, 0.4 ml of sterile saline was injected ip to the animals. At Day 7 about 500 μl blood were collected after intercardiac puncture from isofluran-anesthetized in to heparin sodium tubes. Blood was centrifuged (3500 g for 10 min at 5° C. Plasma was separated and stored at −80° C. for further analyses (determination of cytokine levels). At Day 7 animals were euthanized, and back skin calibrated digital punctures were taken. Skin samples from the back and both ears were kept in formalin and placed in 70% ethanol 48 hours after collection. Histopathological evaluation of back and ear skin was done after hematoxylin and eosin (HE) staining by an experienced pathologist.

In Vivo MOG₃₃₋₃₅-Induced, Chronic EAE (Experimental Autoimmune Encephalomyelitis)-Model in Mice

The effects of chemical compounds were evaluated in an experimental autoimmune encephalomyelitis (EAE) model in C57Bl/6 mice.

On Day 0 all mice, except the animals from healthy control group were given a subcutaneous (sc) injection of 0.1 ml on two sites on the back of 200 μg MOG₃₅₋₅₅ (myelin oligodendrocyte glycoprotein) emulsified in Complete Freund's Adjuvant supplemented with Mycobacterium tuberculosis. At this same time point, mice were injected with 200 ng of Pertussis Toxin (PT) dissolved in 0.1 ml PBS (phosphate buffered saline); the PT injection was repeated on Day2. A group of ten animals served as naïve, vehicle-dosed control group. The other groups underwent induction of EAE and received test compound treatment. Mice were weighed daily. Symptoms of EAE were assessed daily, starting on Day 4 and continuing through study end. All remaining mice were sacrificed at the completion of the study (Day 30). Blood was collected for preparation of serum. Animals were then perfused with formalin, and the spinal cord was collected and stored in 10% formalin for subsequent histopathology analysis. Slides were evaluated by a broad certified veterinary pathologist and were assessed for inflammation and demyelination on a 0-5 scoring scale. Terminal serum samples were analyzed via multiplex analyses for levels of TNF-α, IL-6, IL-12, IL-23, IL-1β, IL-17, and IFNγ.

In Vivo Adjuvant-Induced Arthritis in Rats

The effects of chemical compounds on the adjuvant-induced arthritis in rats were investigated.

At day 0 male Lewis rats (100 to 125 g body weight, Charles River Laboratories, Germany) were treated at the tail subcutaneously (sc) with 100 μl of complete Freund's Adjuvant (CFA) solution [M. tuberculosis H37Ra (Difco Lab, cat. No. 231141)] diluted in Incomplete Freund's Adjuvant (Difco Lab, cat. no: 263910). Animals were randomized with n=8 animals per treatment group. As controls a healthy and a disease group treated with vehicle only were included in the studies. Treatment with test compounds was done orally (po) with either one or more dosages using an appropriate vehicle which allowed sufficient exposure of the animals with the test compound. In a preventive treatment setting, treatment start was at day 0 and continued to day 20, the end of the study. Observation of the disease induction as well as the treatment effects of test compound was determined by the RA disease activity score, starting at day 0 and then three times per week. The Score defines the extent of joint inflammation from 0 to 4 including erythema with swelling of the joint (0=no; 1=slight; 2=moderate, 3=important, 4=very important). The disease score was determined for both hind paws and added to one value. As an additional parameter for joint swelling the paw volume was determined using a plethysometer (IITC Life Science Inc., USA). A second parameter determined during the study was the grip strength using an automated grip strength test meter (IITC Life Science Inc., USA) as a measure for hyperalgesia. At the end of the study (day 20) synovial fluid from joints, biopsies of kneejoint and blood serum were obtained and used for determination of proinflammatory cytokines [Meso Scale Discovery (MSD), Prolnflammatory Panel 1; cat no: K15059D), and the c-reactive protein (CRP) (BD Biosciences, cat no: 55825).

Synovial fluid is isolated by rinsing the inflamed joints with 150 μl sterile sodium acetate solution. Biopsies of kneejoints were homogenized with a cryo mill (Retsch GmbH, Germany) at −196° C. 200 mg of the powder were used for analysis of cytokines suspended in 0.5 ml RPMI1640 medium. Statistical analyses of obtained results were done with one way Anova (ANOVA; Analysis of variance) and the comparison to the control group via multiple reference analysis (Dunnett-test).

In Vivo—Fertility Model in Mice Chemical compound were tested for their effects on fertility in a DBA/2J-CBA/J mouse model. Female CBA/J mice bred with mal DBA/2J display a higher abortion rate.

Male DBA/2J mice were bred with female CBA/J mice and the vaginal plugs in individual mated female mice were examined daily to determine potential pregnancy. At Day 1 of pregnancy one or two groups of successfully mated female CBA/J mice were treated orally (po) with one or two different dosages of the test compound once daily, whereas the control group was given the vehicle only. In each treatment group n=12 animals were included. At Day 10 to Day 14 of pregnancy the animals were sacrificed, the uteri removed and the implantation sites were documented. The abortion sites were identified by their small size and necrotic, hemorrhagic appearance, compared with normal embryos and placentas. The percentage of abortions was calculated as the ratio of resorption sites to total implantation sites. Uteri were shock frozen and after homogenization cytokine levels were determined using mouse Bio-Plex Assays (M0009RDPD, M6000007NY, LJ00000163).

In Vivo CCL4-Induced Liver Fibrosis in Mice

The effect of chemical compounds was tested in a tetrachlormethan (CCL4)-induced mouse model of liver fibrosis.

Eight week old 90 male C57Bl/6 mice (Charles River) were randomly divided into three groups (Group 1=untreated control, Group 2=vehicle treated control, Group 3=treatment with test compound) with n=30 in each group. For induction of liver fibrosis animals were treated three times per week (Monday, Wednesday, Friday) with 50 μl of CCL4/olive oil suspension (CCL4+olive oil, 1+9 v/v) intraperitoneally (ip) over the whole study time. CCL4 is the most commonly used inducer of a toxically-induced liver fibrosis in animal models (Starkel et al., Animal models for the study of hepatic fibrosis. Best Practice & Research Clinical Gastroenterology 25 (2011) 319-333). Once daily per os (po) treatment of group 2 with the vehicle and of group 3 with test compound suspension started with the first day and was done over the complete study duration. Two weeks after study start fifty percent of animals (of each group) were euthanized and after two additional weeks, the remaining animals were euthanized. After finalization of the study, the liver of each animal as collected and fixed in 4% formaldehyde and paraffin embedded for further histopathological analyses. For determination of severity of liver fibrosis liver slices were stained with pikro-sirius red (Waldeck, Germany) to visualize the collagen content in the tissue. A Carl Zeiss microscope (Axio) connected to a PC was used to scan the pikro-sirius red stained liver sections to make images of these. The sections were scanned at a magnification of 20× and a light intensity of 4.8V. The images were then formatted into jpg and the red-stained area quantified by using the ImageJ Software (National Institue of Health, USA). The results are expressed as % sirius-red per liver area.

Chemical compounds of the invention are tested for their effects in following in vivo models.

In Vivo—Letrozole-Induced Polycystic Ovary Syndrome in Rats

Chemical compounds are tested for their effects in a Polycystic Ovary Syndrome (PCOS) model in rats.

Han Wistar rats are randomly divided into 3 groups with n=8 animals per group. Rats in the control group receive vehicle only once daily per os (po), whereas rats of the other two groups are all administered letrozole at a concentration of 1 mg/kg body weight dissolved in 0.5% carboxymethylcellulose (CMC) once daily for consecutive 28 days. Animals of the third group additionally receive the test compound once daily po in 20% HPβCD (HP beta cyclodextrin) for consecutive 28 days. Vaginal smears are performed, and the rat weights are recorded daily. At day 27 animals are fasted and an oral glucose tolerance test will be performed. Rats are euthanized, ovaries are removed and weighed. One ovary of each rat is fixed in formaldehyde for histological examination, whereas the other ovary is stored at −80° C. for mRNA and protein analyses. Fat tissue and liver are also removed and from each tissue one aliquot is fixed in formaldehyde and the other aliquot is shock frozen for further mRNA analyses.

Vaginal Smears: the stage of the estrous cycle is determined by microscopic analysis of the predominant cell type in the daily vaginal smears.

Ovarian morphology: sections from the ovarian tissue are taken from the part of the ovary with largest diameter, stained with hematoxylin and eosin.

In Vivo—Diet Induced Obesity in Mice

Effects of chemical compound on diet-induced obesity are tested in C57Bl/6 mice receiving a high fat diet.

Male C57Bl/6 (n=45) mice at an age of 16 weeks are randomly divided into 5 groups. Animals fo groups 1 to 4 are fed with 60% high fat diet for 10 weeks. Animals of group 5 (n=10) are fed with chow diet for the same time. Mice of group 1 are additionally treated orally (BID) with vehicle only. Animals of group 2 (n=15) receive test compound (BID) orally. Mice of group 3 (n=10) receive pioglitazone via oral administration (QD). Compound and vehicle treatment is performed over the complete study duration. Animals of groups 4 (n=10) and 5 (n=10) do not receive any treatment. Body weight is measured daily during the 8 weeks treatment period. At days 26 and 48 of treatment overnight fasted mice undergo an oral glucose tolerance test (OGTT). Mice are weighed and blood (15 μl/EDTA) is collected from the tail tip two hours after compound treatment; 30 min prior glucose load to measure HBA1c, blood glucose, and plasma insulin and HOMAR-IR will then be calculated.

HOMAR-IR index=[(mM glucose X μU/ml insulin)/22.5]

For OGTT blood glucose is measured from tail tip at −30, 0, 15, 30, 60, 90, and 120 min after oral glucose load (1.5 g/kg body weight). Blood (20 μl/EDTA) is also collected at 15 and 30 min after glucose load to measure plasma insulin. Blood glucose levels are used to calculate glucose area under the curve. Mice recover from the OGTT over 2 days.

At days 28 of treatment blood (15 μl/EDTA) is collected 2 hours after compound treatment from the tail tip to measure HBA1c, blood glucose and plasma insulin in fed conditions and HOMAR-IR will then be calculated.

At day 51 of treatment 4 h fasted mice then undergo an Insulin Tolerance Test (ITT) with insulin (0.5 U/kg body weight) injected intraperitoneally. Blood glucose is then measured at time 0, 15, 30, 60, 90, and 120 minutes after insulin injection.

At day 53 of treatment the incorporated body fat is determined by Echo-MRI. At day 56 of treatment (end of the study) blood is taken for determination of blood glucose, leptin, triglycerides, total cholesterol, and free fatty acids.

In Vivo Pharmacokinetics in Rats

For in vivo pharmacokinetic experiments test compounds were administered to male Wistar rats intravenously at doses of 0.3 to 1 mg/kg and intragastral at doses of 0.5 to 10 mg/kg formulated as solutions using solubilizers such as PEG400 in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 6 h, 8 h and 24 h after dosing. Depending on the expected half-life additional samples were taken at later time points (e.g. 48 h, 72 h). For pharmacokinetics after intragastral administration test compounds were given intragastral to fasted rats and blood samples were taken at 5 min, 15 min, 30 min, 45 min, 1 h, 2 h, 4 h, 6 h, 8 h and 24 h after dosing.

Depending on the expected half-life additional samples were taken at later time points (e.g. 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitril and frozen at −20° C. over night. Samples were subsequently thawed and centrifuged at 3000 rpm, 4° C. for 20 minutes. Aliquots of the supernatants were taken for analytical testing using an Agilent 1200 HPLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0−tlast)norm: Area under the concentration-time curve from t=0 h to the last time point for which plasma concentrations could be measured divided by the administered dose (in kg*h/L); t1/2: terminal half-life (in h); F: oral bioavailability: AUCnorm after intragastral administration divided by AUCnorm after intravenous administration (in %).

1: A compound of formula (I) 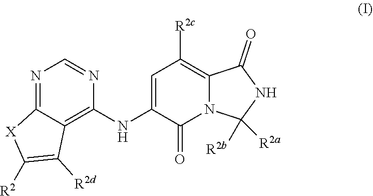
wherein
X represents NH, O or S;
R², R^{2d} are taken together to form a C₃-C₅-alkylene group said group being optionally substituted, identically or differently, with 1, 2, or 3 R⁵-groups selected from the group consisting of hydrogen, halogen, cyano, hydroxy, —C₁-C₃-alkyl, —C₁-C₃-alkyl-OH,
—C₁-C₆-alkoxy, —(C═O)—C₁-C₆-alkyl, —(C═O)—C₃-C₆-cycloalkyl, —C(C═O)—OH, —C(C═O)—O—C₁-C₆-alkyl, —NH₂, —N(H)—C₁-C₃-alkyl, —N(—C₁-C₃-alkyl)-C₁-C₃-alkyl, —(C═O)—N(H)—C₁-C₆-alkyl, —(C═O)—N(—C₁-C₆-alkyl)-C₁-C₆-alkyl, and —NH(C═O)—O—C₁-C₆-alkyl; or
said C₃-C₅-alkylene group is substituted one time with R¹ which is selected from the group consisting of a hydrogen atom, —CO₂—C₁-C₆-alkyl, —CO₂H, and —C(═O)N(R³)R⁴;
R^{2a}, R^{2b} represent independently from each other a group selected from the group consisting of hydrogen and C₁-C₆-alkyl-;
or
C(R^{2a})R^{2b} are together to form a group selected from the group consisting of C(═O), a 3- to 8-membered cycloalkyl- group, and a 4- to 8-membered heterocycloalkyl- group,
wherein said 3- to 8-membered cycloalkyl- or 4- to 8-membered heterocycloalkyl- group is optionally substituted one, two or three times, identically or differently, with a halogen atom or a group selected from the group consisting of —OR⁸,
—N(R⁸)R⁹, —CN, —C(═O)N(R⁸)R⁹, C₁-C₃-alkyl-, C₁-C₃-haloalkyl-, C₁-C₆-alkoxy-, C₁-C₆-haloalkoxy-, aryl, —(CH₂)_{q}-heteroaryl, and —(CH₂)_{q}—N(R⁸)R⁹;
R^{2c} is a hydrogen or a halogen atom or a C₁-C₄-alkyl- group;
wherein said a C₁-C₄-alkyl- group is optionally substituted one, two or three times, identically or differently, with a halogen atom or a group selected from the group consisting of —OR⁸ and —N(R⁷)R⁸;
R³ is a hydrogen atom or a C₁-C₆-alkyl- group;
wherein said C₁-C₆-alkyl- group is optionally substituted one, two or three times, identically or differently, with a group selected from the group consisting of halo-, C₁-C₃-alkoxy-, HO—, and —N(R⁸)R⁹;
R⁴ is a hydrogen atom or a C₁-C₄-alkyl- group;
wherein said C₁-C₄-alkyl- group is optionally substituted one, two or three times, identically or differently, with a halogen atom or a group selected from the group consisting of HO—, C₁-C₃-alkoxy-, —CN, —N(R⁸)R⁹, —N(R⁷)R⁸, —C(═O)N(R⁸)R⁹, and —C(═O)N(R⁷)R⁸;
or
N(R³)R⁴ are taken together to form a 3- to 10-membered heterocycloalkyl- or a 4- to 10-membered heterospirocycloalkyl group;
wherein said 3- to 10-membered heterocycloalkyl- group is optionally substituted one, two or three times, identically or differently, with a halogen atom or a group selected from the group consisting of C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, and —(C₁-C₃-alkyl)-N(R⁸)R⁹;
R⁷ is a C₁-C₄-alkyl- or a —C(═O)—O—C₁-C₆-alkyl group; wherein said C₁-C₄-alkyl- group is optionally substituted once with —OH or —N(R⁸)R⁹;
R⁸ is a hydrogen atom or a C₁-C₄-alkyl- group;
R⁹ is a hydrogen atom or a C₁-C₆-alkyl- group;
q is an integer selected from the group consisting of 0, 1, 2, and 3;
or a tautomer, an N-oxide, a hydrate, a solvate, or a salt thereof, or a mixture of any of the foregoing.

2: The compound according to claim 1, wherein the compound is a compound of formula (Ia) 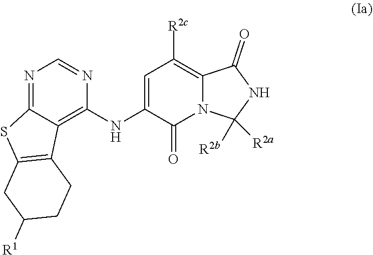
wherein
R¹ is selected from the group consisting of a hydrogen atom, CO₂—C₁-C₆-alkyl, —CO₂H, and —C(═O)N(R³)R⁴;
R^{2a}, R^{2b} are independently of each other a group selected from the group consisting of hydrogen and C₁-C₆-alkyl-; or
C(R^{2a})R^{2b} together form a group selected from the group consisting of C(═O), a 3- to 8-membered cycloalkyl- group, a 4- to 8-membered heterocycloalkyl- group,
wherein said 3- to 8-membered cycloalkyl-, 4- to 8-membered heterocycloalkyl-group is optionally substituted one, two or three times, identically or differently, with a halogen atom or a group selected from the group consisting of —OR⁸, —N(R⁸)R⁹, —CN, —C(═O)N(R⁸)R⁹, C₁-C₃-alkyl-, C₁-C₃-haloalkyl-, C₁-C₆-alkoxy-, C₁-C₆-haloalkoxy-, aryl, —(CH₂)_{q}-heteroaryl, and —(CH₂)_{q}—N(R⁸)R⁹;
R^{2c} is selected from the group consisting of a hydrogen atom, a halogen atom, and a C₁-C₄-alkyl- group; wherein said a C₁-C₄-alkyl- group is optionally substituted one, two or three times, identically or differently, with a halogen atom or a group selected from the group consisting of —OR⁸, and —N(R⁷)R⁸;
R³ is a hydrogen atom or a C₁-C₆-alkyl- group; wherein said C₁-C₆-alkyl- group is optionally substituted one, two or three times, identically or differently, with a group selected from the group consisting of halo-, C₁-C₃-alkoxy-, HO—, and —N(R⁸)R⁹;
R⁴ is a hydrogen atom or a C₁-C₄-alkyl- group; wherein said C₁-C₄-alkyl- group is optionally substituted one, two or three times, identically or differently, with a halogen atom or a group selected from the group consisting of HO—, C₁-C₃-alkoxy-, —CN, —N(R⁸)R⁹, —N(R⁷)R⁸, —C(═O)N(R⁸)R⁹, and —C(═O)N(R⁷)R⁸;
or
N(R³)R⁴ together form a 3- to 10-membered heterocycloalkyl- group or a 4- to 10-membered heterocycloalkenyl group; wherein said 3- to 10-membered heterocycloalkyl- group is optionally substituted one, two or three times, identically or differently, with a halogen atom or a group selected from the group consisting of C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, and —(C₁-C₃-alkyl)-N(R⁸)R⁹;
R⁷ is a C₁-C₄-alkyl- group or a —C(═O)—O—C₁-C₆-alkyl- group; wherein said C₁-C₄-alkyl- group is optionally substituted once with —OH or —N(R⁸)R⁹;
R⁸ is a hydrogen atom or a C₁-C₄-alkyl- group;
R⁹ is a hydrogen atom or a C₁-C₆-alkyl- group;
q is an integer selected from the group consisting of 0, 1, 2, and 3;
or a tautomer, an N-oxide, a hydrate, a solvate, or a salt thereof, or a mixture of any of the foregoing.

3: The compound according to claim 1, wherein the compound is a compound of formula (Ib): 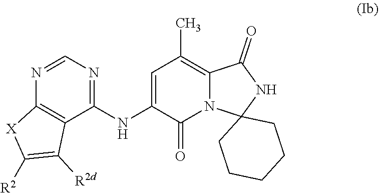
wherein
X is NH, O or S;
R², R^{2d} together form a C₃-C₅-alkylene group;
said group being optionally substituted, identically or differently, with 1, 2, or 3 R⁸-groups;
R⁸ is selected from the group consisting of hydrogen, halogen, cyano, hydroxy, —C₁-C₃-alkyl, —C₁-C₃-alkyl-OH, —C₁-C₆-alkoxy, —(C═O)—C₁-C₆-alkyl, —(C═O)—C₃-C₆-cycloalkyl, —C(C═O)—OH, —C(C═O)—O—C₁-C₆-alkyl, —NH₂, —N(H)—C₁-C₃-alkyl, —N(—C₁-C₃-alkyl)-C₁-C₃-alkyl, —(C═O)—N(H)—C₁-C₆-alkyl, —(C═O)—N(—C₁-C₆-alkyl)-C₁-C₆-alkyl, and —NH(C═O)—O—C₁-C₆-alkyl;
or a tautomer, an N-oxide, a hydrate, a solvate, or a salt thereof, or a mixture of any of the foregoing.

4: The compound according to claim 1, wherein the compound is of formula (Ia) 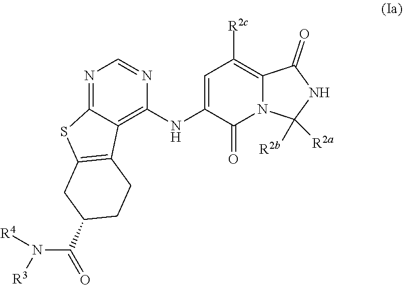
wherein
R^{2a}, R^{2b} are independently of each other a group selected from the group consisting of hydrogen, methyl, and fluoro; or
C(R^{2a})R^{2b} together form —(CH₂—CH_{2—})—, —(CH₂—CR₆(R₆ₐ)—CH_{2—})—, —(CH₂—Z—CH_{2—})—, —(CH₂—CH₂—CR₁₁(R₁₁ₐ)—CH_{2—})—, or —(CH₂—CH₂—CR₁₁(R₁₁ₐ)—CH₂—CH_{2—})—; and
Z is NR⁷, O, S, S(═O) or SO₂;
R^{2c} is a hydrogen, fluor, chlor or methyl;
R³ is a hydrogen atom or a C₁-C₆-alkyl- group; wherein said C₁-C₆-alkyl- group is optionally substituted one, two or three times, identically or differently, with a group selected from the group consisting of halo-, C₁-C₃-alkoxy-, HO—, and —N(R⁸)R⁹;
R⁴ is a hydrogen atom or a C₁-C₄-alkyl- group; wherein said C₁-C₄-alkyl- group is optionally substituted one, two or three times, identically or differently, with a halogen atom or a group selected from the group consisting of HO—, C₁-C₃-alkoxy-, —CN, —N(R⁸)R⁹, —N(R⁷)R⁸, —C(═O)N(R⁸)R⁹, and —C(═O)N(R⁷)R⁸;
or
N(R³)R⁴ together form a 3- to 10-membered heterocycloalkyl- group or a 4- to 10-membered heterocycloalkenyl group; wherein said 3- to 10-membered heterocycloalkyl- group is optionally substituted one, two or three times, identically or differently, with a halogen atom or a group selected from the group consisting of C₁-C₆-alkyl-, —(CH₂)_{q}—OH, —N(R⁷)R⁸, —N(R⁸)R⁹, C₁-C₃-alkyl-, —CN, C(═O)N(R⁸)R⁹, and —(C₁-C₃-alkyl)-N(R⁸)R⁹;
R⁶, R^{6a} are independently from one another a hydrogen atom, fluor or a C₁-C₄-alkyl- group;
R⁷ is a C₁-C₄-alkyl- group or a —C(═O)—O—C₁-C₆-alkyl- group; wherein said C₁-C₄-alkyl- group is optionally substituted once with —OH or —N(R⁸)R⁹;
R⁸ is a hydrogen atom or a C₁-C₄-alkyl- group;
R⁹ is a hydrogen atom or a C₁-C₆-alkyl- group;
q is an integer selected from the group consisting of 0, 1, 2, and 3;
or a tautomer, an N-oxide, a hydrate, a solvate, or a salt thereof, or a mixture of any of the foregoing.

5: The compound according to claim 1, wherein R^{2a}, R^{2b} are independently from each hydrogen or methyl, or a tautomer, an N-oxide, a hydrate, a solvate, or a salt thereof, or a mixture of any of the foregoing.

6: The compound according to claim 1, wherein C(R^{2a})R^{2b} together form —(CH₂—CH_{2—})—, —(CH₂—CH₂—CH_{2—})—, —(CH₂—S—CH_{2—})—, —(CH₂—S(O)₂—CH₂)—, —(CH₂—CH₂—CH₂—CH_{2—})—, —(CH₂—CH₂—CH₂—CH₂—CH_{2—})—, —(CH₂—CH₂—CF₂—CH₂—CH_{2—})—, —(CH₂—CH₂—C(CF₃)OH—CH₂—CH_{2—})—, or —(CH₂—CH₂—C(CF₂CF₃)OH—CH₂—CH_{2—})—, or a tautomer, an N-oxide, a hydrate, a solvate, or a salt thereof, or a mixture of any of the foregoing.

7: The compound according to claim 1, wherein R^{2c} is methyl, or a tautomer, an N-oxide, a hydrate, a solvate, or a salt thereof, or a mixture of any of the foregoing.

8: The compound according to claim 1, wherein R², R^{2d} together form —CH₂—CH(C(═O)OH)—CH₂—CH₂—, or —CH₂—CH(C(═O)NH₂)—CH₂—CH₂—, or a tautomer, an N-oxide, a hydrate, a solvate, or a salt thereof, or a mixture of any of the foregoing.

9: The compound according to claim 1, which is selected from the group consisting of
(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid,
(7S)—N,N-dimethyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide,
6′-{[(7S)-7-(azetidin-1-ylcarbonyl)-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione,
(7S)—N-methyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide,
1-({(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile,
6′-({(7S)-7-[(3-hydroxy-3-methylazetidin-1-yl)carbonyl]-5,6,7,8-tetrahydro[1]benzothieno[2,3-d]pyrimidin-4-yl}amino)-8′-methyl-2′H-spiro[cyclohexane-1,3′-imidazo[1,5-a]pyridine]-1′,5′-dione,
tert-butyl [1-({(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidin-3-yl]carbamate,
8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione,
6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione,
8′-methyl-6′-({(7S)-7-[(4-oxopiperidin-1-yl)carbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione,
6′-({(7S)-7-[(3-aminoazetidin-1-yl)carbonyl]-5,6,7,8-tetrahydro[1]benzothieno[2,3-d]pyrimidin-4-yl}amino)-8′-methyl-2′H-spiro[cyclohexane-1,3′-imidazo[1,5-a]pyridine]-1′,5′-dione,
(7S)—N-[2-(dimethylamino)ethyl]-N-methyl-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide,
8′-methyl-6′-({(7S)-7-[(3-oxo-8-azabicyclo[3.2.1]oct-8-yl)carbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione,
6′-{[(7S)-7-{[(3S)-3,4-dimethylpiperazin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione,
(7S)-4-[(3,3,8-Trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid,
(7S)-4-[(8′-Methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid,
(7S)-4-[(8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide,
8′-methyl-6′-{[(7S)-7-{[(3R)-3-methylmorpholin-4-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione,
6′-{[(7S)-7-{[3-(dimethylamino)azetidin-1-yl]carbonyl}-5,6,7,8-tetrahydro[1]benzothieno[2,3-d]pyrimidin-4-yl]amino}-8′-methyl-2′H-spiro[cyclohexane-1,3′-imidazo[1,5-a]pyridine]-1′,5′-dione,
(7S)—N,N-dimethyl-4-[(8′-methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxamide,
6′-{[(7S)-7-(azetidin-1-ylcarbonyl)-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione,
1-({(7S)-4-[(8′-methyl-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile,
6′-{[(7S)-7-{[3-(dimethylamino)azetidin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione,
6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione,
6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-8′-methyl-2′H-spiro[cyclobutane-1,3′-imidazo[1,5-a]pyridine]-1′,5′-dione,
8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione,
(7S)—N,N-dimethyl-4-[(3,3,8-trimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxamide,
6-{[(7S)-7-(azetidin-1-ylcarbonyl)-5,6,7,8-tetrahydro [1]benzothieno [2,3-d]pyrimidin-4-yl]amino}-3,3,8-trimethyl-2,3-dihydroimidazo[1,5-a]pyridine-1,5-dione,
1-({(7S)-4-[(3,3,8-trimethyl-1,5-dioxo-1,2,3,5-tetrahydroimidazo[1,5-a]pyridin-6-yl)amino]-5,6,7,8-tetrahydro[1]benzothieno[2,3-d]pyrimidin-7-yl}carbonyl)azetidine-3-carbonitrile,
6-{[(7S)-7-{[3-(dimethylamino)azetidin-1-yl]carbonyl}-5,6,7,8-tetrahydro[1]benzothieno[2,3-d]pyrimidin-4-yl]amino}-3,3,8-trimethyl-2,3-dihydroimidazo[1,5-a]pyridine-1,5-dione,
6-{[(7S)-7-{[(3S)-3-(dimethylamino)pyrrolidin-1-yl]carbonyl}-5,6,7,8-tetrahydro [1]benzothieno[2,3-d]pyrimidin-4-yl]amino}-3,3,8-trimethyl-2,3-dihydroimidazo[1,5-a]pyridine-1,5-dione,
6-{[(7S)-7-{[(3R)-3,4-dimethylpiperazin-1-yl]carbonyl}-5,6,7,8-tetrahydro [1]benzothieno[2,3-d]pyrimidin-4-yl]amino}-3,3,8-trimethyl-2,3-dihydroimidazo[1,5-a]pyridine-1,5-dione,
3,3,8-trimethyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2,3-dihydroimidazo[1,5-a]pyridine-1,5-dione,
(7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid,
6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione,
8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione,
(7S)—N,N-dimethyl-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxamide,
6′-{[(7S)-7-(azetidin-1-ylcarbonyl)-5,6,7,8-tetrahydro[1]benzothieno[2,3-d]pyrimidin-4-yl]amino}-8′-methyl-2′H-spiro[cyclopentane-1,3′-imidazo [1,5-a]pyridine]-1′,5′-dione,
1-({(7S)-4-[(8′-methyl-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile,
6′-{[(7S)-7-{[3-(dimethylamino)azetidin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione,
6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-8′-methyl-2′H-spiro[cyclopentane-1,3′-imidazo[1,5-a]pyridine]-1′,5′-dione,
(7S)-4-[(4,4-Difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid,
8′-methyl-6′-(5,6,7,8-tetrahydro[1]benzothieno[2,3-d]pyrimidin-4-ylamino)-2′H-spiro[cyclohexane-1,3′-imidazo[1,5-a]pyridine]-1′,5′-dione,
(7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid,
(7S)—N,N-dimethyl-4-[(8-methyl-1,5-dioxo-1,5-dihydro-2H-spiro[imidazo [1,5-a]pyridine-3,3′-thietan]-6-yl)amino]-5,6,7,8-tetrahydro[1]benzothieno[2,3-d]pyrimidine-7-carboxamide,
6-{[(7S)-7-(azetidin-1-ylcarbonyl)-5,6,7,8-tetrahydro [1]benzothieno [2,3-d]pyrimidin-4-yl]amino}-8-methyl-2H-spiro[imidazo[1,5-a]pyridine-3,3′-thietane]-1,5-dione,
1-({(7S)-4-[(8-methyl-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidin-7-yl}carbonyl)azetidine-3-carbonitrile,
6-{[(7S)-7-{[3-(dimethylamino)azetidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione,
6-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione,
6-{[(7S)-7-{[(3R)-3,4-dimethylpiperazin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione,
8-methyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione,
(7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-N,N-dimethyl-5,6,7,8-tetrahydro[1]benzothieno[2,3-d]pyrimidine-7-carboxamide,
6′-{[(7S)-7-(azetidin-1-ylcarbonyl)-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione,
1-({(7S)-4-[(4,4-difluoro-8′-methyl-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile,
6′-{[(7S)-7-{[3-(dimethylamino)azetidin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione,
6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione,
6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-4,4-difluoro-8′-methyl-2′H-spiro[cyclohexane-1,3′-imidazo[1,5-a]pyridine]-1′,5′-dione,
4,4-difluoro-8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione,
(7S)-4-[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino 1-5,6,7,8-tetrahydro[1]benzothieno[2,3-d]pyrimidine-7-carboxylic acid,
(7S)-4-{[(cis)-4-hydroxy-8′-methyl-1′,5′-dioxo-4-(trifluoromethyl)-1′,5′-dihydro-2′H-spiro[cyclohexane-1,3′-imidazo [1,5-a]pyridin]-6′-yl]amino}-N,N-dimethyl-5,6,7,8-tetrahydro[1]benzothieno[2,3-d]pyrimidine-7-carboxamide,
(cis)-6′-{[(7S)-7-(azetidin-1-ylcarbonyl)-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione,
(cis)-6′-{[(7S)-7-{[3-(dimethylamino)-1,3-diazetidin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo [1,5-a]pyridine]-1′,5′-dione,
(cis)-6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo [1,5-a]pyridine]-1′,5′-dione,
(cis)-4-hydroxy-8′-methyl-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-4-(trifluoromethyl)-2′H-spiro[cyclohexane-1,3′-imidazo [1,5-a]pyridine]-1′,5′-dione,
(7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro [1]benzothieno[2,3-d]pyrimidine-7-carboxylic acid,
(7S)—N,N-dimethyl-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxamide,
6-{[(7S)-7-(azetidin-1-ylcarbonyl)-5,6,7,8-tetrahydro [1]benzothieno [2,3-d]pyrimidin-4-yl]amino}-8-methyl-2H-spiro[imidazo[1,5-a]pyridine-3,3′-thietane]-1,5-dione 1′,1′-dioxide,
1-({(7S)-4-[(8-methyl-1′,1′-dioxido-1,5-dioxo-1,5-dihydro-2H-spiro[imidazo[1,5-a]pyridine-3,3′-thietan]-6-yl)amino]-5,6,7,8-tetrahydro [1]benzothieno[2,3-d]pyrimidin-7-yl}carbonyl)azetidine-3-carbonitrile,
6-{[(7S)-7-{[3-(dimethylamino)azetidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide,
6-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide,
6-{[(7S)-7-{[(3R)-3,4-dimethylpiperazin-1-yl]carbonyl}-5,6,7,8-tetrahydro[1]benzothieno[2,3-d]pyrimidin-4-yl]amino}-8-methyl-2H-spiro[imidazo[1,5-a]pyridine-3,3′-thietane]-1,5-dione 1′,1′-dioxide,
8-methyl-6-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione 1′,1′-dioxide,
(7S)-4-[(8′-chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxylic acid,
(7S)-4-[(8′-chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidine-7-carboxamide,
(7S)-4-[(8′-chloro-1′,5′-dioxo-1′,5′-dihydro-2′H-spiro[cyclohexane-1,3′-imidazo[1,5-a]pyridin]-6′-yl)amino]-N,N-dimethyl-5,6,7,8-tetrahydro[1]benzothieno[2,3-d]pyrimidine-7-carboxamide,
6′-{[(7S)-7-(azetidin-1-ylcarbonyl)-5,6,7,8-tetrahydro[1]benzothieno[2,3-d]pyrimidin-4-yl]amino}-8′-chloro-2′H-spiro[cyclohexane-1,3′-imidazo[1,5-a]pyridine]-1′,5′-dione,
1-({(7S)-4-[(8′-chloro-1′,5′-dioxo-1′,5′-dihydro-2′H-spiro[cyclohexane-1,3′-imidazo[1,5-a]pyridin]-6′-yl)amino]-5,6,7,8-tetrahydro[1]benzothieno[2,3-d]pyrimidin-7-yl}carbonyl)azetidine-3-carbonitrile,
8′-chloro-6′-{[(7S)-7-{[(3S)-3-(dimethylamino)pyrrolidin-1-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione,
8′-chloro-6′-{[(7S)-7-{[(3R)-3-methylmorpholin-4-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione,
8′-chloro-6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-2′H-spiro[cyclohexane-1,3′-imidazo[1,5-a]pyridine]-1′,5′-dione,
8′-chloro-6′-({(7S)-7-[(1R,4R)-2-oxa-5-azabicyclo[2.2.1]hept-5-ylcarbonyl]-5,6,7,8-tetrahydro[1]benzothieno[2,3-d]pyrimidin-4-yl}amino)-2′H-spiro[cyclohexane-1,3′-imidazo[1,5-a]pyridine]-1′,5′-dione,
1-{[(7S)-4-{[(cis)-4-hydroxy-8′-methyl-1′,5′-dioxo-4-(trifluoromethyl)-1′,5′-dihydro-2′H-spiro[cyclohexane-1,3′-imidazo[1,5-a]pyridin]-6′-yl]amino}-5,6,7,8-tetrahydro[1]benzothieno[2,3-d]pyrimidin-7-yl]carbonyl}azetidine-3-carbonitrile,
(cis)-6′-{[(7S)-7-{[(3R)-3,4-dimethylpiperazin-1-yl]carbonyl}-5,6,7,8-tetrahydro[1]benzothieno[2,3-d]pyrimidin-4-yl]amino}-4-hydroxy-8′-methyl-4-(trifluoromethyl)-2′H-spiro[cyclohexane-1,3′-imidazo[1,5-a]pyridine]-1′,5′-dione,
(7S)-4-[(8′-chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxylic acid,
(7S)-4-[(8′-chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carboxamide,
(7S)-4-[(8′-chloro-1′,5′-dioxo-1′,5′-dihydro-2′H-spiro[cyclopentane-1,3′-imidazo[1,5-a]pyridin]-6′-yl)amino]-N,N-dimethyl-5,6,7,8-tetrahydro[1]benzothieno[2,3-d]pyrimidine-7-carboxamide,
6′-{[(7S)-7-(azetidine-1-carbonyl)-5,6,7,8-tetrahydro[1]benzothieno[2,3-d]pyrimidin-4-yl]amino}-8′-chloro-2′H-spiro[cyclopentane-1,3′-imidazo [1,5-a]pyridine]-1′,5′-dione,
1-{(7S)-4-[(8′-chloro-1′,5′-dioxo-1′,5′-dihydro-2′H-spiro[cyclopentane-1,3′-imidazo[1,5-a]pyridin]-6′-yl)amino]-5,6,7,8-tetrahydro[1]benzothieno[2,3-d]pyrimidine-7-carbonyl}azetidine-3-carbonitrile,
8′-chloro-6′-({(7S)-7-[(3S)-3-(dimethylamino)pyrrolidine-1-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione,
8′-chloro-6′-({(7S)-7-[(3R)-3-methylmorpholine-4-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione,
8′-chloro-6′-({(7S)-7-[(3R)-3,4-dimethylpiperazine-1-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione,
8′-chloro-6′-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2′H-spiro[cyclopentane-1,3′-imidazo[1,5-a]pyridine]-1′,5′-dione,
(7S)-4-[(8′-chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxylic acid,
(7S)-4-[(8′-chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carboxamide,
(7S)-4-[(8′-chloro-1′,5′-dioxo-1′,5′-dihydro-2′H-spiro[cyclobutane-1,3′-imidazo[1,5-a]pyridin]-6′-yl)amino]-N,N-dimethyl-5,6,7,8-tetrahydro[1]benzothieno[2,3-d]pyrimidine-7-carboxamide,
6′-{[(7S)-7-(azetidine-1-carbonyl)-5,6,7,8-tetrahydro[1]benzothieno[2,3-d]pyrimidin-4-yl]amino}-8′-chloro-2′H-spiro[cyclobutane-1,3′-imidazo[1,5-a]pyridine]-1′,5′-dione,
1-{(7S)-4-[(8′-chloro-1′,5′-dioxo-1′,5′-dihydro-2′H-spiro[cyclobutane-1,3′-imidazo[1,5-a]pyridin]-6′-yl)amino]-5,6,7,8-tetrahydro[1]benzothieno[2,3-d]pyrimidine-7-carbonyl}azetidine-3-carbonitrile,
8′-chloro-6′-({(7S)-7-[(3S)-3-(dimethylamino)pyrrolidine-1-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione,
8′-chloro-6′-({(7S)-7-[(3R)-3-methylmorpholine-4-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione,
8′-chloro-6′-({(7S)-7-[(3R)-3,4-dimethylpiperazine-1-carbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione,
(7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxylic acid,
(7S)-4-[(8-chloro-1,5-dioxo-1,5-dihydro-2H-spiro[imidazo[1,5-a]pyridine-3,3′-thietan]-6-yl)amino]-N,N-dimethyl-5,6,7,8-tetrahydro[1]benzothieno[2,3-d]pyrimidine-7-carboxamide,
6-{[(7S)-7-(azetidine-1-carbonyl)-5,6,7,8-tetrahydro[1]benzothieno[2,3-d]pyrimidin-4-yl]amino}-8-chloro-2H-spiro[imidazo [1,5-a]pyridine-3,3′-thietane]-1,5-dione,
1-{(7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carbonyl}azetidine-3-carbonitrile,
8-chloro-6-({(7S)-7-[(3S)-3-(dimethylamino)pyrrolidine-1-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione,
8-chloro-6-({(7S)-7-[(3R)-3-methylmorpholine-4-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione,
8-chloro-6-({(7S)-7-[(3R)-3,4-dimethylpiperazine-1-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione,
8-chloro-6-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2H-spiro[imidazo[1,5-a]pyridine-3,3′-thietane]-1,5-dione,
(7S)-4-[(8-chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxylic acid,
(7S)-4-[(8-chloro-3,3-dimethyl-1,5-dioxo-1,2,3,5-tetrahydroimidazo[1,5-a]pyridin-6-yl)amino]-5,6,7,8-tetrahydro[1]benzothieno[2,3-d]pyrimidine-7-carboxamide,
(7S)-4-[(8-chloro-3,3-dimethyl-1,5-dioxo-1,2,3,5-tetrahydroimidazo[1,5-a]pyridin-6-yl)amino]-N,N-dimethyl-5,6,7,8-tetrahydro[1]benzothieno[2,3-d]pyrimidine-7-carboxamide,
6-{[(7S)-7-(azetidine-1-carbonyl)-5,6,7,8-tetrahydro[1]benzothieno[2,3-d]pyrimidin-4-yl]amino}-8-chloro-3,3-dimethyl-2,3-dihydroimidazo [1,5-a]pyridine-1,5-dione,
1-{(7S)-4-[(8-chloro-3,3-dimethyl-1,5-dioxo-1,2,3,5-tetrahydroimidazo[1,5-a]pyridin-6-yl)amino]-5,6,7,8-tetrahydro [1]benzothieno[2,3-d]pyrimidine-7-carbonyl}azetidine-3-carbonitrile,
8-chloro-6-({(7S)-7-[(3S)-3-(dimethylamino)pyrrolidine-1-carbonyl]-5,6,7,8-tetrahydro[1]benzothieno[2,3-d]pyrimidin-4-yl}amino)-3,3-dimethyl-2,3-dihydroimidazo[1,5-a]pyridine-1,5-dione,
8-chloro-6-({(7S)-7-[(3R)-3,4-dimethylpiperazine-1-carbonyl]-5,6,7,8-tetrahydro[1]benzothieno[2,3-d]pyrimidin-4-yl}amino)-3,3-dimethyl-2,3-dihydroimidazo[1,5-a]pyridine-1,5-dione,
8-chloro-3,3-dimethyl-6-({(7S)-7-[(1R,4R)-2-oxa-5-azabicyclo[2.2.1]heptane-5-carbonyl]-5,6,7,8-tetrahydro[1]benzothieno[2,3-d]pyrimidin-4-yl}amino)-2,3-dihydroimidazo[1,5-a]pyridine-1,5-dione,
8-chloro-3,3-dimethyl-6-({(7S)-7-[(3R)-3-methylmorpholine-4-carbonyl]-5,6,7,8-tetrahydro[1]benzothieno[2,3-d]pyrimidin-4-yl}amino)-2,3-dihydroimidazo[1,5-a]pyridine-1,5-dione,
8′-chloro-6′-({(7S)-7-[(1R,4R)-2-oxa-5-azabicyclo[2.2.1]heptan-5-ylcarbonyl]-5,6,7,8-tetrahydro[1]benzothieno[2,3-d]pyrimidin-4-yl}amino)-2′H-spiro[cyclobutane-1,3′-imidazo[1,5-a]pyridine]-1′,5′-dione, and
(7S)-4-[(8-chloro-1,5-dioxo-1,5-dihydro-2H-spiro[imidazo[1,5-a]pyridine-3,3′-thietan]-6-yl)amino]-5,6,7,8-tetrahydro[1]benzothieno[2,3-d]pyrimidine-7-carboxamide;
or a tautomer, an N-oxide, a hydrate, a solvate, or a salt thereof, or a mixture of an of the foregoing.

10: A method of preparing the compound of formula (I) according to claim 1, comprising reacting an intermediate compound of formula (III): 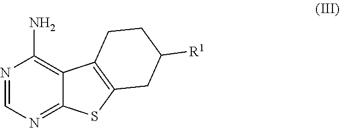
wherein
R¹ is as defined in claim 1 with an intermediate compound of formula (V): 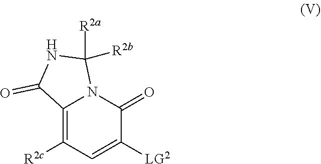
wherein
R^{2a}, R^{2b} and R^{2c} are as defined in claim 1, and LG² represents a leaving group LG.

11. (canceled)

12: A pharmaceutical composition comprising a compound of formula (I) according to claim 1, or a stereoisomer, a tautomer, an N-oxide, a hydrate, a solvate, or a salt thereof, or a mixture of any of the foregoing, and a pharmaceutically acceptable diluent or carrier.

13: A pharmaceutical combination comprising:
one or more first active ingredients selected from the compounds of formula (I) according to claim 1, or a stereoisomer, a tautomer, an N-oxide, a hydrate, a solvate, or a salt thereof, or a mixture of any of the foregoing, and
one or more second active ingredients selected from chemotherapeutic anti-cancer agents.

14-15. (canceled)

16: A method for prophylaxis or treatment of a disease, comprising administering to a patient in need thereof a pharmaceutically effective amount of a compound of formula (I) according to claim 1, or a stereoisomer, a tautomer, an N-oxide, a hydrate, a solvate, or a salt thereof, or a mixture of any of the foregoing, wherein said disease is a disease of uncontrolled cell growth, proliferation and/or survival, an inappropriate cellular immune response, or an inappropriate cellular inflammatory response, particularly in which the uncontrolled cell growth, proliferation and/or survival, inappropriate cellular immune response, or inappropriate cellular inflammatory response is mediated by the MKNK1 pathway, more particularly in which the disease of uncontrolled cell growth, proliferation and/or survival, inappropriate cellular immune response, or inappropriate cellular inflammatory response is a haematological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof, or pancreatitis, inflammatory and/or pain-associated diseases acute, chronic, inflammatory and neuropathic pain, preferably inflammatory pain, surgical pain, visceral pain, dental pain, premenstrual pain, endometriosis-associated pain, or other endometriosis-associated symptoms, wherein said symptoms are in particular endometriosis-associated dysmenorrhea, dyspareunia, dysuria, or dyschezia, pain associated with fibrotic diseases, central pain, pain due to burning mouth syndrome, pain due to burns, pain due to migraine, cluster headaches, pain due to nerve injury, pain due to neuritis, neuralgias, pain due to poisoning, pain due to ischemic injury, pain due to interstitial cystitis, cancer pain, pain due to viral, parasitic or bacterial infections, pain due to traumatic nerve-injury, pain due to post-traumatic injuries (including fractures and sport injuries), pain due to trigeminal neuralgia, pain associated with small fiber neuropathy, pain associated with diabetic neuropathy, chronic lower back pain, phantom limb pain, pelvic pain syndrome, chronic pelvic pain, neuroma pain, complex regional pain syndrome, pain associated with gastrointestinal distension, chronic arthritic pain and related neuralgias, and pain associated with cancer, pain associated with chemotherapy, HIV and HIV treatment-induced neuropathy; and pain associated with diseases or disorders selected from the group consisting of hyperalgesia, allodynia, irritable bowel syndrome.

17: A method for prophylaxis or treatment of a disease, comprising administering to a patient in need thereof a pharmaceutically effective amount of a compound of formula (I) according to claim 1, or a stereoisomer, a tautomer, an N-oxide, a hydrate, a solvate, or a salt thereof, or a mixture of the foregoing, wherein said disease is dysmenorrhea, dyspareunia, endometriosis, adenomyosis, endometriosis-associated pain, or other endometriosis-associated symptoms, wherein said symptoms are in particular endometriosis-associated proliferation, dysmenorrhea, dyspareunia, dysuria, or dyschezia.

18: A method for the treatment of inflammatory bowel disease (ulcerative colitis and Crohn's disease), hyperaemia, sepsis, metabolic disorders, e.g. obesity, insulin resistance, diabetes mellitus type 1 and 2, metabolic endocrine disorder; metabolic syndrome; atherosclerosis, reperfusion injury, inflammatory bone resorption, inflammatory liver diseases, pulmonary fibrosis, acute respiratory distress syndrome, and intestinal polyposis, inflammatory skin diseases like psoriasis, pemphigus vulgaris, inflammatory eye disorders like non-infectious uveitis, primary and secondary autoimmune uveitis, VKH syndrome, anterior uveitis, intermediate uveitis, posterior uveitis, panuveitis, Behcet's disease and neuromyelitis optica, fibrotic diseases like idiopathic pulmonary fibrosis, skin fibrosis, systemic sclerosis, autism disorders, liver diseases like nonalcoholic-, alcoholic- and toxic fatty liver disease, steatohepatitis, hepatic fibrosis; and cirrhosis; lung diseases like chronic obstructive pulmonary disease, asthma, pneumonia; neurodegenerative diseases like Parkinson's disease, Alzheimer's disease; stroke, postischemic brain injury, brain ischemia, post-traumatic brain injury, alopecia, acute coronary syndrome, myocardial infarction, autoimmune diseases like autoimmune encephalomyelitis, multiple sclerosis; arthritis (such as osteoarthritis and rheumatoid arthritis); psoriatic arthritis, ankylosing spondylitis), psoriasis, lupus erythematosus (e.g. systemic lupus erythematosus, cutaneous lupus and neonatal lupus erythematosus); inflammatory, autoimmune and fibrotic kidney diseases (e.g. glomerulonephritis, lupus nephritis, ANCA) interstitial cystitis, hypertrophy of the e.g. kidney, ischemia/reperfusion injury, allergic rhinitis, burn wound, osteoporosis viral and bacterial infections, chemotherapy-induced alopecia, cachexia induced for any reason, e.g. cancer, heart failure, etc., muscular atrophy, pancreatitis, schizophrenia, seizures, epilepsy, Fragile X syndrome, graft-versus-host disease, graft rejection, heart fibrosis, autoimmune myocardial disease, myocarditis, endocarditis, ischemia-reperfusion injury following e.g. myocardial infarction, hypertension, artherosclerosis, acute lung injury, ARDS, hypersensitivity pneumonitis, lung fibrosis, e.g. idiopathic pulmonary fibrosis, lymphocytic bronchiolitis e.g. after lung transplantation, dry eye, subfertility (e.g. associated with inflammatory conditions such as endometriosiso, or metabolic endocrine disorders), comprising administering to a patient in need thereof a pharmaceutically effective amount of a compound of formula (I) according to claim 1, or a stereoisomer, a tautomer, an N-oxide, a hydrate, a solvate, or a salt thereof, or a mixture of any of the foregoing.


SEQUENCE LISTING (5 sequences)

SEQ ID NO: 1 - protein, 14 aa, Unknown
  biotin-Ahx-IKKRKLTRRKSLKG
IKKRKLTRRKSLKG

SEQ ID NO: 2 - protein, 14 aa, Unknown
  biotin-Ahx-AEEEEYFELVAKKK
AEEEEYFELVAKKK

SEQ ID NO: 3 - protein, 14 aa, Unknown
  biotin-Ttds-YISPLKSPYKISEG
YISPLKSPYKISEG

SEQ ID NO: 4 - protein, 13 aa, Unknown
  biotin-KVEKIGEGTYGVV
KVEKIGEGTYGVV

SEQ ID NO: 5 - protein, 15 aa, Unknown
  Biotin-Ahx-GGEEEEYFELVKKKK
GGEEEEYFELVKKKK